SEQUENCE LISTING (255 sequences)

SEQ ID NO: 1 - DNA, 21 bp, Synthetic construct
  source 1..21: mol_type = other DNA
ttctgctgtgcagtgatgacg

SEQ ID NO: 2 - DNA, 21 bp, Synthetic construct
  source 1..21: mol_type = other DNA
tggaaaagctgatgaaggcct

SEQ ID NO: 3 - DNA, 21 bp, Synthetic construct
  source 1..21: mol_type = other DNA
tgctgcggaaaacactgaagg

SEQ ID NO: 4 - DNA, 21 bp, Synthetic construct
  source 1..21: mol_type = other DNA
cacggtgccgctcacacgggc

SEQ ID NO: 5 - DNA, 21 bp, Synthetic construct
  source 1..21: mol_type = other DNA
gtactgtgccagggcccgggc

SEQ ID NO: 6 - DNA, 21 bp, Synthetic construct
  source 1..21: mol_type = other DNA
ggacacaatggacaagaggtg

SEQ ID NO: 7 - DNA, 21 bp, Synthetic construct
  source 1..21: mol_type = other DNA
gaggaagctgagcattatcag

SEQ ID NO: 8 - DNA, 21 bp, Synthetic construct
  source 1..21: mol_type = other DNA
tgtcagcgcgtccaccagccc

SEQ ID NO: 9 - DNA, 21 bp, Synthetic construct
  source 1..21: mol_type = other DNA
atggatcccctccaagcactg

SEQ ID NO: 10 - DNA, 21 bp, Synthetic construct
  source 1..21: mol_type = other DNA
tttgcttggagatgaagaccc

SEQ ID NO: 11 - DNA, 21 bp, Synthetic construct
  source 1..21: mol_type = other DNA
cctcagggtgagcagcacgcc

SEQ ID NO: 12 - DNA, 21 bp, Synthetic construct
  source 1..21: mol_type = other DNA
ggcacagtggtaaatgatgga

SEQ ID NO: 13 - DNA, 21 bp, Synthetic construct
  source 1..21: mol_type = other DNA
tgggaaccaagctgacgagag

SEQ ID NO: 14 - DNA, 21 bp, Synthetic construct
  source 1..21: mol_type = other DNA
ctcgaacaagcaagagacccg

SEQ ID NO: 15 - DNA, 21 bp, Synthetic construct
  source 1..21: mol_type = other DNA
cattgactctcatgaagccct

SEQ ID NO: 16 - DNA, 21 bp, Synthetic construct
  source 1..21: mol_type = other DNA
tggaagaactcaacagaatcc

SEQ ID NO: 17 - DNA, 21 bp, Synthetic construct
  source 1..21: mol_type = other DNA
ggatgaacaggccgctggcag

SEQ ID NO: 18 - DNA, 21 bp, Synthetic construct
  source 1..21: mol_type = other DNA
ttgatccatgagcagatcccg

SEQ ID NO: 19 - DNA, 21 bp, Synthetic construct
  source 1..21: mol_type = other DNA
acaagtaaatcctcatcactg

SEQ ID NO: 20 - DNA, 21 bp, Synthetic construct
  source 1..21: mol_type = other DNA
gggtgatgctgtctggaagtg

SEQ ID NO: 21 - DNA, 21 bp, Synthetic construct
  source 1..21: mol_type = other DNA
atacggagagaaggagagctc

SEQ ID NO: 22 - DNA, 21 bp, Synthetic construct
  source 1..21: mol_type = other DNA
cagcattccctgagatccccg

SEQ ID NO: 23 - DNA, 21 bp, Synthetic construct
  source 1..21: mol_type = other DNA
gctggtcaagctgagtgtgga

SEQ ID NO: 24 - DNA, 21 bp, Synthetic construct
  source 1..21: mol_type = other DNA
gctccatgcttaagcctaggg

SEQ ID NO: 25 - DNA, 21 bp, Synthetic construct
  source 1..21: mol_type = other DNA
ggcctgcagattggacagccc

SEQ ID NO: 26 - DNA, 21 bp, Synthetic construct
  source 1..21: mol_type = other DNA
cactatggaaaataacctttc

SEQ ID NO: 27 - DNA, 21 bp, Synthetic construct
  source 1..21: mol_type = other DNA
tcccatcattcaggtccatgg

SEQ ID NO: 28 - DNA, 21 bp, Synthetic construct
  source 1..21: mol_type = other DNA
gtcctgcagcagtgccacaag

SEQ ID NO: 29 - DNA, 21 bp, Synthetic construct
  source 1..21: mol_type = other DNA
gcacgccaagaatcagcagag

SEQ ID NO: 30 - DNA, 21 bp, Synthetic construct
  source 1..21: mol_type = other DNA
gtgagatggtggcagaaatgg

SEQ ID NO: 31 - DNA, 21 bp, Synthetic construct
  source 1..21: mol_type = other DNA
agagacgaaacctgtccagca

SEQ ID NO: 32 - DNA, 21 bp, Synthetic construct
  source 1..21: mol_type = other DNA
acagtagatgagggagcaggc

SEQ ID NO: 33 - DNA, 21 bp, Synthetic construct
  source 1..21: mol_type = other DNA
gaaaggctttccttgtgaagc

SEQ ID NO: 34 - DNA, 21 bp, Synthetic construct
  source 1..21: mol_type = other DNA
gcatcacacccctgagggagg

SEQ ID NO: 35 - DNA, 21 bp, Synthetic construct
  source 1..21: mol_type = other DNA
cccaatgcccccgactgcggt

SEQ ID NO: 36 - DNA, 21 bp, Synthetic construct
  source 1..21: mol_type = other DNA
gacaccagagcagcaccaggg

SEQ ID NO: 37 - DNA, 21 bp, Synthetic construct
  source 1..21: mol_type = other DNA
ccccagatgtctggagaagag

SEQ ID NO: 38 - DNA, 21 bp, Synthetic construct
  source 1..21: mol_type = other DNA
tgcctccactgagtgcctcag

SEQ ID NO: 39 - DNA, 21 bp, Synthetic construct
  source 1..21: mol_type = other DNA
cttttcctggaggaactccac

SEQ ID NO: 40 - DNA, 21 bp, Synthetic construct
  source 1..21: mol_type = other DNA
gaaggcattcatatcttcagc

SEQ ID NO: 41 - DNA, 21 bp, Synthetic construct
  source 1..21: mol_type = other DNA
cacggtgccgctcacacgggc

SEQ ID NO: 42 - DNA, 21 bp, Synthetic construct
  source 1..21: mol_type = other DNA
ctcgaacaagcaagagacccg

SEQ ID NO: 43 - DNA, 21 bp, Synthetic construct
  source 1..21: mol_type = other DNA
tgctgcggaaaacactgaagg

SEQ ID NO: 44 - DNA, 21 bp, Synthetic construct
  source 1..21: mol_type = other DNA
tgctgcggaaaacactgaagg

SEQ ID NO: 45 - DNA, 21 bp, Synthetic construct
  source 1..21: mol_type = other DNA
cagcattccctgagatccccg

SEQ ID NO: 46 - DNA, 21 bp, Synthetic construct
  source 1..21: mol_type = other DNA
cctcagggtgagcagcacgcc

SEQ ID NO: 47 - DNA, 21 bp, Synthetic construct
  source 1..21: mol_type = other DNA
gaggaagctgagcattatcag

SEQ ID NO: 48 - DNA, 21 bp, Synthetic construct
  source 1..21: mol_type = other DNA
actccaaatgcaaactgggaa

SEQ ID NO: 49 - DNA, 21 bp, Synthetic construct
  source 1..21: mol_type = other DNA
tttgcttggagatgaagaccc

SEQ ID NO: 50 - DNA, 21 bp, Synthetic construct
  source 1..21: mol_type = other DNA
ttgatccatgagcagatcccg

SEQ ID NO: 51 - DNA, 21 bp, Synthetic construct
  source 1..21: mol_type = other DNA
gaaaggctttccttgtgaagc

SEQ ID NO: 52 - DNA, 21 bp, Synthetic construct
  source 1..21: mol_type = other DNA
ttctgctgtgcagtgatgacg

SEQ ID NO: 53 - DNA, 21 bp, Synthetic construct
  source 1..21: mol_type = other DNA
acaagtaaatcctcatcactg

SEQ ID NO: 54 - DNA, 21 bp, Synthetic construct
  source 1..21: mol_type = other DNA
ggatgaacaggccgctggcag

SEQ ID NO: 55 - DNA, 21 bp, Synthetic construct
  source 1..21: mol_type = other DNA
agagacgaaacctgtccagca

SEQ ID NO: 56 - DNA, 21 bp, Synthetic construct
  source 1..21: mol_type = other DNA
cagatgtcaggatggtggctg

SEQ ID NO: 57 - DNA, 21 bp, Synthetic construct
  source 1..21: mol_type = other DNA
tgtcagcgcgtccaccagccc

SEQ ID NO: 58 - DNA, 21 bp, Synthetic construct
  source 1..21: mol_type = other DNA
gctccatgcttaagcctaggg

SEQ ID NO: 59 - DNA, 21 bp, Synthetic construct
  source 1..21: mol_type = other DNA
tgggaaccaagctgacgagag

SEQ ID NO: 60 - DNA, 21 bp, Synthetic construct
  source 1..21: mol_type = other DNA
tggaagaactcaacagaatcc

SEQ ID NO: 61 - DNA, 21 bp, Synthetic construct
  source 1..21: mol_type = other DNA
tgtgacgctgacagaactgcg

SEQ ID NO: 62 - DNA, 21 bp, Synthetic construct
  source 1..21: mol_type = other DNA
cattgactctcatgaagccct

SEQ ID NO: 63 - DNA, 21 bp, Synthetic construct
  source 1..21: mol_type = other DNA
gggtgatgctgtctggaagtg

SEQ ID NO: 64 - DNA, 21 bp, Synthetic construct
  source 1..21: mol_type = other DNA
ggatggtcacccaaaccggga

SEQ ID NO: 65 - DNA, 21 bp, Synthetic construct
  source 1..21: mol_type = other DNA
gcagtcgtccatggtccggga

SEQ ID NO: 66 - DNA, 21 bp, Synthetic construct
  source 1..21: mol_type = other DNA
gctggtcaagctgagtgtgga

SEQ ID NO: 67 - DNA, 21 bp, Synthetic construct
  source 1..21: mol_type = other DNA
atggatcccctccaagcactg

SEQ ID NO: 68 - DNA, 21 bp, Synthetic construct
  source 1..21: mol_type = other DNA
ggcacagtggtaaatgatgga

SEQ ID NO: 69 - DNA, 21 bp, Synthetic construct
  source 1..21: mol_type = other DNA
gaatctgacctggtccaacac

SEQ ID NO: 70 - DNA, 21 bp, Synthetic construct
  source 1..21: mol_type = other DNA
gtcctgcagcagtgccacaag

SEQ ID NO: 71 - DNA, 21 bp, Synthetic construct
  source 1..21: mol_type = other DNA
tgcacggcgtcctctatgtgc

SEQ ID NO: 72 - DNA, 21 bp, Synthetic construct
  source 1..21: mol_type = other DNA
attccggccctacgtccagag

SEQ ID NO: 73 - DNA, 21 bp, Synthetic construct
  source 1..21: mol_type = other DNA
gtactgtgccagggcccgggc

SEQ ID NO: 74 - DNA, 21 bp, Synthetic construct
  source 1..21: mol_type = other DNA
ccccagatgtctggagaagag

SEQ ID NO: 75 - DNA, 21 bp, Synthetic construct
  source 1..21: mol_type = other DNA
gtgagatggtggcagaaatgg

SEQ ID NO: 76 - DNA, 21 bp, Synthetic construct
  source 1..21: mol_type = other DNA
catgcgagccagcacacggaa

SEQ ID NO: 77 - DNA, 21 bp, Synthetic construct
  source 1..21: mol_type = other DNA
gaaggcattcatatcttcagc

SEQ ID NO: 78 - DNA, 21 bp, Synthetic construct
  source 1..21: mol_type = other DNA
acagtagatgagggagcaggc

SEQ ID NO: 79 - DNA, 21 bp, Synthetic construct
  source 1..21: mol_type = other DNA
tctggagcgtggtctcctcca

SEQ ID NO: 80 - DNA, 21 bp, Synthetic construct
  source 1..21: mol_type = other DNA
agtgaaatttctggtggccag

SEQ ID NO: 81 - DNA, 20 bp, Synthetic construct
  source 1..20: mol_type = other DNA
caagaccacaatgttgtgac

SEQ ID NO: 82 - DNA, 20 bp, Synthetic construct
  source 1..20: mol_type = other DNA
cttcccaatgtcctgcagtg

SEQ ID NO: 83 - DNA, 20 bp, Synthetic construct
  source 1..20: mol_type = other DNA
agaatcagtccaggagacct

SEQ ID NO: 84 - DNA, 20 bp, Synthetic construct
  source 1..20: mol_type = other DNA
ggaaatcagaaccctcaaaa

SEQ ID NO: 85 - DNA, 20 bp, Synthetic construct
  source 1..20: mol_type = other DNA
ccagaggatagttctcaatg

SEQ ID NO: 86 - DNA, 20 bp, Synthetic construct
  source 1..20: mol_type = other DNA
caggatcctggccaccactc

SEQ ID NO: 87 - DNA, 20 bp, Synthetic construct
  source 1..20: mol_type = other DNA
gtagtagccggagacagaga

SEQ ID NO: 88 - DNA, 20 bp, Synthetic construct
  source 1..20: mol_type = other DNA
ttaagtgctcggagtcatgg

SEQ ID NO: 89 - DNA, 20 bp, Synthetic construct
  source 1..20: mol_type = other DNA
ttccatgatcaccacccacc

SEQ ID NO: 90 - DNA, 20 bp, Synthetic construct
  source 1..20: mol_type = other DNA
attgtgtccttcttgagtgc

SEQ ID NO: 91 - DNA, 20 bp, Synthetic construct
  source 1..20: mol_type = other DNA
gcacagtggtaaatgatgga

SEQ ID NO: 92 - DNA, 20 bp, Synthetic construct
  source 1..20: mol_type = other DNA
gatgagggtcccacagagaa

SEQ ID NO: 93 - DNA, 20 bp, Synthetic construct
  source 1..20: mol_type = other DNA
tcatcgatgtgctgactctg

SEQ ID NO: 94 - DNA, 20 bp, Synthetic construct
  source 1..20: mol_type = other DNA
gtttgctgaagaaagattcc

SEQ ID NO: 95 - DNA, 20 bp, Synthetic construct
  source 1..20: mol_type = other DNA
taatgtatttaacactccaa

SEQ ID NO: 96 - DNA, 20 bp, Synthetic construct
  source 1..20: mol_type = other DNA
actttctattgccaggaact

SEQ ID NO: 97 - DNA, 20 bp, Synthetic construct
  source 1..20: mol_type = other DNA
caatctgcaggcccaaatac

SEQ ID NO: 98 - DNA, 20 bp, Synthetic construct
  source 1..20: mol_type = other DNA
catcgatcatggagacccac

SEQ ID NO: 99 - DNA, 20 bp, Synthetic construct
  source 1..20: mol_type = other DNA
atgtgtgcagttacagaagc

SEQ ID NO: 100 - DNA, 20 bp, Synthetic construct
  source 1..20: mol_type = other DNA
agacgacttcttcccacccc

SEQ ID NO: 101 - DNA, 20 bp, Synthetic construct
  source 1..20: mol_type = other DNA
gagcattatcagagggattg

SEQ ID NO: 102 - DNA, 20 bp, Synthetic construct
  source 1..20: mol_type = other DNA
cagctcagcaaacctccaca

SEQ ID NO: 103 - DNA, 20 bp, Synthetic construct
  source 1..20: mol_type = other DNA
ggtaccgacaaccagtattt

SEQ ID NO: 104 - DNA, 20 bp, Synthetic construct
  source 1..20: mol_type = other DNA
gggctccggtcacaacattg

SEQ ID NO: 105 - DNA, 20 bp, Synthetic construct
  source 1..20: mol_type = other DNA
ggagattaaatacggagaga

SEQ ID NO: 106 - DNA, 20 bp, Synthetic construct
  source 1..20: mol_type = other DNA
atcactgcggaacagcctag

SEQ ID NO: 107 - DNA, 20 bp, Synthetic construct
  source 1..20: mol_type = other DNA
tctcttgccacggccactac

SEQ ID NO: 108 - DNA, 20 bp, Synthetic construct
  source 1..20: mol_type = other DNA
gaggaacagttcctattggc

SEQ ID NO: 109 - DNA, 20 bp, Synthetic construct
  source 1..20: mol_type = other DNA
gtcctgtactggaggctcgt

SEQ ID NO: 110 - DNA, 20 bp, Synthetic construct
  source 1..20: mol_type = other DNA
ataatgtatttaacactcca

SEQ ID NO: 111 - DNA, 20 bp, Synthetic construct
  source 1..20: mol_type = other DNA
atgcgagccagcacacggaa

SEQ ID NO: 112 - DNA, 20 bp, Synthetic construct
  source 1..20: mol_type = other DNA
gacatcatgaacaaagtcat

SEQ ID NO: 113 - DNA, 20 bp, Synthetic construct
  source 1..20: mol_type = other DNA
gttacagctcgagctctata

SEQ ID NO: 114 - DNA, 20 bp, Synthetic construct
  source 1..20: mol_type = other DNA
gctgcacggtgccgctcaca

SEQ ID NO: 115 - DNA, 20 bp, Synthetic construct
  source 1..20: mol_type = other DNA
aaatgttaatcaccttcagc

SEQ ID NO: 116 - DNA, 20 bp, Synthetic construct
  source 1..20: mol_type = other DNA
tcaggacatcatgcagtttg

SEQ ID NO: 117 - DNA, 20 bp, Synthetic construct
  source 1..20: mol_type = other DNA
gatcaccacccacccggccc

SEQ ID NO: 118 - DNA, 20 bp, Synthetic construct
  source 1..20: mol_type = other DNA
tagagaaatagtacgaagag

SEQ ID NO: 119 - DNA, 20 bp, Synthetic construct
  source 1..20: mol_type = other DNA
agtttgctgaagaaagattc

SEQ ID NO: 120 - DNA, 20 bp, Synthetic construct
  source 1..20: mol_type = other DNA
ggaagaagtcgtctagaaac

SEQ ID NO: 121-240 - skipped (120 entries)

SEQ ID NO: 241 - DNA, 145 bp, Synthetic construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 242 - DNA, 82 bp, Synthetic construct
  source 1..82: mol_type = other DNA
gttttagtactctggaaacagaatctactaaaacaaggcaaaatgccgtgtttatctcgtcaacttgttggcgagatttttt

SEQ ID NO: 243 - DNA, 82 bp, Synthetic construct; identical to SEQ ID NO: 242
  source 1..82: mol_type = other DNA

SEQ ID NO: 244 - DNA, 82 bp, Synthetic construct
  source 1..82: mol_type = other DNA
gttttagagctagaaatagcaagttaaaataaggctagtccgttatcaacttgaaaaagtggcaccgagtcggtgctttttt

SEQ ID NO: 245 - DNA, 249 bp, Synthetic construct
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 246 - DNA, 772 bp, Synthetic construct
  source 1..772: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 247 - DNA, 403 bp, Synthetic construct
  source 1..403: mol_type = other DNA
tcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcggccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctag

SEQ ID NO: 248 - DNA, 448 bp, Synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 249 - DNA, 229 bp, Synthetic construct
  source 1..229: mol_type = other DNA
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 250 - DNA, 184 bp, Synthetic construct
  source 1..184: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgata

SEQ ID NO: 251 - DNA, 3255 bp, Synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 252 - DNA, 3255 bp, Synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagaaagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaagaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggcccccccacatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 253 - DNA, 4197 bp, Synthetic construct
  source 1..4197: mol_type = other DNA
atgccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaag

SEQ ID NO: 254 - DNA, 60 bp, Synthetic construct
  source 1..60: mol_type = other DNA
tagcaataaaggatcgtttattttcattggaagcgtgtgttggttttttgatcaggcgcg

SEQ ID NO: 255 - DNA, 232 bp, Synthetic construct
  source 1..232: mol_type = other DNA
ctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

(19) United States
(12) Patent Application Publication
(10) Pub. No.: US 2025/0360228 A1
(43) Pub. Date: Nov. 27, 2025

(54) TREATMENT OF GENETIC NEUROLOGICAL CONDITIONS WITH GENOMIC EDITING

(71) Applicant: Sarepta Therapeutics, Inc., Cambridge, MA (US)

(72) Inventors: Ami M. KABADI, Cambridge, MA (US); Xinzhu WANG, Cambridge, MA (US)

(73) Assignee: Sarepta Therapeutics, Inc., Cambridge, MA (US)

(21) Appl. No.: 19/187,380

(22) Filed: Apr. 23, 2025

Related U.S. Application Data
(60) Provisional application No. 63/638,225, filed on Apr. 24, 2024.

(51) Int. Cl. A61K 48/00; C12N 9/22; C12N 15/11; C12N 15/86

(52) U.S. Cl. CPC A61K 48/0058; C12N 9/226; C12N 15/11; C12N 15/86; C12N 2310/20; C12N 2750/14143

(57) ABSTRACT

The present disclosure provides methods and compositions concerning the CRISPR/Cas9 systems and associated guide RNAs, which target and excise portions of particular exons of a huntingtin gene, thereby abrogating huntingtin protein expression. The disclosure further provides methods and compositions for treating Huntington's Disease.

### CROSS REFERENCE TO RELATED APPLICATIONS

This application claims the benefit of U.S. Provisional Appl. No. 63/638,225, filed Apr. 24, 2024, the contents of which are hereby incorporated by reference in their entireties.

### REFERENCE TO SEQUENCE LISTING SUBMITTED ELECTRONICALLY

The content of the electronically submitted sequence listing in text format (4140_1250001_Sequencelisting_ST26.xml; Size: 157,302 bytes; Date of Creation: Apr. 22, 2025) filed with the application is incorporated by reference in its entirety.

### FIELD OF DISCLOSURE

The present disclosure relates to the field of compositions and methods for the treatment of a genetic neurological condition (e.g., Huntington's Disease) through genome engineering and genomic alteration of the gene responsible for the expression of the huntingtin polypeptide using Clustered Regularly Interspaced Short Palindromic Repeats (CRISPR)/CRISPR-associated (Cas) 9-based systems and viral delivery systems. The present disclosure also relates to the field of genome engineering and genomic alteration by driving expression of CRISPR/Cas9 systems in neurons and related tissues, such as glial cells and astrocytes, thereby treating a genetic neurological condition (e.g., Huntington's Disease).

### BACKGROUND

Huntingtin is a polypeptide product of the human HTT gene. Located on chromosome 4 at locus 4p16.3, the human HTT gene contains 67 exons and spans approximately 180 kilobases (Ambrose, C. M., et al. (1994). Somatic Cell and Molecular Genetics, 20, 27-38). While the exact cellular functions of the huntingtin polypeptide are not known, the protein has been shown to impact at least the early development of neurons and cellular trafficking (White, J. K., et al. (1997). Nature Genetics, 17 (4), 404-410; Colin, E., et al. (2008). The EMBO Journal, 27 (15), 2124-2134). Mutations in the HTT gene, particularly expansion of a poly-Q region in exon 1 (i.e., repeats of the CAG trinucleotide), causes the resultant polypeptide product to induce the neurodegeneration associated with Huntington's Disease, and while the exact pathological mechanism of action remains unclear, it is noted that mitochondria are particularly affected by the disease (Kim, J., et al. (2010). Human Molecular Genetics, 19 (20), 3919-3935; Franco-Iborra, S., et al. (2021). Autophagy, 17 (3), 672-689.) and that this poly-Q stretch enables the huntingtin protein to form aggregates in neurons of the striatum (e.g., medium spiny neurons) and/or the cortex (Jarosińska, O. D., & Rüdiger, S. G. (2021). Frontiers in Molecular Biosciences, 8, 1068).

Huntington's Disease (HD) affects as many as 5-10 people per 100,000 worldwide and is typically diagnosed as a result of neurological symptoms (Reiner, A., et al. (2011). International Review of Neurobiology, 98, 325-372). Onset typically occurs in individuals aged 30 to 50 years and is characterized by progressively worsening motor function (e.g., involuntary movements), cognition and psychiatric symptoms (e.g., new or worsening anxiety and/or depression) (Bates, G. P., et al. (2015). Nature Reviews: Disease Primers, 1 (1), 1-21). The disease is invariably fatal within 10 to 25 years from onset, and no treatment currently exists to modify its course.

In view of the inevitable lethality and the severe impacts on quality of life associated with HD and the lack of current treatment options, it is imperative that permanent, course-altering therapies be developed to aid this afflicted population. Some proposed therapies, such as using RNA interference to halt huntingtin expression (Wild, E. J., & Tabrizi, S. J. (2017). The Lancet Neurology, 16 (10), 837-847), show promise but would require regular re-dosing to ensure that protein expression remains depressed. To this end, a therapeutic approach that permanently depresses activity of the HTT gene would represent a therapy capable of aiding the HD patient population with a single therapeutic intervention without the need for re-dosing.

### BRIEF SUMMARY OF THE DISCLOSURE

The present disclosure concerns methods and compositions for the treatment of one or more genetic neurological conditions. In preferred embodiments, the genetic neurological condition is Huntington's Disease (HD).

In one aspect, the composition comprises a CRISPR based gene editing system comprising one or more polynucleotides, wherein the one or more polynucleotides encode a composition that comprises a Cas protein or a fusion protein comprising the Cas protein or its component, and a gRNA, wherein the gRNA comprises a sequence selected from SEQ ID NOs: 1-120. In some embodiments, the gRNA targets an exon of a HTT gene selected from any one of exons 1-63, exon 65, and exon 66. In some embodiments, the Cas protein is a type II Cas enzyme or a type V Cas enzyme. In some embodiments, the Cas protein is a Cas9 protein.

In some embodiments, the Cas9 protein is a SaCas9 protein, and the gRNA comprises a sequence selected from any one of SEQ ID NOs: 1-40. In a preferred embodiment, the gRNA comprises SEQ ID NO: 1. In some embodiments, the SaCa9 protein recognizes a protospacer-adjacent motif (PAM) comprising SEQ ID NO: 121. In some embodiments, the gRNA targets exon 3 of a HTT gene. In a preferred embodiment, the gRNA comprises SEQ ID NO: 2. In some embodiments, the SaCa9 protein recognizes a PAM comprising SEQ ID NO: 122. In some embodiments, the gRNA targets exon 1 of a HTT gene.

In some embodiments, the Cas9 protein is a KKH-SaCas9 protein, and the gRNA comprises a sequence selected from any one of SEQ ID NOs: 41-80. In a preferred embodiment, the gRNA comprises SEQ ID NO: 41. In some embodiments, the KKH-SaCa9 protein recognizes a PAM comprising SEQ ID NO: 161. In some embodiments, the gRNA targets exon 48 of a HTT gene. In a preferred embodiment, the gRNA comprises SEQ ID NO: 42. In some embodiments, the KKH-SaCa9 protein recognizes a PAM comprising SEQ ID NO: 162.

In some embodiments, the Cas9 protein is a SpCas9 protein; and wherein the gRNA comprises a sequence selected from any one of SEQ ID NOs: 81-120. In a preferred embodiment, the gRNA comprises SEQ ID NO: 81. In some embodiments, the SpCas9 protein recognizes a PAM comprising SEQ ID NO: 201. In some embodiments, the gRNA targets exon 9 of a HTT gene. In a preferred embodiment, the gRNA comprises SEQ ID NO: 82. In some embodiments, the SpCas9 protein recognizes a PAM comprising SEQ ID NO: 202. In some embodiments, the gRNA targets exon 29 of a HTT gene. In a preferred embodiment, the gRNA comprises SEQ ID NO: 83. In some embodiments, the SpCas9 protein recognizes a PAM comprising SEQ ID NO: 203. In some embodiments, the gRNA targets exon 6 of a HTT gene. In a preferred embodiment, the gRNA comprises SEQ ID NO: 84. In some embodiments, the SpCas9 protein recognizes a PAM comprising SEQ ID NO: 204. In some embodiments, the gRNA targets exon 39 of a HTT gene. In a preferred embodiment, the gRNA comprises SEQ ID NO: 85. In some embodiments, the SpCas9 protein recognizes a PAM comprising SEQ ID NO: 205. In some embodiments, the gRNA targets exon 62 of a HTT gene. In a preferred embodiment, the gRNA comprises SEQ ID NO: 86. In some embodiments, the SpCas9 protein recognizes a PAM comprising SEQ ID NO: 206. In some embodiments, the gRNA targets exon 65 of a HTT gene. In a preferred embodiment, the gRNA comprises SEQ ID NO: 87. In some embodiments, the SpCas9 protein recognizes a PAM comprising SEQ ID NO: 207. In some embodiments, the gRNA targets exon 56 of a HTT gene. In a preferred embodiment, the gRNA comprises SEQ ID NO: 88. In some embodiments, the SpCas9 protein recognizes a PAM comprising SEQ ID NO: 208. In some embodiments, the gRNA targets exon 43 of a HTT gene. In a preferred embodiment, the gRNA comprises SEQ ID NO: 89. In some embodiments, the SpCas9 protein recognizes a PAM comprising SEQ ID NO: 209. In some embodiments, the gRNA targets exon 41 of a HTT gene. In a preferred embodiment, the gRNA comprises SEQ ID NO: 90. In some embodiments, the SpCas9 protein recognizes a PAM comprising SEQ ID NO: 210. In some embodiments, the gRNA targets exon 7 of a HTT gene. In a preferred embodiment, the gRNA comprises SEQ ID NO: 91. In some embodiments, the SpCas9 protein recognizes a PAM comprising SEQ ID NO: 211. In some embodiments, the gRNA targets exon 63 of a HTT gene. In a preferred embodiment, the gRNA comprises SEQ ID NO: 92. In some embodiments, the SpCas9 protein recognizes a PAM comprising SEQ ID NO: 212. In some embodiments, the gRNA targets exon 17 of a HTT gene.

In one aspect, the CRISPR-based gene editing system introduces a double stranded break at a target nucleic acid sequence. In some embodiments, the expression of the Cas9 protein is driven by a constitutive promoter or a neuron-specific promoter. In some embodiments, the constitutive promoter comprises a CBh promoter, an EFS promoter, an SCP1 promoter, an SCP3 promoter or a JeT promoter. In some embodiments, the neuron-specific promoter comprises a E/hSyn promoter or a E/hMeCP2 promoter. In some embodiments, the Cas protein and the gRNA are encoded by a single vector. In some embodiments, the Cas protein is encoded by a first vector and the gRNA is encoded by a second vector. In some embodiments, the vector is a viral vector. In some embodiments, the viral vector is an adeno-associated virus (AAV) vector. In some embodiments, the AAV vector is selected from AAV1, AAV2, AAV3, AAV4, AAV5, AAV6, AAV7, AAV8, AAV9, AAV-10, AAV-11, AAV-12, AAV-13, AAVrh.74, or a recombinant variant thereof. In some embodiments, the vector comprises a ubiquitous promoter or a tissue-specific promoter operably linked to the polynucleotide sequence encoding the Cas protein and/or the gRNA. In some embodiments, the tissue-specific promoter is a neuron-specific promoter.

In one aspect, the compositions include a CRISPR-based gene editing system or vector comprising a CRISPR-based gene editing system as a component of a cell. In some embodiments, the cell is a eukaryotic cell. In some embodiments, the cell is a prokaryotic cell. In some embodiments, the cell is a yeast cell. In some embodiments, the cell is an insect cell. In some embodiments, the cell is a mammalian cell. In some embodiments, the cell is a bacterial cell. In some embodiments, the cell is a stem cell. In some embodiments, the cell is a neuron. In some embodiments, the cell is a glial cell. In some embodiments, the cell is an astrocyte. In some embodiments, the cell is a HeLa cell. In some embodiments, the cell is a 293 cell. In some embodiments, the cell is a PerC.6 In some embodiments, the cell is a Sf9 cell. In another aspect, the compositions include a CRISPR-based gene editing system or vector comprising a CRISPR-based gene editing system as a component of a kit.

In one aspect, the present disclosure includes methods associated with genomic engineering to treat a genetic disorder. In some embodiments, the method comprises modifying a mutant huntingtin gene in a cell, the method comprising administering to the cell a CRISPR-based gene editing system or a vector comprising a CRISPR-based gene editing system. In some embodiments, the method comprises modifying a mutant huntingtin gene in a subject, the method comprising administering to the subject a CRISPR-based gene editing system, a vector comprising a CRISPR-based gene editing system, or a cell comprising a CRISPR-based gene editing system. In some embodiments, the method comprises treating a subject having a mutant huntingtin gene, the method comprising administering to the subject a CRISPR-based gene editing system, a vector comprising a CRISPR-based gene editing system, or a cell comprising a CRISPR-based gene editing system. In some embodiments, the method comprises treating a disease in a patient in need thereof, the method comprising administering to the subject a CRISPR-based gene editing system, a vector comprising a CRISPR-based gene editing system, or a cell comprising a CRISPR-based gene editing system. In some embodiments, the disease is Huntington's Disease. In some embodiments, the method further comprises intravenous administration of a CRISPR-based gene editing system, a vector comprising a CRISPR-based gene editing system, or a cell comprising a CRISPR-based gene editing system. In some embodiments, the method further comprises intracranial administration of a CRISPR-based gene editing system, a vector comprising a CRISPR-based gene editing system, or a cell comprising a CRISPR-based gene editing system. In some embodiments, the method further comprises a combination of intravenous and intracranial administration of a CRISPR-based gene editing system, a vector comprising a CRISPR-based gene editing system, or a cell comprising a CRISPR-based gene editing system. In preferred embodiments, the detectable amount of huntingtin protein is reduced by at least about 50%, as compared to an unmodified control. In preferred embodiments, the detectable amount of huntingtin protein is reduced by at least about 55%, as compared to an unmodified control. In preferred embodiments, the detectable amount of huntingtin protein is reduced by at least about 60%, as compared to an unmodified control. In preferred embodiments, the detectable amount of huntingtin protein is reduced by at least about 70%, as compared to an unmodified control. In preferred embodiments, the detectable amount of huntingtin protein is reduced by at least about 75%, as compared to an unmodified control.

### BRIEF DESCRIPTION OF DRAWINGS

FIG. 1 illustrates the workflow for the huntingtin knockout screening approach to identify gRNAs capable of ablating HTT gene expression. In silico design of 1,999 unique gRNAs was followed by generation of a lentivirus library, in which 1,793 of these were packaged into lentivirus vectors for screening in eHAP1 cells before FACS, PCR amplification and next-generation sequencing analyses.

FIGS. 2A, 2B 2C, and 2D collectively illustrate validation of FACS gating protocols used in screening the lentivirus library, including (A) positive and (B) negative controls on wildtype huntingtin-expressing cells, (C) antibody control with a clonally expanded HTT knockout cell line, and (D) exemplary data illustrating the sorted cell populations based upon relative huntingtin expression.

FIG. 3 illustrates selection criteria for narrowing to top 40 gRNAs for further validation experiments.

FIGS. 4A, 4B, and 4C collectively illustrate relative abundance of gRNAs in the Low, High, and General populations before and after applying the aforementioned selection criteria to lentivirus-transduced cells stably expressing (A) SaCas9, (B) KKH-SaCas9, or (C) SpCas9 on the basis of levels of detected huntingtin protein via FACS.

FIGS. 5A, 5B, 5C, 5D, 5E, 5F, 5G, 5H, 5I, and 5J collectively illustrate relative levels of huntingtin protein detected following treatment with the selected gRNAs and the appropriate Cas9 nuclease (as noted in Tables 1-3).

FIGS. 6A, 6B, 6C, 6D, and 6E collectively illustrate validation of the top-performing SaCas9 gRNAs by (A) confirmatory western blot, (B) quantification of replicate blots, Sanger sequencing of cells treated with (C) SEQ ID NO: 1 and (D) SEQ ID NO: 2, and (E) quantification of knockouts generated from sequencing cells treated with the indicated gRNA.

FIG. 7 illustrates exemplary delivery strategies for gene editing in an animal (e.g., a human or a mouse model of HD) using either a (A) single-vector or (B) dual-vector approach.

### DETAILED DESCRIPTION OF THE DISCLOSURE

In some aspects, disclosed herein are compositions and methods for editing a poly-Q stretch within the HTT gene. In one aspect, compositions include polypeptides, (e.g., the Cas9 nuclease). In another aspect, the disclosure also provides for polynucleotides (e.g., guide RNAs and/or expression cassettes); polynucleotides encoding said polypeptides; vectors comprising such polynucleotides (e.g., AAV vectors comprising such expression cassettes); methods of making those vectors; recombinant AAV (rAAV) particles comprising such vectors; pharmaceutical compositions comprising the polypeptides, the polynucleotides, the vectors, and/or the rAAV particles disclosed herein; and methods of using the polypeptides, the polynucleotides, the vectors, the rAAV particles, and/or the pharmaceutical compositions disclosed herein.

### Definitions

Unless otherwise defined, all technical and scientific terms used herein have the same meaning as commonly understood by one of ordinary skill in the art. In case of conflict, the present document, including definitions, will control. Unless expressly stated to the contrary herein, any term, as used in this application, shall have the meaning set forth in this application.

As used herein, the terms “about” and/or “approximately” shall mean within an acceptable error range for the particular value as determined by one of ordinary skill in the art, which will depend in part on how the value is measured or determined (i.e., the limitations of the measurement system). For example, “about” can mean within 3 or more than 3 standard deviations, per the practice in the art. Alternatively, “about” can mean a range of up to 20%, preferably up to 10%, more preferably up to 5%, and more preferably still up to 1% of a given value. In certain aspects, the term “about” refers to a range of values that fall within 20%, 19%, 181%, 17%, 16%, 15%, 14%, 13′%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, particularly with respect to biological systems or processes, the term can mean within an order of magnitude, preferably within 5-fold, and more preferably within 2-fold, of a value.

The terms “Adeno-associated virus” or “AAV” as used interchangeably herein refer to any virus belonging to the Parvoviridae family (genus Dependovirus) that endemically infects humans and some other primate species. AAV is not currently known to cause disease and consequently causes a very mild immune response. In addition to the naturally occurring serotypes of the virus, these terms shall expressly include any and all “recombinant variants” (e.g., engineered versions) of an AAV virus, including, but not limited to, AAVs with RGD insertions (see, e.g., Manini, A., et al. Frontiers in Neurology, 12, 814174 (2022)). Additional non-limiting examples of contemplated recombinant AAV variants include AAVrh.74, MyoAAV variants (e.g., Myo AAV2 and MyoAAV4E), and AAV-MYO variants.

The term “Cas9” as used herein Cas9” refers to a Type II CRISPR-Associated nuclease protein that is the active enzyme for a CRISPR-Cas9 system. “nCas9” refers to a Cas9 that has one of the two nuclease domains inactivated, i.e., either the RuvC or HNH domain. nCas9 is capable of cleaving only one strand of target DNA (a “nickase”). The term “Cas9” refers to an RNA-guided double-stranded DNA-binding nuclease protein or nickase protein, or a variant thereof. Herein, “Cas9” refers to both naturally occurring and recombinant Cas9 proteins. A wildtype Cas9 nuclease has two functional domains, e.g., RuvC and HNH, that cut different DNA strands. Cas9 enzymes described herein can comprise a HNH or HNH-like nuclease domain and/or a RuvC or RuvC-like nuclease domain. Cas9 can induce double-strand breaks in genomic DNA (e.g., a targeted gene) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species. In specific embodiments, the Cas9 protein is derived from Staphylococcus aureus.

The terms “coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

The terms “complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “comprise(s),” “include(s),” “having,” “has,” “contain(s),” and variants thereof, as used herein, are intended to be open-ended transitional phrases, terms, or words that do not preclude the possibility of additional acts, structures or components. The singular forms of articles, such as “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The terms “donor DNA,” “donor template,” and “repair template” as used interchangeably herein refer to a double-stranded DNA fragment or molecule that includes at least a portion of the gene of interest. The donor DNA may encode a full-functional protein or a partially-functional protein.

The term “efficiency,” as used herein in reference to genome editing, shall mean the rate at which a CRISPR system successfully edits a targeted polynucleotide, as measured by molecular assay, (e.g., ddPCR, Western blotting and/or gene sequencing) and is often expressed as a percentage of an unmodified control. Absolute editing efficiency may vary between two or more CRISPR systems due, wholly or in part, to the choice of a particular genetic sequence target, gRNA structure, chemical modifications of one or more nucleic acids in the system, choice of CRISPR nuclease, CRISPR nuclease amino acid substitutions, among other factors (see, e.g., Li, B., et al. Trends in Pharmacological Sciences, 41 (1), 55-65.) (2020).

The term “expression cassette” as used herein refers to a nucleic acid construct, generated recombinantly or synthetically, with a series of specified nucleic acid elements that permit transcription of a particular polynucleotide sequence in a competent host cell, such that a particular gene product (e.g., RNA or protein) is expressed. Expression of any gene product may be dependent upon presence of cellular factors or additional gene products from other expression cassettes. An expression cassette or vector may be part of a plasmid, viral genome, or nucleic acid fragment. Typically, an expression cassette or vector includes a polynucleotide to be transcribed, operably linked to a promoter.

The terms “frameshift” or “frameshift mutation,” which may be used interchangeably herein, refer to a type of genetic mutation wherein addition or deletion of one or more nucleotides causes a shift in the codon reading frame in the resultant mRNA, thereby altering the encoded amino acid sequence. Frameshifts may result in, for example, a missense mutation or a nonsense mutation (i.e., introduction of a premature stop codon).

The terms “functional” and “fully functional” as used herein describe protein that has biological activity. A “functional gene” refers to a gene transcribed to mRNA, which is translated to a functional protein.

The term “fusion protein” as used herein refers to a chimeric protein created through the joining of two or more genes that originally coded for separate proteins. The translation of the fusion gene results in a single polypeptide with functional properties derived from each of the original proteins.

The term “gene” as used herein refers to the segment of a DNA molecule that codes for a polypeptide chain (e.g., the coding region). In some embodiments, a gene is positioned by regions immediately preceding, following, and/or intervening the coding region that are involved in producing the polypeptide chain (e.g., regulatory elements such as a promoter, enhancer, polyadenylation sequence, 5′-untranslated region, 3′-untranslated region, or intron).

The terms “genetic construct” or “construct” as used herein refer to the nucleic acid molecules that comprise a nucleotide sequence encoding a protein. The coding sequence may be DNA or RNA and includes initiation and termination signals operably linked to regulatory elements, such as a promoter and polyadenylation signal capable of directing expression in the cells of the individual to whom the nucleic acid molecule is administered. As used herein, the term “expressible form” refers to genetic constructs that contain the necessary regulatory elements operably linked to a coding sequence that encodes a protein such that when present in the cell of an individual, the coding sequence will be expressed.

The term “genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, BMD, hemophilia, cystic fibrosis, Huntington's disease, familial hypercholesterolemia (LDL receptor defect), hepatoblastoma, Wilson's disease, congenital hepatic porphyria, inherited disorders of hepatic metabolism, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

The term “genome editing” as used herein refers to altering or modifying a mutant gene (i.e., one encoding a truncated protein or a non-functional protein), such that a full-length or partially full-length functional protein is expressed. Such activity may alternatively be considered “correcting” or “restoring” a mutant gene's functionality and may include replacing or excising an aberrant region of the mutant gene or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site, or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include deleting a non-essential or aberrant gene segment by the simultaneous action of two nucleases on the same DNA strand. Genome editing additionally refers to modulating expression of a gene as result of altering a genetic sequence (e.g., knocking out a gene, including a mutant gene or a normal gene). Genome editing may be used to treat disease caused by a mutant gene or to enhance repair of tissues by changing expression and/or sequence of a gene product of interest.

The terms “guide RNA” or “gRNA,” which may be used interchangeably herein, refer to one or more RNA molecules, preferably a synthetic RNA molecule, that comprise the RNA component of a CRISPR system (e.g., a CRISPR-Cas9 system) that guides a CRISPR-associated nuclease (e.g., Cas9) to a target polynucleotide or targeted gene. A gRNA is comprised of a targeting sequence and scaffold sequence. In some embodiments, the gRNA is a single-guide RNA (sgRNA). In some embodiments, the gRNA is composed of a crRNA and tracrRNA molecule. A sgRNA can be administered or formulated, e.g., as a synthetic RNA, or as a nucleic acid comprising a sequence encoding the gRNA, which is then expressed in one or more target cells. As would be evident to one of ordinary skill in the art, various tools may be used to design and/or optimize the sequence of a gRNA, for example, to increase the specificity and/or precision of genomic editing. In general, an ideal gRNA has a high predicted on-target efficiency and low off-target efficiency based on any of the available web-based tools. Candidate gRNAs may be further assessed by manual inspection and/or experimental screening. Examples of web-based tools include, without limitation, CRISPR seek, CRISPR Design Tool, Cas-OFFinder, E-CRISP, ChopChop, CasOT, CRISPR direct, CRISPOR, BREAKING-CAS, CrispRGold, and CCTop (Safari, et al. Current Pharma. Biotechol. (2017) 18 (13)). Such tools are also described, for example, in PCT Publication No. WO2014093701A1 and Liu, et al., “Computational approached for effective CRISPR guide RNA design and evaluation”, Comput Struct Biotechnol J., 2020; 18:35-44, each of which is incorporated by reference herein in its entirety for all purposes.

The terms “homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including targeted addition of whole genes. If a donor template is provided along with a CRISPR-Cas9 gene editing system, then the cellular machinery will repair the break by homologous recombination, which is enhanced several orders of magnitude in the presence of DNA cleavage. When the homologous DNA piece is absent, non-homologous end joining may take place instead.

The term “huntingtin” as used herein refers to the protein product of the HTT gene (NCBI Gene ID: 3064) e.g., NCBI Protein Accession No.: NP_001375421.1; UniProt: P11532. Huntingtin is a protein localized to the central nervous system (CNS) and has been observed to be expressed in neurons and glial cells. The “huntingtin gene” or “HTT gene” as used interchangeably herein is 180 kilobases in length and is located at locus 4p16.3 (see, e.g., NCBI Reference NG_009378.1). The primary transcript measures about 13,500 bases in length. Sixty-seven exons code for the huntingtin protein, which is composed of more than 3100 amino acids.

The terms “Huntington's Disease,” “HD,” or “Huntington's chorea,” which may be used interchangeably herein, refer to a genetic disorder that results in progressive degeneration of motor neuron function as a result of aberrant huntingtin function. Onset typically occurs between ages 30 and 50 and is invariably lethal.

The term “identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

The term “mutant gene” or “mutated gene” as used interchangeably herein refers to a gene that has undergone a detectable mutation. A mutant gene has undergone a change, such as the loss, gain, or exchange of genetic material, which affects the normal transmission, expression, and/or functionality of the gene. A “disrupted gene” as used herein refers to a mutant gene that has a mutation that causes a premature stop codon. The disrupted gene product is truncated relative to a full-length undisrupted gene product.

The term “neuron” as used herein refers to a differentiated cell capable of transmitting signals (e.g., action potentials) throughout the nervous system of a subject. Subsets of neurons (e.g., motor neurons) enervate, and thereby control, particular functions (e.g., motor functions). Dysregulation of one or more subsets can result in involuntary activity associated with the functions under their control. Additionally, neurons share a microenvironment with other nervous system cells, such as glial cells and/or astrocytes.

The term “neurological condition” as used herein refers to a condition related to the central nervous system (CNS) and/or peripheral nervous system (PNS) of a subject. Non-limiting examples of neurological conditions include Huntington's Disease, Alzheimer's Disease, Amyotrophic lateral sclerosis (ALS), multiple sclerosis, and Parkinson's Disease.

The terms “non-homologous end joining” or “NHEJ” as used herein refer to a cell-mediated DNA double-strand repair process that directly ligates the broken ends without the need for a homologous template. This template-independent re-ligation repair process is stochastic and error-prone, such that random micro-insertions and micro-deletions (indels) are regularly introduced at the DNA breakpoint. This method may be used to intentionally disrupt, delete, or alter the reading frame of targeted polynucleotide sequences in a subject's genome. NHEJ typically uses short homologous DNA sequences called microhomologies to guide repair. These microhomologies are often present in single-stranded overhangs at the end of double-strand breaks. When the overhangs are perfectly compatible, NHEJ usually repairs the break accurately; however, imprecise repair leading to loss of nucleotides may also occur and is much more common when the overhangs are not compatible.

The term “normal gene” as used herein refers to a gene that has not undergone a change, such as a loss, gain, or exchange of genetic material. The normal gene undergoes normal gene transmission and gene expression and is sufficiently functional to not cause symptomatic disease. For the avoidance of doubt, wildtype genes and asymptomatic variants of a wildtype gene (e.g., those exhibiting natural variability in the poly-Q stretch or those containing single-nucleotide polymorphisms (SNPs)), are herein considered normal genes.

The term “nuclease-mediated NHEJ” as used herein refers to NHEJ that is initiated after a nuclease, such as a Cas9 protein, induces a double-stranded DNA break.

The terms “nucleic acid,” “oligonucleotide” or “polynucleotide” as used herein refer to deoxyribonucleotides or ribonucleotides and polymers thereof in either single- or double-stranded form and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs). Any combinations of bases including uracil, adenine, thymine, cytosine, guanine, inosine, xanthine hypoxanthine, isocytosine and isoguanine are expressly contemplated by this application.

The term “operably linked” as used herein means that expression of a gene is under the control of a promoter or regulatory element with which it is spatially connected. For example, a promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term “partially functional” as used herein describes a protein that is encoded by a mutant gene and has less biological activity than a fully functional protein but more than a non-functional protein.

The term “poly-Q stretch,” as used herein in reference to the HTT gene, shall refer to the variable region within exon 1 of the huntingtin gene, wherein natural variability in the number of CAG repeats is observed. “Natural variability” in terms of CAG repeats is defined herein as any number between 9 and 35 repeats. “Pathogenic expansion” of the poly-Q stretch is accordingly defined as more than 35 such repeats.

The terms “promoter or “promoter element,” which may be used interchangeably, refer to a nucleotide sequence that assists with controlling expression of a coding sequence. Generally, promoters are located 5′ (i.e., upstream) of the translation start site of a gene. However, in certain embodiments, a promoter element may be located within an intron sequence, or 3′ of the coding sequence. A promoter may be derived from sources including viral, bacterial, fungal, plants, insects, and animals. In some embodiments, one of a plurality of well-characterized promoter elements is used with a vector described herein. Non-limiting examples of well-characterized promoter elements include a SV40 early promoter, a SV40 late promoter, a human U6 (hU6) promoter, a CMV early promoter, a β-actin promoter, and a JeT promoter. In some embodiments, the promoter is a constitutive promoter, which drives substantially constant expression of the target protein. In other embodiments, the promoter is tissue-specific promoter, which drives expression of the target protein in response to presence in a particular tissue or cell type. Non-limiting examples of cell-specific promoters include an astrocyte-specific promoter (e.g., a GFAP promoter), and a neuron specific promoter (e.g., a methyl CpG binding protein 2 (MeCP2) promoter).

A promoter may comprise one or more transcriptional regulatory elements to further enhance expression and/or to alter the spatial expression and/or temporal expression of the same. A promoter may also comprise distal enhancer or repressor elements, which may be located as much as several thousand base pairs from the start site of transcription.

The terms “protospacer,” “targeting sequence” or “crRNA sequence,” which may be used interchangeably refer to a component of a functional gRNA in a CRISPR system that has complementarity to a targeted polynucleotide or targeted gene.

The terms “Protospacer Adjacent Motif” or “PAM,” which may be used interchangeably herein, refer to the region of a targeted gene or targeted polynucleotide sequence that is recognized and bound by a CRISPR-associated (Cas) protein, such as Cas9. In some embodiments, the PAM is no more than 1, 2, 3, 4, 5, 6, 7, 8, 9, 10, 11, 12, 13, 14, 15 or 20 bases from a protospacer sequence. Naturally occurring Cas9 molecules recognize specific PAM sequences. It is understood that PAMs may be degenerate in nature such that multiple sequences are recognized by a particular protein (e.g., NGG for SpCas9 or NNGRRV/NNGRRT for SaCas9, wherein N means any nucleotide and R means any purine nucleotide, and V means any one of guanine, cytosine, and adenine). In some embodiments, the PAM is NNGRRV. In other embodiments, the PAM is NNGRRT.

The term “regulatory element” as used herein refers to nucleotide sequences, such as promoters, enhancers, terminators, polyadenylation sequences, introns, and the like, that provide for the expression of a coding sequence in a cell or otherwise control said expression.

The terms “subject” or “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate-such as, a monkey (e.g., a cynomolgus or rhesus monkey), a chimpanzee,—and a human). In some embodiments, the subject may be a human or a non-human. The subject or patient may be undergoing other forms of treatment.

The terms “target[ed] gene” or “target[ed] polynucleotide” as used herein refer to any nucleotide sequence encoding a known or putative gene product. The target gene may be a mutated gene involved in a genetic disease. In certain embodiments, the target gene is a human huntingtin gene. In certain embodiments, the target gene is a mutant human huntingtin gene.

The term “target region” as used herein refers to the region of the target gene to which the CRISPR/Cas9-based gene editing system is designed to bind and cleave. In some embodiments, the target region is complementary to the protospacer sequence.

The term “variant” as used herein encompasses, but is not limited to, proteins (including fusion proteins) which comprise an amino acid sequence that differs from the amino acid sequence of a reference protein by way of one or more substitutions, deletions and/or additions at certain positions within or adjacent to the amino acid sequence of the reference protein. A variant may comprise one or more conservative substitutions in its amino acid sequence as compared to the amino acid sequence of a reference protein. Conservative substitutions may involve, e.g., the substitution of similarly charged or uncharged amino acids. A variant retains the biological activity ascribed to the reference protein. Regarding nucleic acids encoding proteins, a variant may comprise one or more conservative substitutions in its sequence as compared to the sequence of a reference nucleic acid. Conservative nucleic acid substitutions may involve substitution at positions that do not alter the resultant encoded amino acid sequence.

The term “vector” as used herein refers to any vehicle used to transfer a nucleic acid (e.g., encoding a CRISPR-Cas9 system construct) into a host cell. In some embodiments, a vector includes a replicon, which functions to replicate the vehicle, along with the target nucleic acid. Non-limiting examples of vectors useful for therapeutic purposes include plasmids, phages, cosmids, artificial chromosomes, and viruses, which function as autonomous units of replication in vivo. In some embodiments, the vector is a viral vehicle for introducing a target nucleic acid (e.g., a CRISPR-Cas9 system construct). Many modified eukaryotic viruses useful for genetic construct delivery are known in the art. For example, adeno-associated viruses (AAVs) are particularly well-suited for use in human gene therapy because humans are a natural host for the virus, the native viruses are not known to contribute to any diseases, and the viruses illicit a mild immune response. In certain embodiments, the vector is a lipid nanoparticle.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

### Genetic Constructs For Genome Editing of The Huntingtin Gene

Provided herein are genetic constructs for genome editing, genomic alteration, and/or altering gene expression of a huntingtin gene. The huntingtin gene may be a human huntingtin gene. The genetic constructs include at least one gRNA that targets a huntingtin gene sequence(s). The at least one gRNA may target a mouse, a human and/or a rhesus monkey huntingtin gene sequence. The at least one gRNA may be compatible with SaCas9. The at least one gRNA may be compatible with KKH-SaCas9. The at least one gRNA may be compatible with SpCas9. All gRNAs disclosed herein may be included in a CRISPR/Cas9-based gene editing system, including systems that use SaCas9, KKH-SaCas9 or SpCas9, to an exon of the human huntingtin gene. The gRNAs disclosed herein, which may be included in a CRISPR/Cas9-based gene editing system, can cause genomic alterations of the human huntingtin gene in order to disrupt expression of huntingtin protein in cells from HD patients.

### Huntingtin and Huntington's Disease Biology

The huntingtin gene is widely expressed and is required for normal development. It is expressed as two alternatively polyadenylated forms displaying different relative abundance in various fetal and adult tissues. The larger transcript is approximately 13.7 kb and is expressed predominantly in adult and fetal brain, whereas the smaller transcript of approximately 10.3 kb is more widely expressed. Acquired genetic defects in this gene that lead to Huntington's Disease (HD) may confer a new property on the mRNA or alter the function of the huntingtin protein.

Without wishing to be bound to any particular theory, HD results from pathogenic expansions in the poly-Q stretch of the huntingtin gene, resulting in aggregation of the protein. HD patients are typically diagnosed during adulthood following the onset of any combination of cognitive, motor, and psychiatric symptoms. Such symptoms worsen as the disease progresses, ultimately resulting in the need for round-the-clock care prior to lethality, typically within 10 to 20 years from diagnosis.

### CRISPR Gene-Editing Systems

In one aspect, compositions disclosed herein, including one or more presently disclosed genetic constructs, can mediate highly efficient gene editing of the huntingtin gene. In some embodiments, a presently disclosed genetic construct can ablate huntingtin protein expression in cells from HD patients. A presently disclosed genetic construct may be transfected into human HD cells and mediate efficient gene modification and conversion to disrupt the reading frame by NHEJ-based genome editing and/or HDR results in a non-functional huntingtin protein. Without wishing to be bound by any particular theory, disrupting protein expression, as detected in a bulk population of CRISPR/Cas9-based gene editing system-treated cells, may be concomitant with treatment of HD by halting pathogenic aggregation in neurons.

In some embodiments, a presently disclosed genetic construct can restore huntingtin protein expression in cells from HD patients through targeted modification of any one of exons 1, 2, and 3 of the human huntingtin gene. Without wishing to be bound to a particular theory, excision of pathogenic expansions of the poly-Q stretch from the huntingtin transcript can effectively treat HD patients. Such interventions are suited for permanent correction by NHEJ-based genome editing and/or HDR. A presently disclosed genetic construct may be transfected into human HD cells and mediate efficient gene modification and conversion to a non-pathogenic protein (e.g. one not including the poly-Q stretch). Non-pathogenic protein restoration may be concomitant with detection in a bulk population of CRISPR/Cas9-based gene editing system-treated cells.

A presently disclosed genetic construct may encode a CRISPR/Cas9-based gene editing system that is specific for a huntingtin gene. “Clustered Regularly Interspaced Short Palindromic Repeats” or “CRISPR,” as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial genomes contain a combination of CRISPR-associated (Cas) genes as well as non-coding RNA elements capable of programming the specificity of the CRISPR-mediated nucleic acid cleavage.

Short segments of foreign DNA, called spacers, are incorporated into the genome between CRISPR repeats, and serve as a ‘memory’ of past exposures. Cas9 forms a complex with the 3′ end of the gRNA and the protein-RNA pair recognizes its genomic target by complementary base pairing between the 5′ end of the gRNA sequence and a predefined DNA sequence of about 20 base paires, known as the protospacer. This complex is directed to homologous loci of a pathogenic DNA via regions encoded within the crRNA, protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the recognition sequence of the expressed gRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and Ill effector systems) are known. The Type II effector system carries out targeted DNA double-strand break in four sequential steps, using a single effector enzyme, Cas9, to cleave dsDNA. Compared to the Type I and Type Ill effector systems, which require multiple distinct effectors acting as a complex, the Type II effector system may function in alternative contexts such as eukaryotic cells. The Type II effector system consists of a long pre-crRNA, which is transcribed from the spacer containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarity between a protospacer sequence in the target DNA and the remaining spacer sequence in the crRNA. Cas9 mediates cleavage of target DNA if a correct protospacer-adjacent motif (PAM), a short sequence recognized by the Cas9 nuclease that is required for DNA cleavage, is also present at the 3′ end of the protospacer. For protospacer targeting, the sequence must be immediately followed by the PAM.

Different Type II systems have different PAM requirements. For instance, a CRISPR system derived from S. pyogenes may have the PAM sequence for its Cas9 (SpCas9) as 5′-NGG-3′ and/or 5′-NRG-3′ (where R is either A or G).

A unique capability of the CRISPR/Cas9-based gene editing system is the straightforward ability to simultaneously target multiple distinct genomic loci by co-expressing a single Cas9 protein with two or more gRNAs. For example, the Streptococcus pyogenes Type II system naturally prefers to use an NGG sequence (where N can be any nucleotide) but also accepts other PAM sequences, such as “NAG” in engineered systems (Hsu et al., Nature Biotechnology (2013) doi: 10.1038/nbt.2647). Similarly, the Cas9 derived from N. meningitidis (NmCas9) normally has a native PAM of NNNNGATT but has activity across a variety of PAMs, including a highly degenerate NNNNGNNN PAM (Esvelt et al. Nature Methods (2013) doi: 10.1038/nmeth2681).

A Cas9 molecule derived from S. aureus recognizes the sequence motif NNGRRT (R=A or G) and directs cleavage of a target nucleic acid sequence 1 to 10, e.g., 3 to 5, bp upstream from that sequence. In certain embodiments, a Cas9 molecule of S. aureus recognizes the PAM sequence motif NNGRRN (R=A or G) and directs cleavage of a target nucleic acid sequence 1 to 10 (e.g., 3 to 5) bp upstream from that sequence. In certain embodiments, a Cas9 molecule derived from S. aureus recognizes the sequence motif NNGRRT (R=A or G) and directs cleavage of a target nucleic acid sequence 1 to 10 (e.g., 3 to 5) bp upstream from that sequence. In certain embodiments, a Cas9 molecule derived from S. aureus recognizes the sequence motif NNGRRV (R=A or G) and directs cleavage of a target nucleic acid sequence 1 to 10 (e.g., 3 to 5) bp upstream from that sequence.

In one aspect, CRISPR and CRISPR-associated RNA-guided nuclease-related methods, components and compositions of the disclosure (hereafter, CRISPR/Cas systems) minimally require at least one isolated or non-naturally occurring protein component (e.g., a Cas protein) and at least one isolated or non-naturally occurring nucleic acid component (e.g., a guide RNA (gRNA)) to effectuate augmentation of a ‘nucleic acid sequence (e.g., genomic DNA).

In some embodiments, a CRISPR/Cas system effectuates the alteration of a targeted gene or locus in a eukaryotic cell by effecting an alteration of the sequence at a target position (e.g., by creating an insertion or deletion (collectively, an indel) resulting in loss-of-function of (i.e., knocking out) the affected gene or allele; e.g., a nucleotide substitution resulting in a truncation, nonsense mutation, or other type of loss-of-function of an encoded gene product; a of loss-of-function of, for example, an encoded gene product; e.g., loss-of-function of the encoded mRNA or protein by a single nucleotide, double nucleotide, or other frame-shifting deletion, or a deletion resulting in a premature stop codon; or an insertion resulting in a truncation, nonsense mutation, or other type of loss-of-function of an encoded gene product, such as the encoded mRNA or protein; e.g., a single nucleotide, double nucleotide, or other frame-shifting insertions, or an insertion resulting in a premature stop codon. The present disclosure provides for the alteration of an exon (e.g., exon 1 or exon 3) of the HTT gene in a patient with HD by generating an indel that ablates expression of a pathogenic huntingtin protein.

### CRISPR Nucleases

In one aspect, CRISPR/Cas systems effectuate changes to the sequence of a nucleic acid through nuclease activity. For example, in the case of genomic DNA, the nuclease-guided by a protein-associated exogenous nucleic acid that locates a target position within a targeted gene or locus by sequence complementarity with a portion of the protein-associated nucleic acid (e.g., a protospacer, a CRISPR RNA (crRNA) or a complementary component of a synthetic single guide RNA (sgRNA))—cleaves the genomic DNA upon recognition of particular, nuclease-specific motif called the protospacer adjacent motif (PAM). See generally, Collias, D., & Beisel, C. L. (2021). Nature Communications, 12 (1), 1-12.

Nuclease activity (i.e., cleavage) induces a double-strand break (DSB) in the case of genomic DNA. Endogenous cellular mechanisms of DSB repair, namely non-homologous end joining (NHEJ), microhomology-mediated end joining (MMEJ), and homologous recombination, result in erroneous repair at a given target position with some calculable frequency as a result of interference from said components of the CRISPR/Cas system, thereby introducing substitutions or indels into the genomic DNA. See generally Scully, R., et al. (2019). Nature Reviews Molecular Cell Biology, 20 (11), 698-714. At some frequency, these indels and/or substitutions may result in frameshifts, nonsense mutations (i.e., early stop codons) or truncations that impact the availability of gene products, such as mRNA and/or protein. In certain embodiments, the CRISPR/Cas system may induce a homology-directed repair (HDR) mechanism leading to insertions of non-random sequences as part of the system along with the nuclease and gRNA. See Bloh, K., & Rivera-Torres, N. (2021). International Journal of Molecular Sciences, 22 (8), 3834.

In general, the minimum requirements of the CRISPR/Cas system will be dependent upon the nuclease (i.e., Cas protein) provided therewith. To this extent, these bacterially derived nucleases have been functionally divided into Types I, III, and V, which all fall into Class 1 and Types II, IV, and VI that are grouped into Class 2.

### Class 1 CRISPR/Cas Systems:

The exact components, compositions, and methods for effectuating a change in a targeted nucleic acid sequence using a Class 1 CRISPR/Cas system will vary but should minimally include: a nuclease (selected from at least Types I, and III), at least one guide RNA selected from 1) sgRNA or 2) a combination of crRNA and tracrRNA. These CRISPR/Cas systems have been categorized together as Class 1 CRISPR/Cas systems due to their similarities in requirements and mode of action within a eukaryotic cell. To this end, compositions, components, and methods among Class 1 constituents may be considered functionally interchangeable, and the following details, provided merely for exemplary purposes, do not represent an exhaustive list of class members.

Cas3 is the prototypical Type I DNA nuclease that functions as the effector protein as part of a larger complex (the Cascade complex comprising Cse1, Cse2), that is capable of genome editing. See generally He, L., et al. (2020). Genes, 11 (2), 208. Unlike other CRISPR/Cas systems, Type I systems localize to the DNA target without the Cas3 nuclease via the Cascade complex, which then recruits Cas3 to cleave DNA upon binding and locating the 3′ PAM. The Cascade complex is also responsible for processing crRNAs such that they can be used to guide it to the target position. Because of this functionality, Cascade has the ability to process multiple arrayed crRNAs from a single molecule (see, Luo, M. (2015). Nucleic Acids Research, 43 (1), 674-681). As such, Type I system may be used to edit multiple targeted genes or loci from a single molecule.

Because the natural Cas3 substrate is ssDNA, its function in genomic editing is thought to be as a nickase; however, when targeted in tandem, the resulting edit is a result of blunt end cuts to opposing strands to approximate a blunt-cutting endonuclease, such as Biology, 20 (8), 490-507.

Like Type I nucleases, the Type III system relies upon an complex of proteins to effect nucleic acid cleavage. Particularly, Cas 10 possesses the nuclease activity to cleave ssDNA in prokaryotes. See Tamulaitis, G. Trends in Microbiology, 25 (1), 49-61. Interestingly, this CRISPR/Cas system, native to archaea, exhibits dual specificity and targets both ssDNA and ssRNA. Aside from this change, the system functions much like Type I in that the crRNA targets an effector complex (similar to Cascade) in a sequence-dependent manner. Similarly, the effector complex processes crRNAs prior to association. The dual nature of this nuclease makes its applications to genomic editing potentially more powerful, as both genomic DNA and, in some cases, mRNAs with the same sequence may be targeted to silence particular targeted genes.

### Class 2 CRISPR/Cas Systems:

The exact components, compositions, and methods for effectuating a change in a targeted nucleic acid sequence using a Class 2 CRISPR/Cas system will vary but should minimally include: a nuclease (selected from at least Types II, and V), at least one guide RNA selected from 1) sgRNA or 2) a combination of crRNA and tracrRNA. These CRISPR/Cas systems have been categorized together as Class 2 CRISPR/Cas systems due to their similarities in requirements and mode of action within a eukaryotic cell. To this end, compositions, components, and methods among Class 2 constituents may be considered functionally interchangeable, and the following details, provided merely for exemplary purposes, do not represent an exhaustive list of class members.

Type II nucleases are the best characterized CRISPR/Cas systems, particularly the canonical genomic editing nuclease Cas9 (see Table 4). Multiple Cas9 proteins, derived from various bacterial species, have been isolated. The primary distinction between these nucleases is the PAM, a required recognition site within the targeted dsDNA. After association with a gRNA molecule, the crRNA (or targeting domain of a sgRNA) orients the nuclease at the proper position, but the protein's recognition of the PAM is what induces a cleavage event near that site, resulting in a blunt DSB. have similarly been reported. These range from Cas9 with enhanced specific (i.e., less off-target activity), such as espCas9. Others have been catalytically modified via point mutations in the RuvC (e.g., D10A) and HNH (e.g., H840A) domains such that they induce only single-strand breaks (i.e., Cas9 nickases). See Frock, R. et al. (2015). Nature Biotechnology, 33 (2), 179-186. These variants, collectively referred to herein as enhanced specificity Cas9 variants (spCas9), have also been shown to be less error-prone in editing. Such mitigation of off-target effects becomes paramount when selecting for a desired insertion (i.e., a knock in mutation, in which a desired nucleotide sequence is introduced into a target nucleic acid molecule) rather than a deletion. Indeed, less off-target effects may aid in the preferred DNA repair mechanism (HDR, in most instances for knock in mutations). See generally, Naeem, M., et al. (2020). Cells, 9 (7), 1608.

Additional exemplary further engineered variants of canonical Cas proteins (e.g., mutants, chimeras, and include the following (which are hereby incorporated by reference): WO2015035162A2, WO2019126716A1, WO2019126774A1, WO2014093694A1, and WO2014150624A1. For the avoidance of doubt, spCas9 collectively refers to any one of the group consisting of espCas9 (also referred to herein as ESCas9 or esCas9), HFCas9, PECas9, arCas9, and KKH-SaCas9.

Like the canonical Cas9 systems, Type V nucleases only require a synthetic sgRNA with a targeting domain complementary to a genomic sequence to carry out genomic editing. These nucleases contain a RuvC domain but lack the HNH domain of Type II nucleases. Further, Cas12, for example, leaves a staggered cut in the dsDNA substrate distal to the PAM, as compared to Cas9's blunt cut next to the PAM. Both Cas12a, also known as Cpf1, and Cas12b, also known as C2c1 (see Table 4), act as part of larger complex of two gRNA-associated nucleases that act on dsDNA as quaternary structure nicking each strand simultaneously (see Zetsche B, et al. Cell. 2015; 163 (3): 759-771; see also Liu L, et al. Mol Cell. 2017; 65 (2): 310-322). Additionally, Cas12b (C2c1) is a highly accurate nuclease with little tolerance for mismatches. See Yang H, et al. Cell. 2016; 167 (7): 1814-1828.e12.

### CRISPR Guide RNAs

In one aspect, the CRISPR/Cas system of the present disclosure further provides a gRNA molecule (e.g., an isolated or non-naturally occurring RNA molecule) that interacts with a Cas protein. In certain embodiments, the gRNA is an sgRNA, in which the crRNA (i.e., the targeting domain or complementary region) comprises a nucleotide sequence selected from SEQ ID NOs: 1-120 to target a human HTT gene. In certain embodiments, the targeting domain is a crRNA that is provided to a eukaryotic cell with tracrRNA, which acts as a scaffold through interactions with both the crRNA and a Cas protein. In some embodiments, the system is further, optionally, comprised of an oligonucleotide—an HDR template with homology to either side of the target position (see Bloh, K., & Rivera-Torres, N, at 3836).

In some embodiments, the crRNA of the gRNA molecule is configured to orient an associated nuclease such that a cleavage event, (e.g., a double strand break or a locus, thereby facilitating an alteration in the nucleic acid sequence. In some embodiments, the crRNA is 20 nucleotides in length. In some embodiments, the crRNA is 21 nucleotides in length. In some embodiments, the crRNA is 14, 15, 16, 17, 18, 19, 20, 21, 22, 23, 24, 25, 26, 27, 28, 29, or 30 nucleotides in length. In some embodiments, the crRNA orients the nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. The double-strand or single-strand break may be positioned upstream or downstream of a target position, and either within or upstream a functional domain cluster within the targeted gene.

In certain embodiments, a second gRNA molecule, comprising a second crRNA orients a second associated nuclease such that a cleavage event occurs sufficiently close to a target position, in the targeted gene or locus, thereby facilitating an alteration in the nucleic acid sequence. In an embodiment, the second gRNA molecule targets the same targeted gene or locus as the first gRNA molecule. In other embodiments, the second gRNA molecule targets a different targeted gene or locus as the first gRNA molecule. In some embodiments, the second crRNA is 20 nucleotides in length. In some embodiments, the second crRNA is 14, 15, 16, 17, 18, 19, 20, 21, 22, 23, 24, 25, 26, 27, 28, 29, or 30 nucleotides in length.

In some embodiments, the second crRNA orients the nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. The double-strand or single-strand break, may be positioned upstream or downstream of a target position, and either within or upstream a functional domain cluster within the targeted gene.

In some embodiments, the crRNAs of a first and second gRNA molecules are configured such that a cleavage event is positioned, independently for each of the gRNA molecules, within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of the respective target position. In certain embodiments, the first and second gRNA molecules alter the targeted nucleic acid sequences simultaneously. In certain embodiments, the first and second gRNA molecules alter the targeted nucleic acid sequences sequentially. strand break, positioned by the crRNAs of a first and second gRNA molecule, respectively. For example, the crRNAs may orient the associated nucleases such that a cleavage event, (e.g., the two single-strand breaks), are positioned within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. In some embodiments, the crRNA of a first and second gRNA molecules are configured to orient associated nucleases such that, for example, two single-strand breaks occurs at the same target position, or within 1, 2, 3, 4, 5, 10, 15, 20, 25, 30, 35, 40, 45, 50 nucleotides of one another, on opposing strands of genomic DNA, thereby essentially approximating a double strand break.

In some embodiments, a nucleic acid encodes a crRNA comprising a protospacer sequence selected from any one of the sequences listed in Table 1. In some embodiments, a nucleic acid also comprises a polynucleotide of an associated PAM selected from any one of the sequences listed in Table 1. In some embodiments, a nucleic acid encodes a gRNA molecule comprising a crRNA sequence selected from SEQ ID NOs: 1-40. In some embodiments, a nucleic acid further encodes a second gRNA molecule selected from SEQ ID NOs: 1-40. In some embodiments, a nucleic acid further encodes a third and/or a fourth gRNA molecule selected from SEQ ID NOs: 1-40.

In some embodiments, a nucleic acid encodes a crRNA comprising a protospacer sequence selected from any one of the sequences listed in Table 2. In some embodiments, a nucleic acid also comprises a polynucleotide of an associated PAM selected from any one of the sequences listed in Table 2. In some embodiments, a nucleic acid encodes a gRNA molecule comprising a crRNA sequence selected from SEQ ID NOs: 41-80. In some embodiments, a nucleic acid further encodes a second gRNA molecule selected from SEQ ID NOs: 41-80. In some embodiments, a nucleic acid further encodes a third and/or a fourth gRNA molecule selected from SEQ ID NOs: 41-80.

In some embodiments, a nucleic acid encodes a crRNA comprising a protospacer sequence selected from any one of the sequences listed in Table 3. In some embodiments, a nucleic acid also comprises a polynucleotide of an associated PAM selected from any one of the sequences listed in Table 3. In some embodiments, a nucleic acid encodes a gRNA molecule comprising a crRNA comprising a protospacer sequence selected from SEQ ID NOs: 81-120. In some embodiments, a nucleic acid further encodes a second gRNA molecule comprising a protospacer sequence selected from SEQ ID NOs: 81-120. In some embodiments, a nucleic acid further encodes a third and/or a fourth gRNA molecule comprising a protospacer sequence selected from SEQ ID NOs: 81-120.

In certain embodiments, a nucleic acid may comprise (a) a sequence encoding a first gRNA molecule, comprising a crRNA comprising a protospacer sequence that is complementary with a target position in the targeted gene or locus, and (b) a sequence encoding an RNA-guided nuclease (e.g., Cas9). Optionally, (c), (d) and (e) are sequences encoding a second, a third and a fourth gRNA molecule, respectively. In some embodiments, one or more gRNAs target the same gene or locus. In some embodiments, (a) and (b), are encoded within the same nucleic acid molecule (i.e., the same vector, the same viral vector, the same adeno-associated virus (AAV) vector). In some embodiments, (a) and (b) are encoded within the separate nucleic acid molecules (i.e., separate vector, separate viral vectors, separate adeno-associated virus (AAV) vectors).

The CRISPR/Cas9 gene-editing system includes at least one gRNA molecule. The gRNA provides the targeting of a CRISPR/Cas9 gene-editing system. In some embodiments, the gRNA is a sgRNA molecule. The sgRNA is a fusion of two noncoding RNAs: a crRNA and a tracrRNA. The sgRNA may target any desired DNA sequence by exchanging the sequence encoding a protospacer of about 20 bp, which confers targeting specificity through complementary base pairing with the desired DNA target. The gRNA mimics the naturally occurring crRNA: tracrRNA duplex involved in the Type II Effector system. This duplex, which may include, for example, a 42-nucleotide crRNA and a 75-nucleotide tracrRNA, acts as a guide for Cas9 to cleave a targeted polynucleotide. The CRISPR/Cas9 gene-editing system may include at least one gRNA, wherein each gRNA targets a different DNA sequence. The target DNA sequences may be overlapping. The target sequence or protospacer is followed by a PAM sequence at the 3′ end of the protospacer. Different Type II systems have differing PAM requirements. For example, the Streptococcus pyogenes Type II system uses an “NGG” sequence, where “N” can be any nucleotide. In some embodiments, the PAM sequence may be “NGG,”, where “N” can be any nucleotide. In some embodiments, the PAM sequence may be NNGRRT or NNGRRV.

The number of gRNA molecules encoded by a presently disclosed genetic construct (e.g., an AAV vector) can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different 1RNAs, at least 12 different gRNAs, at least 13 different gRNAs, at least 14 different gRNAs, at least i 5 different gRNAs, at least 16 different gRNAs, at least 17 different gRNAs, at least i 8 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules encoded by a presently disclosed genetic construct can be less than 50 gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, or less than 3 different gRNAs. The number of gRNAs encoded by a presently disclosed genetic construct can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs. In certain embodiments, the genetic construct (e.g., an AAV vector) encodes one gRNA molecule, a Cas9 molecule. In certain embodiments, a first genetic construct (e.g., a first AAV vector) encodes one gRNA molecule, i.e., a first gRNA molecule, and optionally a Cas9 molecule, and a second genetic construct (e.g., a second AAV vector) encodes one gRNA molecule, i.e., a second gRNA molecule, and optionally a Cas9 molecule.

The gRNA molecule comprises a targeting domain (also referred to as a targeting sequence or cRNA sequence), which is a complementary polynucleotide sequence of the target DNA sequence followed by a PAM sequence. The gRNA may comprise a “G” at the 5′ end of the targeting domain or complementary polynucleotide sequence. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. The targeting domain of a gRNA molecule may comprise less than a 40 base pair, less than a 35 base pair, less than a 30 base pair, less than a 25 base pair, less than a 20 base pair, less than a 19 base pair, less than a 18 base pair, less than a 17 base pair, less than a 16 base pair, less than a 15 base pair, less than a 14 base pair, less than a 13 base pair, less than a 12 base pair, less than a 11 base pair, or less than a 10 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The gRNA may target a region of the huntingtin gene (HTT). In certain embodiments, the gRNA can target at least one of exons, introns, the promoter region, the enhancer region, the transcribed region of the huntingtin gene. In certain embodiments, the gRNA molecule targets exon 1 of the human huntingtin gene. In certain embodiments, the gRNA molecule targets exon 3 of the human huntingtin gene. In certain embodiments, the gRNA molecule targets exon 4 of the human huntingtin gene. In certain embodiments, the gRNA molecule targets exon 5 of the human huntingtin gene. In certain embodiments, the gRNA molecule targets exon 6 of the human huntingtin gene. In certain embodiments, the gRNA molecule targets exon 7 of the human huntingtin gene. In certain embodiments, the gRNA molecule targets exon 8 of the human huntingtin gene. In certain embodiments, the gRNA molecule targets exon 9 of the human huntingtin gene. In certain embodiments, the gRNA molecule targets exon 12 of the human huntingtin gene. In certain embodiments, the gRNA molecule targets exon 13 of the human huntingtin gene. In certain embodiments, the gRNA molecule targets exon 15 of the human huntingtin gene. In certain embodiments, the gRNA molecule targets exon 16 of the human huntingtin gene. In certain embodiments, the gRNA molecule targets exon 17 of the human huntingtin gene. In certain embodiments, the gRNA molecule targets exon 18 of the human huntingtin gene. In certain embodiments, the gRNA molecule targets exon 19 of the human huntingtin gene. In certain embodiments, the gRNA molecule targets exon 21 of the human huntingtin gene. In certain embodiments, the gRNA molecule targets exon 22 of the human huntingtin gene. In certain embodiments, the gRNA molecule targets exon 23 of the human huntingtin gene. In certain embodiments, the gRNA molecule targets exon 24 of the human huntingtin gene. In certain embodiments, the gRNA molecule targets exon 25 of the human huntingtin gene. In certain embodiments, the gRNA molecule targets exon 26 of the human huntingtin gene. In certain embodiments, the gRNA molecule targets exon 28 of the human huntingtin gene. In certain embodiments, the gRNA molecule targets exon 29 of the human huntingtin gene. In certain embodiments, the gRNA molecule targets exon 33 of the human huntingtin gene. In certain embodiments, the gRNA molecule targets exon 37 of the human huntingtin gene. In certain embodiments, the gRNA molecule targets exon 38 of the human huntingtin gene. In certain embodiments, the gRNA molecule targets exon 39 of the human huntingtin gene. In certain embodiments, the gRNA molecule targets exon 40 of the human huntingtin gene. In certain embodiments, the gRNA molecule targets exon 41 of the human huntingtin gene. In certain embodiments, the gRNA molecule targets exon 42 of the human huntingtin gene. In certain embodiments, the gRNA molecule targets exon 43 of the human huntingtin gene. In certain embodiments, the gRNA molecule targets exon 44 of the human huntingtin gene. In certain embodiments, the gRNA molecule targets exon 45 of the human huntingtin gene. In certain embodiments, the gRNA molecule targets exon 46 of the human huntingtin gene. In certain embodiments, the gRNA molecule targets exon 47 of the human huntingtin gene. In certain embodiments, the gRNA molecule targets exon 48 of the human huntingtin gene. In certain embodiments, the gRNA molecule targets exon 49 of the human huntingtin gene. In certain embodiments, the gRNA molecule targets exon 50 of the human huntingtin gene. In certain embodiments, the gRNA molecule targets exon 52 of the human huntingtin gene. In certain embodiments, the gRNA molecule targets exon 53 of the human huntingtin gene. In certain embodiments, the gRNA molecule targets exon 56 of the human huntingtin gene. In certain embodiments, the gRNA molecule targets exon 58 of the human huntingtin gene. In certain embodiments, the gRNA molecule targets exon 60 of the human huntingtin gene. In certain embodiments, the gRNA molecule targets exon 61 of the human huntingtin gene. In certain embodiments, the gRNA molecule targets exon 62 of the human huntingtin gene. In certain embodiments, the gRNA molecule targets exon 63 of the human huntingtin gene. In certain embodiments, the gRNA molecule targets exon 65 of the human huntingtin gene. In certain embodiments, the gRNA molecule targets exon 66 of the human huntingtin gene. In some embodiments, a first gRNA and a second gRNA each target an exon of a human huntingtin gene such that huntingtin protein expression is reduced.

### CRISPR Methods Targeting The Huntingtin Gene

In one aspect, the CRISPR/Cas9 system of the present disclosure comprises a method for the use of at least one Cas protein derived from one or more of the following selected bacterial genera: Corynebacterium, Sutterella, Legionella, Treponema, Filifactor, Eubacterium, Streptococcus, Lactobacillus, Mycoplasma, Bacteroides, Flavobacterium, Spirochaeta, Azospirillum, Gluconacetobacter, Neisseria, Roseburia, Parvibaculum, Nitratifractor, Campylobacter, Pseudomonas, Streptomyces, Staphylococcus, Francisella, Acidaminococcus, Lachnospiraceae, Leptotrichia, and Prevotella. In some embodiments, the Cas protein is derived from Deltaproteobacteria or Planctomycetes bacterial species.

Some aspects of the present disclosure provide strategies, methods, compositions, and treatment modalities for altering a targeted sequence within a gene locus (e.g., altering the genomic sequence of a cell from a patient with HD) with an RNA-guided nuclease and one or more guide RNAs (gRNAs), resulting in deletion or insertion of one or more nucleotides within the targeted gene product.

In certain embodiments, any region of the HTT gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, exon 67, intron 1, intron 2, intron 3, intron 4, intron 5, intron 6, intron 7, intron 8, intron 9, intron 10, intron 11, intron 12, intron 13, intron 14, intron 15, intron 16, intron 17, intron 18, intron 19, intron 20, intron 21, intron 22, intron 23, intron 24, intron 25, intron 26, intron 27, intron 28, intron 29, intron 30, intron 31, intron 32, intron 33, intron 34, intron 35, intron 36, intron 37, intron 38, intron 39, intron 40, intron 41, intron 42, intron 43, intron 44, intron 45, intron 46, intron 47, intron 48, intron 49, intron 50, intron 51, intron 52, intron 53, intron 54, intron 55, intron 56, intron 57, intron 58, intron 59, intron 60, intron 61, intron 62, intron 63, intron 64, intron 65, intron 66, any intron/exon junction, the 3′ UTR, or polyadenylation signal) is targeted by an RNA-guided nuclease to alter the gene. In certain embodiments, the targeted gene encodes human huntingtin.

Disclosed herein are methods of genome editing in a subject. The genome editing may be in a neuron, astrocyte and/or glial cell of a subject. In some embodiments, the method may comprise administering to the neuron, astrocyte and/or glial cell of the subject, a system or composition for genome editing, as described above. The genome editing may include correcting a mutant gene or inserting a transgene. Correcting the mutant gene may include deleting, rearranging, or replacing the mutant gene. Correcting the mutant gene may include nuclease-mediated NHEJ or HDR.

Disclosed herein are methods of correcting a mutant gene (e.g., a mutant huntingtin gene, e.g., a mutant human huntingtin gene) in a cell and treating a subject suffering from a genetic disease, such as HD. The method can include administering, to a cell or a subject, a presently disclosed system or genetic construct (e.g., a vector) or a composition comprised thereof as described above. The method can comprise administering to the neuron, astrocyte, and/or glial cell of the subject a presently disclosed system or genetic construct (e.g., a vector) or a composition comprising the same for genome editing in said neuron, astrocyte, and/or glial cell, as described above. In some embodiments use of a presently disclosed system or genetic construct (e.g., a vector) or a composition comprising the same to deliver the CRISPR/Cas9-based gene editing system to a neuron, astrocyte, and/or glial cell may reduce huntingtin protein expression by at least 50%. The CRISPR/Cas9-based gene editing system may be used to introduce site-specific double strand breaks at targeted genomic loci. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to, among other possible repair pathways (e.g., homology-directed repair (HDR) or the non-homologous end joining (NHEJ) pathway), ligation without an excised stretch of genomic DNA.

Provided herein is genome editing with a CRISPR/Cas9-based gene editing system without a repair template, which can efficiently correct the reading frame and restore the expression of a functional protein involved in a genetic disease. The disclosed CRISPR/Cas9-based gene editing systems may involve using homology-directed repair or nuclease-mediated non-homologous end joining (NHEJ)-based correction approaches, which enable efficient correction in proliferation-limited primary cell lines that may not be amenable to homologous recombination or selection-based gene correction. This strategy integrates the rapid and robust assembly of active CRISPR/Cas9-based gene editing systems with an efficient gene editing method for the treatment of genetic diseases caused by mutations in nonessential coding regions that cause frameshifts, premature stop codons, aberrant splice donor sites, aberrant splice acceptor sites, or pathogenic expansions.

The present disclosure is directed to a method of treating a subject in need thereof. The method comprises administering to a tissue of a subject a presently disclosed system or genetic construct (e.g., a vector) or a composition comprising thereof, as described above. In certain embodiments, the method may comprise administering, to a neuron, astrocyte and/or glial cell of the subject, the presently disclosed system or genetic construct (e.g., a vector) or composition comprising thereof, as described above. In certain embodiments, the method may comprise administering to a vein of the subject the presently disclosed system or genetic construct (e.g., a vector) or composition comprising thereof, as described above. In certain embodiments, the subject is suffering from a genetic disease or neurological condition causing cognitive and/or motor impairment. For example, the subject may be suffering from Huntington's Disease (HD), as described above.

The method, as described above, may be used for correcting defects in the huntingtin gene by reducing huntingtin protein expression. In some aspects and embodiments, the present disclosure provides a method for reducing the effects (e.g., clinical symptoms/indications) of HD in a patient. In some aspects and embodiments, the disclosure provides a method for treating HD in a patient. In some aspects and embodiments, the disclosure provides a method for preventing HD in a patient. In some aspects and embodiments, the present disclosure provides a method for preventing further progression of HD in a patient.

### Pharmaceutical Compositions Targeting The Huntingtin Gene

Further disclosed herein are DNA targeting compositions that comprise genetic constructs. The DNA targeting compositions include at least one gRNA molecule (for example, two gRNA molecules) that targets a huntingtin gene (for example, a human huntingtin gene), as described above. In some embodiments, the at least one gRNA molecule can bind and recognize a genomic target region. In some embodiments, the target regions can be chosen immediately upstream of possible in-reading-frame mutations (e.g. a pathogenic expansion), such that insertions or deletions during the repair process maintain the huntingtin reading frames. In some embodiments, the target regions can be chosen immediately upstream of possible frameshift mutations, such that insertions or deletions during the repair process restore the huntingtin reading frame by frame conversion. In some embodiments, the target regions can also be splice acceptor sites or splice donor sites, such that insertions or deletions during the repair process disrupt splicing and restore the huntingtin reading frame by splice site disruption and exon exclusion. In some embodiments, the target regions can also be aberrant stop codons, such that insertions or deletions during the repair process restore the huntingtin reading frame by eliminating or disrupting the stop codon. In some embodiments, any DNA abnormalities (for example, those listed above) occur within the poly-Q stretch of a human huntingtin gene, such that excision of the poly-Q stretch restores huntingtin functionality.

In certain embodiments, the presently disclosed DNA targeting a gRNA that may bind or target a polynucleotide within the huntingtin gene, or a truncation or a complement thereof. The gRNA molecule may comprise a polynucleotide corresponding to any one of SEQ ID NOs: 1-120, or a truncation or a complement thereof. In some embodiments, the target regions can be chosen, such that insertions or deletions during the repair process disrupt the huntingtin reading frame. In some embodiments, such disruptions result in ablation of huntingtin expression. In some embodiments, ablation of huntingtin expression is therapeutic to a subject with Huntington's Disease.

Disclosed herein is a genetic construct or a composition thereof for genome editing a target gene in a subject, such as, for example, a target gene in a neuron and/or an astrocyte of a subject. The genetic construct may be a vector. The vector may be a modified AAV vector. The composition may include a polynucleotide sequence encoding a CRISPR/Cas9-based gene editing system. The composition may deliver active forms of CRISPR/Cas9-based gene editing systems to the nervous system (e.g., a neuron). The presently disclosed genetic constructs can be used in correcting or reducing the effects of pathogenic expansions in the huntingtin gene involved in genetic diseases and/or other neurological conditions, such as, for example, HD. These compositions may further comprise a donor DNA or a transgene. These compositions may be used in genome editing, genome engineering, and correcting or reducing the effects of mutations in genes involved in genetic diseases and/or other neurological conditions.

A CRISPR/Cas9-based gene editing system specific for huntingtin gene editing is disclosed herein. The CRISPR/Cas9-based gene editing system may include Cas9 and at least one gRNA to target the huntingtin gene. The CRISPR/Cas9-based gene editing system may bind and recognize a target region. In some embodiments, the at least one gRNA molecule can bind and recognize a genomic target region. Target regions may include an intron of the huntingtin gene. Target regions may include an exon of the huntingtin gene.

The CRISPR/Cas9-based gene editing system composition may also include a viral delivery system. In certain embodiments, the vector is an adeno-associated virus (AAV) vector. The AAV vector is a small virus belonging to the genus Dependovirus of the Parvoviridae family that infects humans and some other primate species. AAV vectors may be used to deliver CRISPR/Cas9-based gene editing systems using various construct configurations. For example, AAV vectors may deliver Cas9 and gRNA expression cassettes on separate vectors or on the same vector. Alternatively, relatively smaller Cas9 proteins, such as those derived from Staphylococcus aureus (SaCas9) or Neisseria meningitidis (NmCas9) species, are used, then both the Cas9 and up to two gRNA expression cassettes may be combined in a single AAV vector within the 4.7 kb packaging limit.

In certain embodiments, the AAV vector is a recombinant AAV variant vector. The recombinant AAV variant vector may have enhanced cardiac and skeletal muscle tissue tropism. The recombinant AAV variant vector may be capable of delivering and expressing the CRISPR/Cas9-based gene editing system in the cell of a mammal. The recombinant AAV variant vector may deliver nucleases to cells of the nervous system (e.g., neurons, astrocytes, and/or glial cells) in vivo. The recombinant AAV variant vector may be based on one or more of several capsid types, including AAV1, AAV2, AAV5, AAV6, AAV8, AAV9, and AAVrh74. The recombinant AAV variant vector may be based on AAV2 pseudotype with alternative or engineered neurotropic AAV capsids that efficiently transduce neurons by systemic and local delivery (see, e.g., Srivastava, A. (2016). Current Opinion in Virology, 21, 75-80; Ghauri, M. S., & Ou, L. (2023). Biology, 12 (2), 186).

The compositions, as described above, may comprise one or more genetic constructs that encode the CRISPR/Cas9-based gene editing system, as disclosed herein. The genetic construct, such as a plasmid, may comprise a nucleic acid that encodes the CRISPR/Cas9-based gene editing system, such as the Cas9 protein and/or Cas9 fusion proteins and/or at least one of the gRNAs. The compositions, as described above, may comprise genetic constructs that encode an AAV vector and a nucleic acid sequence that encodes the CRISPR/Cas9-based gene editing system, as disclosed herein. The genetic construct, such as a plasmid, may comprise a nucleic acid that encodes the CRISPR/Cas9-based gene editing system. The compositions, as described above, may comprise genetic constructs that encode the modified lentiviral vector, as disclosed herein.

The genetic construct, such as a recombinant plasmid or recombinant viral particle, may comprise a nucleic acid that encodes the Cas9-fusion protein and at least one gRNA. In some embodiments, the genetic construct may comprise a nucleic acid that encodes the Cas9-fusion protein and at least two different gRNAs. In some embodiments, the genetic construct may comprise a nucleic acid that encodes the Cas9-fusion protein and more than two different gRNAs. In some embodiments, the genetic construct may comprise a promoter that is operably linked to the nucleotide sequence encoding the at least one gRNA molecule and/or a Cas9 molecule. In some embodiments, the promoter is operably linked to the nucleotide sequence encoding a first gRNA molecule, a second gRNA molecule, and/or a Cas9 molecule. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant lentivirus, recombinant adenovirus, and recombinant adenovirus associated virus. The genetic construct may be part of the genetic material in attenuated live microorganisms or recombinant microbial vectors which live in cells. The genetic constructs may comprise regulatory elements for gene expression of the coding sequences of the nucleic acid. Non-limiting examples of regulatory elements include a promoter, an enhancer, an initiation codon, a stop codon, or a polyadenylation signal.

In certain embodiments, the genetic construct is a vector. The vector can be an adeno-associated virus (AAV) vector, which encodes at least one Cas9 molecule and at least one gRNA molecule; the vector is capable of expressing the at least one Cas9 molecule and the at least gRNA molecule, in the cell of a mammal. The vector can be a plasmid. The vectors can be used for in vivo gene therapy. The vector may be recombinant. The vector may comprise heterologous nucleic acid encoding the fusion protein, such as the Cas9 fusion protein or CRISPR/Cas9-based gene editing system. The vector may be a plasmid. The vector may be useful for transfecting cells with nucleic acid encoding the Cas9 fusion protein or CRISPR/Cas9-based gene editing system, wherein the transformed host cell is cultured and maintained under conditions wherein expression of the Cas9 fusion protein or the CRISPR/Cas9-based gene editing system takes place.

Coding sequences may be optimized for stability and high levels of expression. In some instances, codons are selected to reduce secondary structure formation of the RNA such as that formed due to intramolecular bonding.

The vector may comprise heterologous nucleic acid encoding the CRISPR/Cas9-based gene editing system and may further comprise an initiation codon, which may be upstream of the CRISPR/Cas9-based gene editing system coding sequence, and a stop codon, which may be downstream of the CRISPR/Cas9-based gene editing system coding sequence. The initiation and termination codon may be in frame with the CRISPR/Cas9-based gene editing system coding sequence. The vector may also comprise a promoter that is operably linked to the CRISPR/Cas9-based gene editing system coding sequence. The promoter that is operably linked to the CRISPR/Cas9-based gene editing system coding sequence may be a promoter from simian virus 40 (SV40), a mouse mammary tumor virus (MMTV) promoter, a human immunodeficiency virus (HIV) promoter suct1 as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, a U6 promoter, such as the human U6 promoter, or a Rous sarcoma virus (RSV) promoter. The promoter may also be a promoter from a human gene such as human ubiquitin C (hUbC), human actin, human myosin, human hemoglobin, human muscle creatine, or human metallothionein. The promoter may also be a tissue specific promoter, such as a CNS- or neuron-specific promoter, natural or synthetic. Examples of CNS-specific promoters include a human synapsin 1 (hSyn) promoter (described in US Patent Application Publication No. 2019/0390204, which is incorporated by reference herein in its entirety), a MeCP2 promoter (described in US Patent Application Publication No. 2022/0049272), and a GFAP promoter (described in US Patent Application Publication No. 2020/0010903). In some embodiments, the expression of the gRNA and/or Cas9 protein is driven by tRNAs.

Each of the polynucleotide sequences encoding the gRNA molecule and/or Cas9 molecule may each be operably linked to a promoter. The promoters that are operably linked to the gRNA molecule and/or Cas9 molecule may be the same promoter. The promoters that are operably linked to the gRNA molecule and/or Cas9 molecule may be different promoters. The promoter may be a constitutive promoter, an inducible promoter, a repressible promoter, or a regulatable promoter. The promoter may be a tissue-specific promoter. The tissue-specific promoter may be a neuron-specific promoter. Non-limiting examples of neuron-specific promoters may include a hSyn promoter and a MeCP2 promoter.

The vector may also comprise a polyadenylation signal, which may be downstream of the CRISPR/Cas9-based gene editing system. The polyadenylation signal may be a SV40 polyadenylation signal, LTR polyadenylation signal, bovine growth hormone (bGH) polyadenylation signal, human growth hormone (HGH) polyadenylation signal, or human β-globin polyadenylation signal.

The vector may also comprise an enhancer upstream of the CRISPR/Cas9-based gene editing system, i.e., the Cas9 protein or Cas9 fusion protein coding sequence or sgRNAs, or the CRISPR/Cas9-based gene editing system. The enhancer may be necessary for DNA expression. The enhancer may be human actin, human myosin, human hemoglobin, human muscle creatine or a viral enhancer such as one from CMV, RSV or EBV. Polynucleotide functional polynucleotide enhancers are described in U.S. Pat. Nos. 5,593,972; 5,962,428; and WO/94/016737, the contents of each are hereby fully incorporated by reference for all purposes. The vector may also comprise a mammalian origin of replication in order to maintain the vector extrachromosomally and produce multiple copies of the vector in a cell. The vector may also comprise a regulatory sequence, which may be well suited for gene expression in a mammalian or human cell into which the vector is administered. The vector may also comprise a reporter gene, such as green fluorescent protein (“GFP”) and/or a selectable marker, such as hygromycin (“Hygro”).

The vector may be expression vectors or systems to produce protein by routine techniques and readily available starting materials including Sambrook et al., Molecular Cloning and Laboratory Manual, Second Ed., Cold Spring Harbor (1989), which is hereby incorporated fully by reference for all purposes. In some embodiments, the vector may comprise the nucleic acid sequence encoding the CRISPR/Cas9-based gene editing system, including the nucleic acid sequence encoding the Cas9 protein or Cas9 fusion protein and the nucleic acid sequence encoding the at least one gRNA.

The presently disclosed subject matter provides for compositions comprising the above-described genetic constructs. The pharmaceutical compositions as detailed herein can be formulated according to the mode of administration to be used. In some embodiments, the pharmaceutical compositions are injectable. In some embodiments, said injectable pharmaceutical compositions are sterile, pyrogen free and particulate free. In some embodiments, an isotonic formulation is preferably used. Generally, additives for isotonicity may include sodium chloride, dextrose, mannitol, sorbitol and lactose. In some embodiments, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS), Freumis incomplete adjuvant, LPS analog including monophosptloryl lipid A, muramyl peptides, quinone analogs, vesicles suct1 as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the composition may also include a transfection facilitating agent such as lipids, liposomes, including lecithin liposomes or other liposomes known in the art, as a DNA-liposome mixture (see for example International Patent Publication No. WO9324640), calcium ions, viral proteins, polyanions, polycations, nanoparticles, lipid nanoparticles or other known transfection facilitating agents. Preferably, the transfection facilitating agent is a polyanion, polycation, including poly-L-glutamate (LGS), or lipid.

Provided further herein is a kit, which may be used to correct a mutated huntingtin gene. The kit comprises at least a gRNA for correcting a mutated huntingtin gene and instructions for using the CRISPR/Cas9-based gene editing system. Also provided herein is a kit, which may be used for genome editing of a huntingtin gene in neurons, astrocytes and/or glial cells. The kit may comprise genetic constructs (e.g., vectors) or a composition comprising thereof for genome editing in one or more nervous system cell type, as described above, and instructions for using said composition.

Instructions included in kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips, and flash memory), optical media (e.g., CD ROM), and the like. As used herein, the term “instructions” may include the address of an internet site that provides the instructions.

The genetic constructs (e.g., vectors) or a composition comprising thereof for correcting a mutated huntingtin gene or genome editing of a huntingtin gene in one or more nervous system cells (e.g., neurons, astrocytes and/or glial cells) may include a modified AAV vector that includes a gRNA molecule(s) and a Cas9 molecule, as described above, that specifically binds and cleaves a region of the huntingtin gene. The CRISPR/Cas9-based gene editing system, as described above, may be included in the kit to specifically bind to and target a particular region in the mutated huntingtin gene. The kit may further include donor DNA, a different gRNA, or a transgene, as described above.

### Delivery and Administration Routes

Provided herein is a method for delivering the presently disclosed genetic construct (e.g., a vector) or a composition thereof to a cell. Upon delivery of the presently disclosed genetic construct or composition to the tissue, and thereupon the vector into the cells of the mammal, the transfected cells will express the gRNA molecule(s) and the Cas9 molecule. The genetic construct or composition may be administered to a mammal to alter gene expression or to re-engineer or alter the genome. For example, the genetic construct or composition may be administered to a mammal to correct the huntingtin gene in a mammal. The mammal may be human, non-human primate, cow, pig, sheep, goat, antelope, bison, water buffalo, bovids, deer, hedgehogs, elephants, llama, alpaca, mice, rats, or chicken, and preferably human, cow, pig, or chicken.

In some embodiments, the delivery of the gRNA molecule(s) and the Cas9 molecule is transfection of said composition as a nucleic acid molecule that is introduced into one or more cells following delivery to the surface of said cell. Transfections may include a transfection reagent, such as Lipofectamine 2000. In some embodiments, the delivery of the gRNA molecule(s) and the Cas9 molecule is electroporation of one or more cells. In some embodiments, the electroporation is performed using BioRad Gene Pulser Xcell or Amaxa Nucleofector devices. Several different buffers may be used, including BioRad electroporation solution, phosphate-buffered saline (PBS), Invitrogen OptiMEM, or Amaxa Nucleofector solution V (N.V.). In certain embodiments, a presently disclosed genetic construct (e.g., a vector) or a composition comprising at least one gRNA molecule and a Cas9 molecule is introduced into a HD patient cell. In certain embodiments, the genetic construct (e.g., a vector) or composition is introduced into a fibroblast cell from a HD patient, and the genetically corrected fibroblast cell can be treated to induce differentiation into central nervous system cells, which can be implanted into subjects to treat the subject. The genetically corrected cells can also be stem cells, such as induced pluripotent stem cells from HD patients or other similar progenitor cells. For example, the CRISPR/Cas9-based gene editing system may cause neuronal differentiation of an induced pluripotent stem cell.

In some embodiments, the genetic construct (e.g., a vector) encoding the gRNA molecule(s) and the Cas9 molecule is delivered to the mammal, optionally with any one of in vivo electroporation, liposome-mediated administration, nanoparticle-facilitated administration, and/or recombinant vectors. In some embodiments, said delivery to a subject is an injection selected from various routes including, but not limited to, intrapleural, intravenous, intraarterial, intraperitoneal, subcutaneous, intramuscular, intranasal intrathecal, intraarticular, intracranial, or a combination thereof. In some embodiments, said delivery is selected from any one of oral, parenteral, sublingual, transdermal, rectal, transmucosal, topical, via inhalation, via buccal administration, or combinations thereof. In certain embodiments, the presently disclosed genetic construct (e.g., a vector) or a composition is administered to a subject (e.g., a subject suffering from HD) intracranially, intravenously or a combination thereof. For veterinary use, the presently disclosed genetic constructs (e.g., vectors) or compositions may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, (e.g., microprojectile bombardment gene guns), or other physical methods such as electroporation, “hydrodynamic method”, or ultrasound. The recombinant vector can be delivered by any suitable viral genera. In some embodiments, the viral genera is selected from a recombinant lentivirus, a recombinant adenovirus, and/or a recombinant adeno-associated virus.

The presently disclosed genetic constructs (e.g., a vector) or a composition may be delivered to any mammal by several technologies including DNA injection (also referred to as DNA vaccination), optionally with in vivo electroporation, liposome-mediated, nanoparticle-facilitated, recombinant vectors such as recombinant lentivirus, recombinant adenovirus, and recombinant adenovirus associated virus. In some embodiments, the composition may be injected into the skeletal muscle to target motor neurons. For example, the composition may be injected into the tibialis anterior (TA) muscle or tail. In some embodiments, the composition may be injected into the cranial cavity of a subject. In some embodiments, the composition may be injected intravenously or intraarterially. In some embodiments, the presently disclosed genetic construct (e.g., a vector) or a composition thereof is administered by 1) tail vein injections (systemic) into adult mice; 2) intramuscular injections, for example, local injection into a muscle, such as the TA or gastrocnemius in adult mice; 3) intraperitoneal injections into P2 mice; or 4) facial vein injection (systemic) into P2 mice.

Any of these delivery methods and/or routes of administration can be utilized with a myriad of cell types. Cell types may include, but are not limited to, primary neurons, astrocytes, oligodendrocytes, Schwann cells, or glial cells, primary HD fibroblasts, induced pluripotent stem cells, neuronal progenitors, and hepatocytes. It is contemplated that cells may be modified ex vivo to isolate and expand clonal populations that include a genetically corrected huntingtin gene and are free of other nuclease-introduced mutations in protein coding regions of the genome. Alternatively, transient in vivo delivery of CRISPR/Cas9-based systems by non-viral or non-integrating viral gene transfer, or by direct delivery of purified proteins and gRNAs (collectively ribonucleoproteins or RNPs), optionally containing cell-penetrating motifs, may enable highly specific correction in situ with minimal or no risk of exogenous DNA integration.

### EXAMPLES

Suitable modifications and adaptations of the compositions and methods of the present disclosure, as described herein, are readily apparent, appreciable, and applicable to one of skill in the art. Such modifications and/or adaptations may be made using suitable equivalents without departing from the scope of the present disclosure or the aspects and embodiments disclosed herein.

Having now described the present disclosure in detail, the same will be more clearly understood by reference to the following examples, which are merely intended to illustrate some aspects and embodiments of the disclosure without limiting, in any way, the scope of the disclosure. The disclosures of all journal references, U.S. patents, and publications referred to herein are hereby incorporated by reference in their entireties for all purposes. The present disclosure details multiple embodiments and aspects, illustrated by the following non-limiting examples.

### Example 1—Designing and Producing a High-Throughput (HTP) Screening Library for gRNAs Targeting the HTT Gene

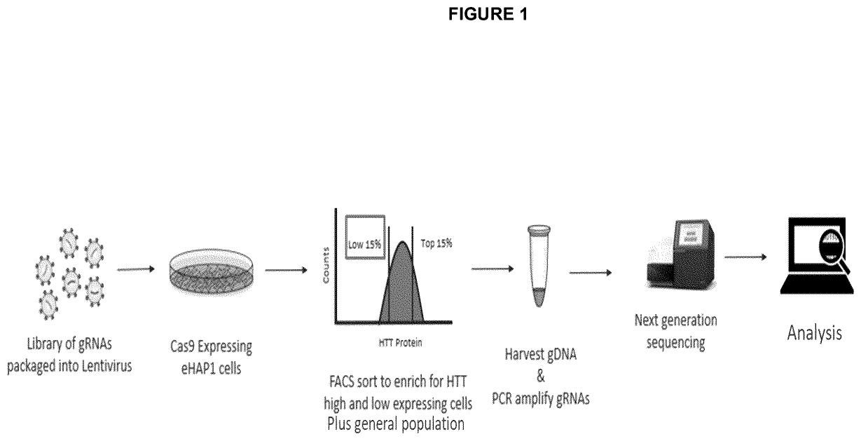

In order to identify gRNAs best suited to treat Huntington's Disease, a library of gRNAs capable of silencing HTT gene expression was assembled. The gRNAs were designed to be compatible with any one of SpCas9, SaCas9, or KKH-SaCas9 nucleases and to target any one of the 67 exons or the 5 kilobase region upstream of the ATG start codon in the human HTT gene, resulting in up to 1999 gRNAs from all 3 Cas9 variants that could potentially induce a knockout (KO) of HTT expression. Of these 1999 gRNAs, 1793 gRNAs were included in the high-throughput screen (schematized in FIG. 1) based upon computational predictions on specificity and reduction of off-target editing. These 1793 gRNAs were then cloned into their respective gRNA expression cassette in a lentivirus vector backbone before being packaged into lentivirus particles.

Three separate gRNA libraries (one for each Cas9 variant) were cloned via the Gibson Assembly technique using NEBuilder HiFi DNA Assembly Cloning Kit (New England Biolabs, NEB). Oligonucleotide inserts containing the gRNA target sequences were synthesized by Twist Biosciences, PCR amplified, and cloned into lentivirus vector backbones containing the corresponding gRNA scaffold. For instance, SpCas9 gRNAs were cloned into a lentivirus backbone containing the SpCas9 gRNA scaffold (SEQ ID NO: 243). KKH-SaCas9 gRNAs and SaCas9 gRNAs were cloned into a lentivirus backbone containing the SaCas9 gRNA scaffold (SEQ ID NO: 242). Cloned plasmids were transformed into electroporation-competent Endura E. coli (Lucigen), grown overnight, and purified via endotoxin-free maxiprep. Next-generation sequencing (NGS) on an Illumina MiSeq platform was performed to confirm representation of each gRNA in each of the 3 gRNA libraries before lentivirus production.

Three pools of lentivirus particles were harvested (one for each Cas9 variant gRNA library). 293 FT cells were transfected with the lentivirus plasmid library containing the gRNAs and lentivirus packaging plasmids (psPax2 and pMD2.G). 40 mM caffeine was added to stimulate lentivirus production within 24 hours of transfection. At 48 hours post transfection, lentivirus particles were harvested, filtered through a 0.44 μm PES filter, aliquoted, and frozen at −80° C. A functional titer was performed in eHAP1 cells, the cell type used for the HTP screen, using CellTiter Glo (Promega) to determine the volume of lentivirus needed to achieve 0.3 multiplicity of infection (MOI).

eHAP1 cells stably expressing SpCas9, SaCas9, or KKH SaCas9 were transduced with the corresponding lentivirus gRNA library at 0.3 MOI in the presence of 8 μg/ml polybrene. Media was changed 24 hours post transduction. 3-4 days post transduction, cells were selected with puromycin, which continued for 7 days until fluorescent-assisted cell sorting (FACS) screening.

### Example 2—HTP Screening Via FACS for gRNAs Targeting the HTT Gene

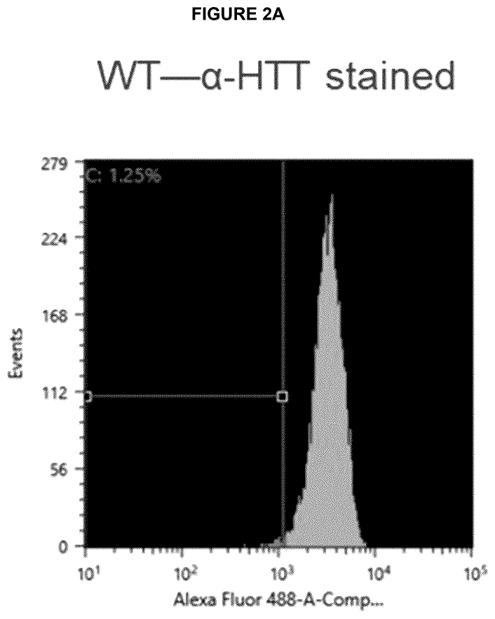

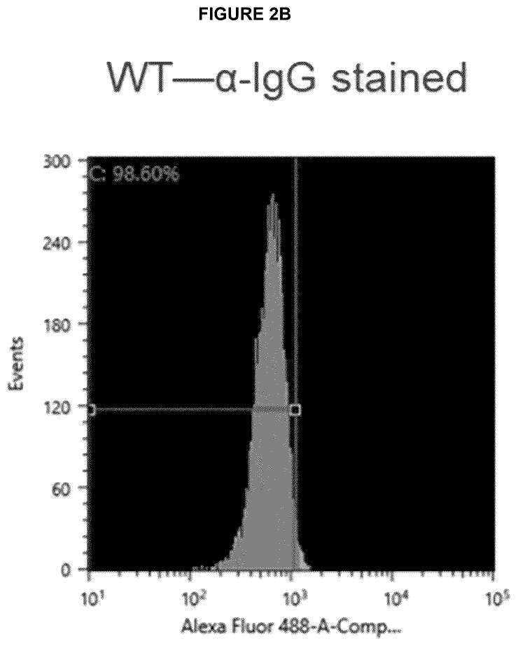

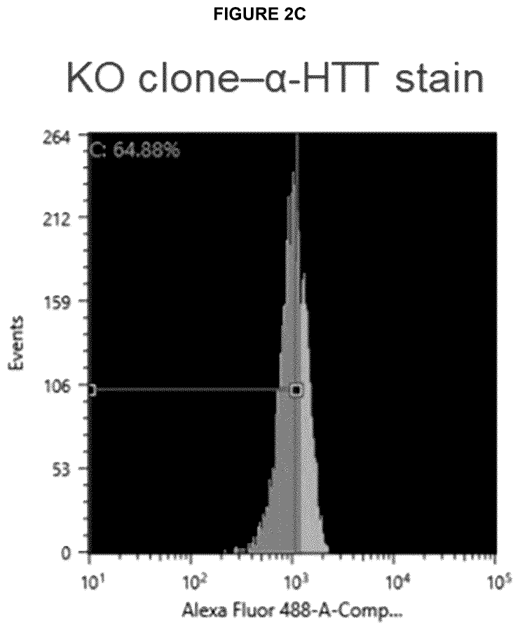

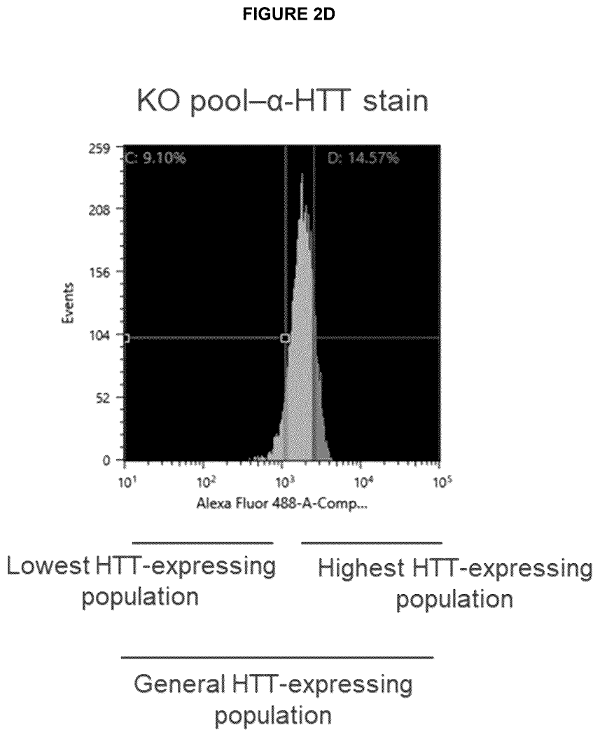

Cas9-expressing eHAP1 cells transduced with gRNA lentivirus and selected with puromycin were collected in Zn buffer to preserve nucleic acid and protein integrity before proceeding with FACS protocol. Briefly, cells preserved in Zn buffer were counted and adjusted to 2×106 cells/100 μL before being stained with either anti-HTT antibody (clone EP867Y) conjugated to Alexa Fluor 488 at 1:250 v/v dilution for 30 minutes in the dark, or the anti-IgG antibody conjugated to Alexa Fluor 488 as negative control. Wildtype (WT), unedited cells stained with anti-HTT antibody were used as positive control (FIGS. 2A, 2B), and a total HTT KO eHAP1 clone (clone 38) was used as negative control (FIG. 2C). After staining, cells were washed 3 times to remove unbound antibodies. An aliquot was taken to be the ‘General (Gen)’ population used to test for gRNA drop-out before FACS. Standard gating methods were used to remove debris and doublets from analysis, and a gate that captured the bottom or upper 10-15% of HTT-expressing cells were collected as the ‘Low’ or ‘High’ populations, respectively (FIG. 2D).

Genomic DNA from the collected cells were extracted using the GentraPure kit (Qiagen). The gRNA region was amplified via PCR using primers that already contained the necessary Illumina sequences needed for NGS. Amplified PCR products were purified, adjusted to equal concentration, and then sequenced on an Illumina MiSeq platform to assess the abundance of each gRNA from the 3 populations for each Cas9 variant.

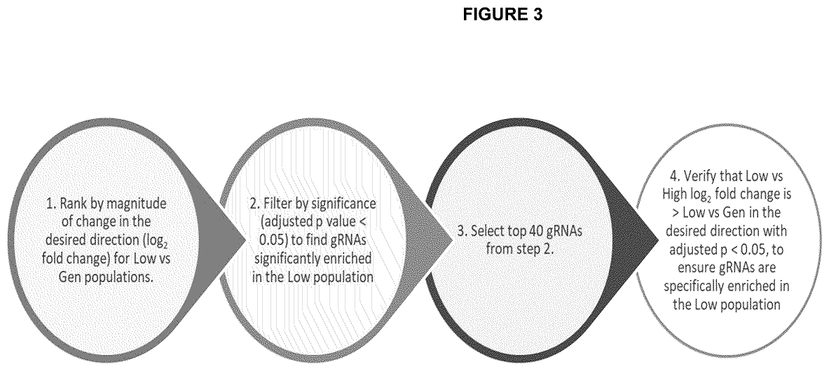

The CB² pipeline was used to analyze the sequencing results, and the gRNAs were ranked by magnitude of change in the desired direction (log₂ fold change) to order gRNAs that were enriched in the Low vs Gen populations (schematized in FIG. 3). Then, results were filtered by statistical significance using adjusted p-value<0.05 as a cut-off to select the top 40 gRNAs from each Cas9 variant gRNA library that were significantly enriched in the Low population. Lastly, the log₂ fold change comparing Low vs High (adjusted p-value<0.05) was used to verify that the selected gRNAs were specifically and significantly enriched in only the Low population and depleted from the High population.

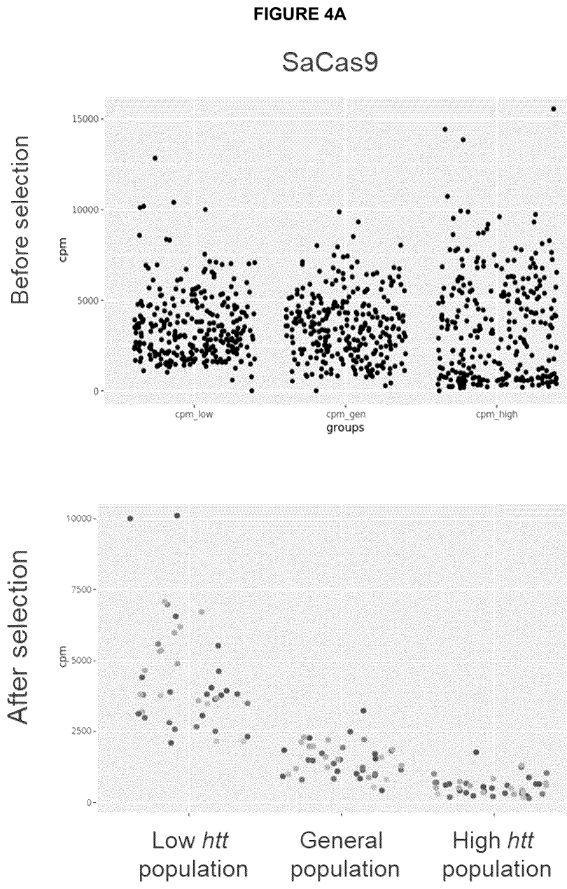

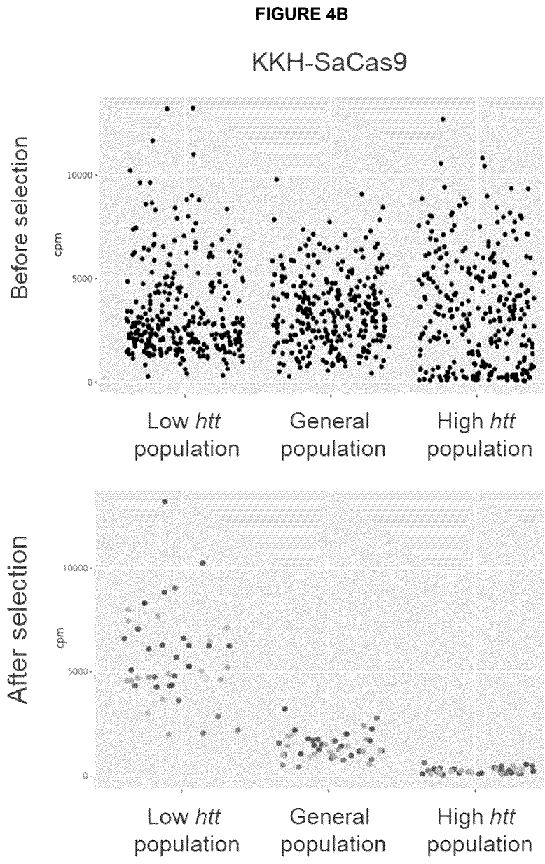

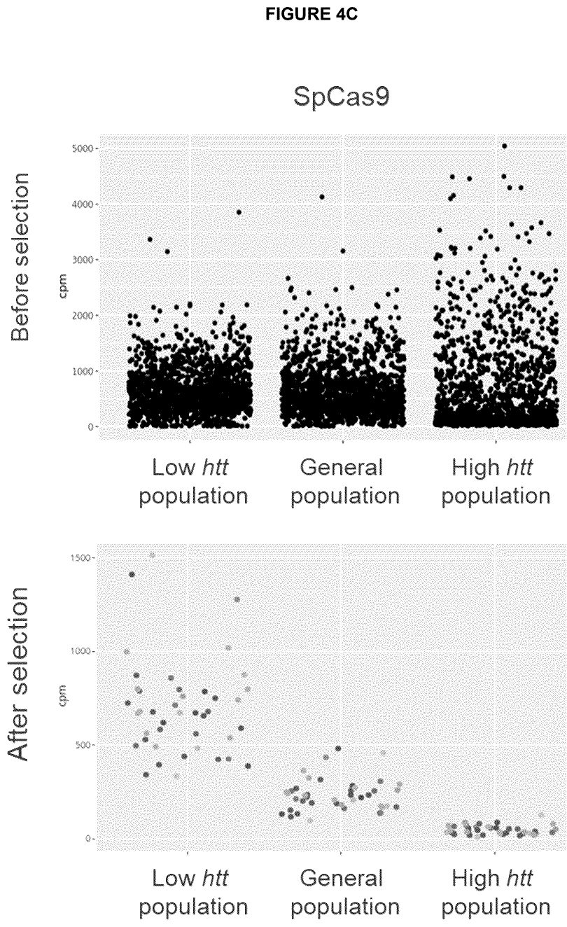

For each Cas9 variant library filtering resulted in identification of gRNAs that were significantly enriched in the low-HTT expressing population and absent from the high-HTT expressing population (FIGS. 4A-4C). Additionally, analysis of the general population found that gRNA coverage was 100%, i.e., no gRNA from the library was not represented in the analysis.

### Example 3—Validation of HTP Screening gRNA Hits

To validate the selected top 40 gRNAs from each Cas9 variant screen (SEQ ID NOs: 1-120; Tables 1-3), eHAP-1 cells expressing the appropriate Cas9 variant were transfected with each of the selected gRNAs separately prior to western blot analysis. Briefly, cell lysate was collected in RIPA buffer with protease inhibitors. BCA was used to determine the protein concentration in samples. The lysates were then subjected to SDS-PAGE in a 3-8% tris-acetate gel at 40 μg total protein/lane, transferred onto a PVDF membrane using iBlot2, blocked with 5% skim-milk, and incubated overnight at 4° C. with gentle rocking with anti-HTT antibody (clone D7F7, 1:1000 dilution) and anti-NMIIa antibody (1:1000 dilution) as a loading control. Primary antibodies were then removed with 3 washes, and the membrane was incubated with HRP-conjugated secondary antibodies. Signals were detected via chemiluminescence. HTT levels were analyzed by normalizing to the NMIIa loading control signals. Normalized HTT signals were then compared with the normalized HTT level in the WT control to calculate fold change using iBright Analysis Software.

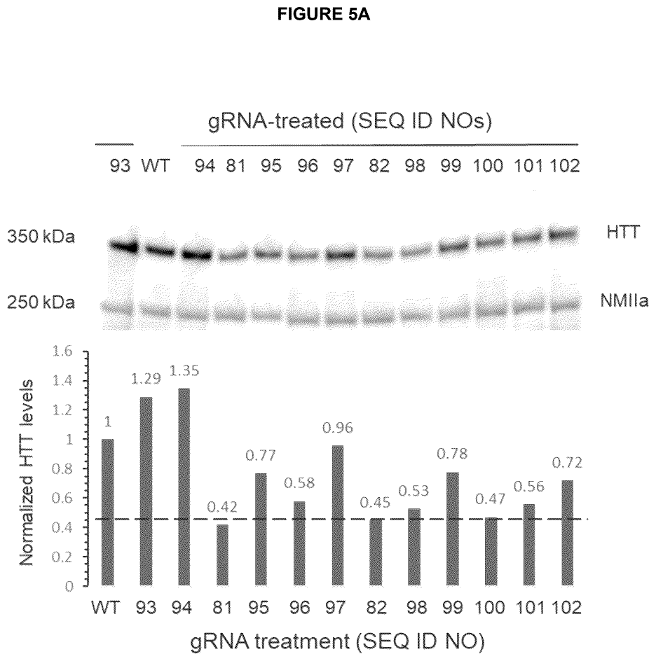

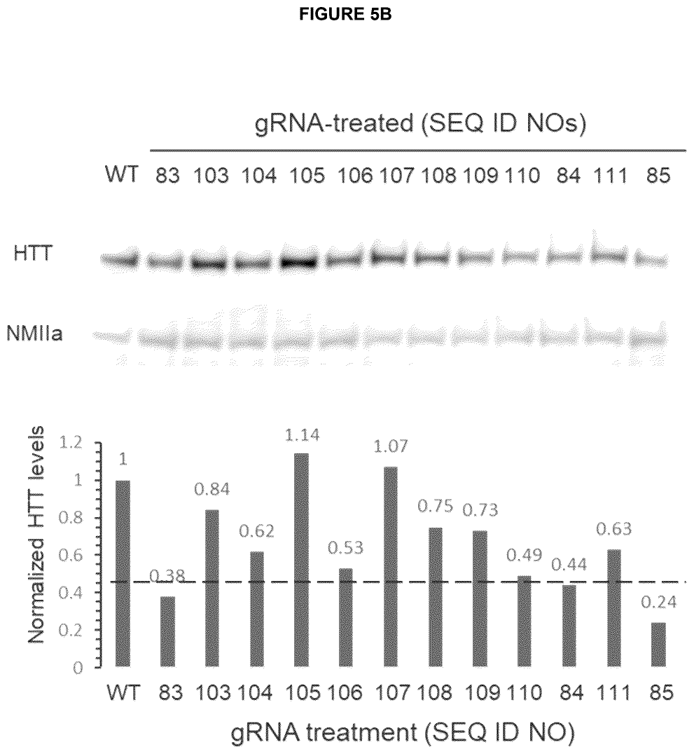

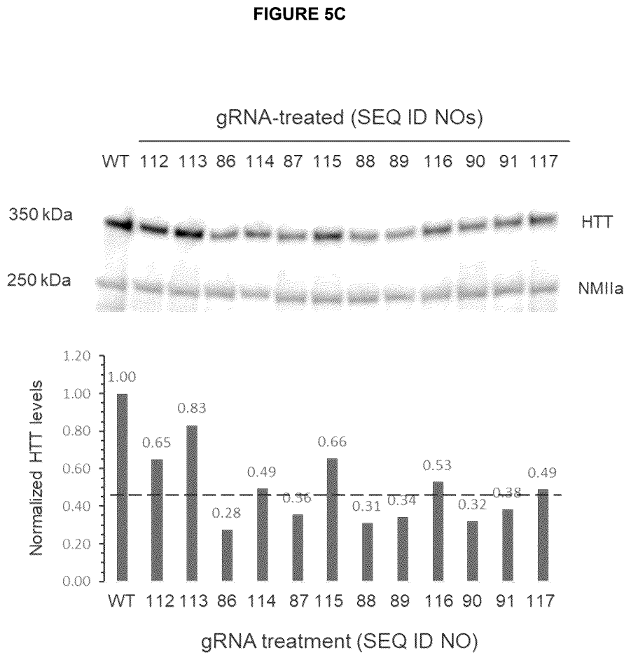

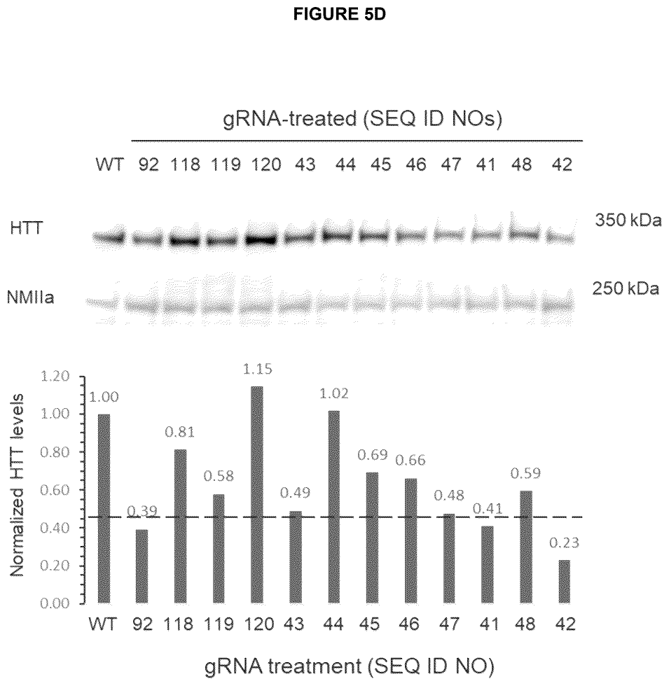

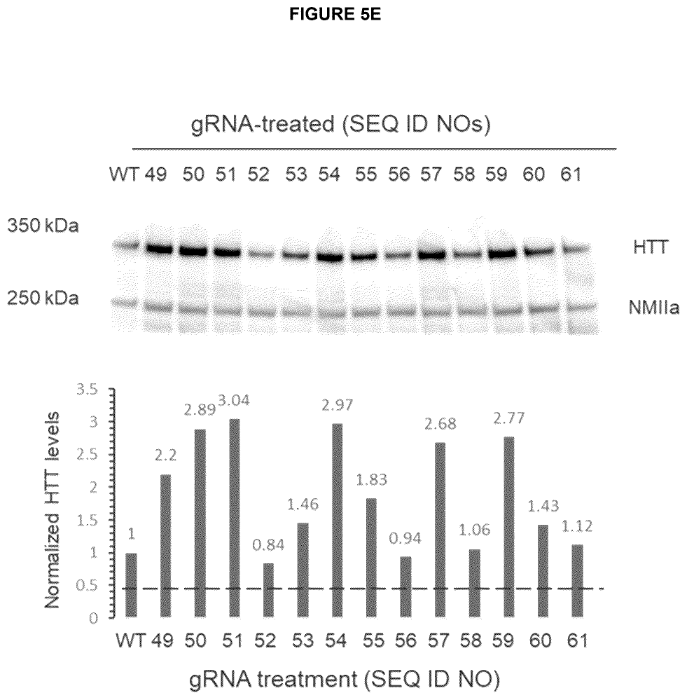

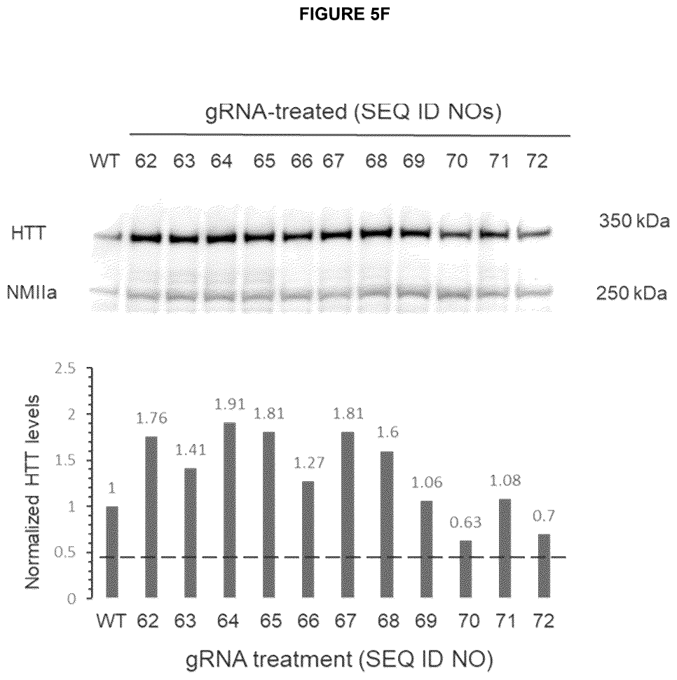

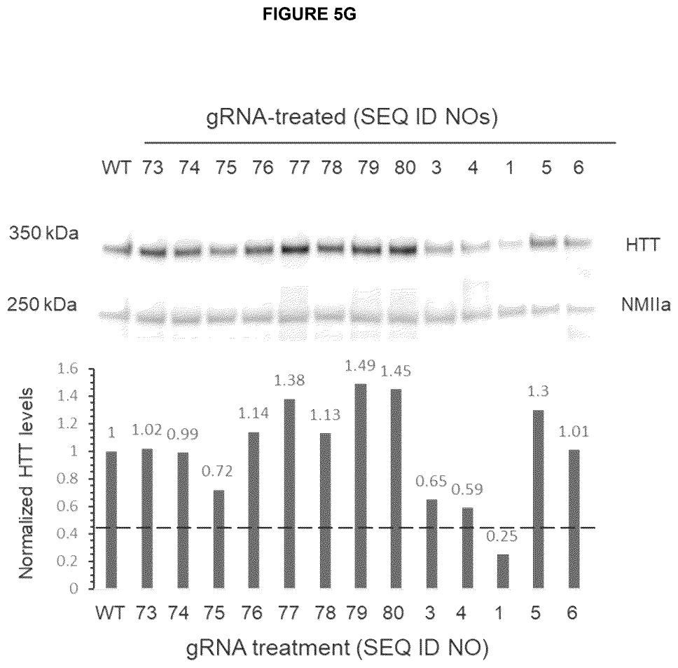

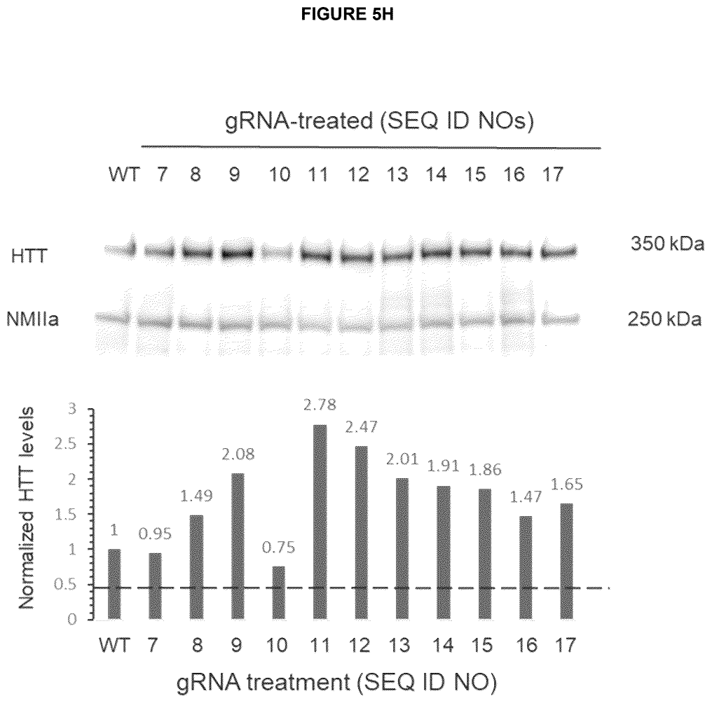

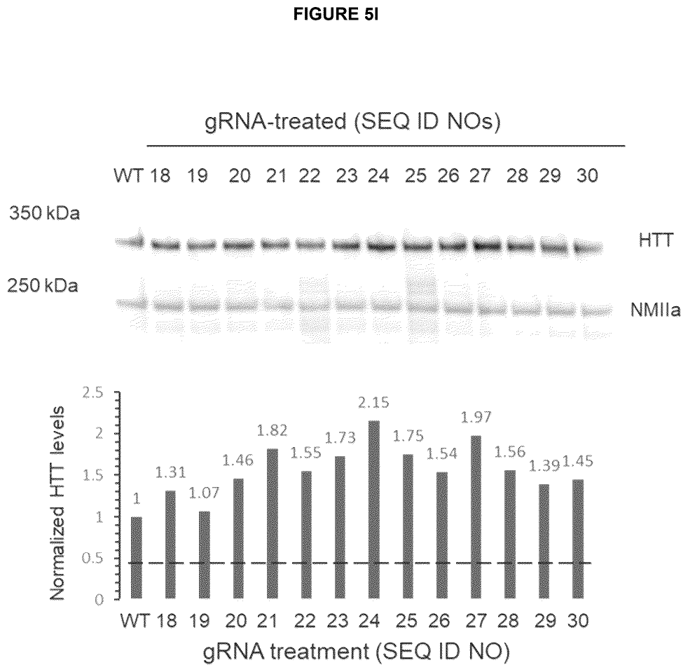

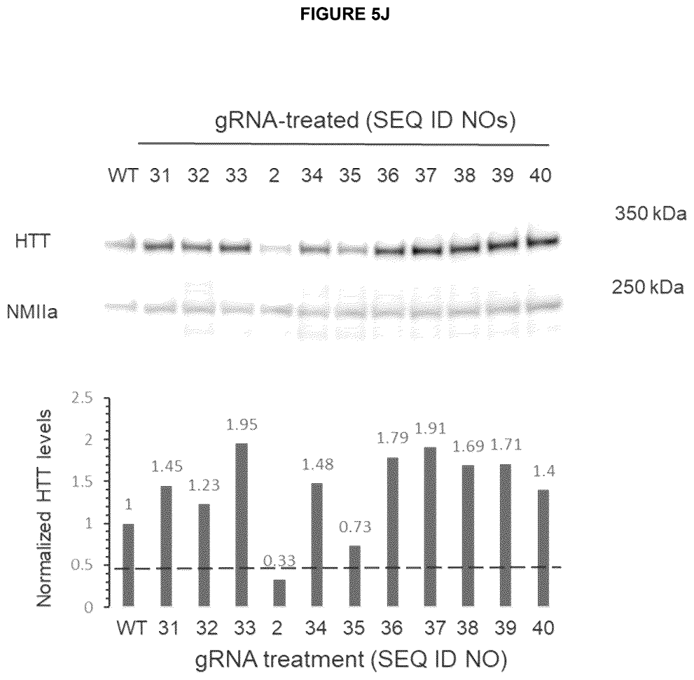

Initial western blot analysis found that the selected gRNAs exhibited variable ability to ablate detection of huntingtin protein; however, several gRNAs were validated to reduce huntingtin protein expression. Among the SpCas9 gRNAs, SEQ ID NOs: 81-92 were able to reduce huntingtin protein levels by at least 55% (FIGS. 5A-D). For KKH-SaCas9 gRNAs, SEQ ID NO: 41 and SEQ ID NO: 42 similarly reduced huntingtin expression below this cutoff (FIG. 5D). For SaCas9 gRNAs, SEQ ID NO: 1 and SEQ ID NO: 2 (FIGS. 5G-5J) were also capable of reducing huntingtin protein.

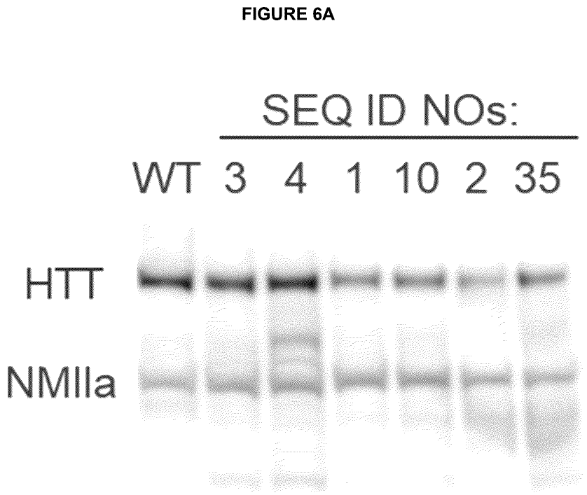

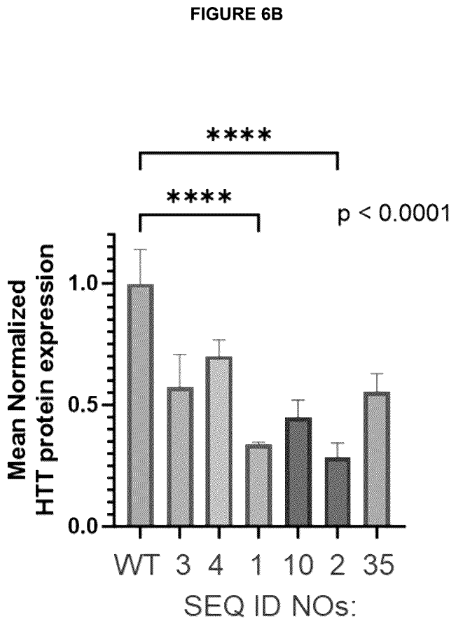

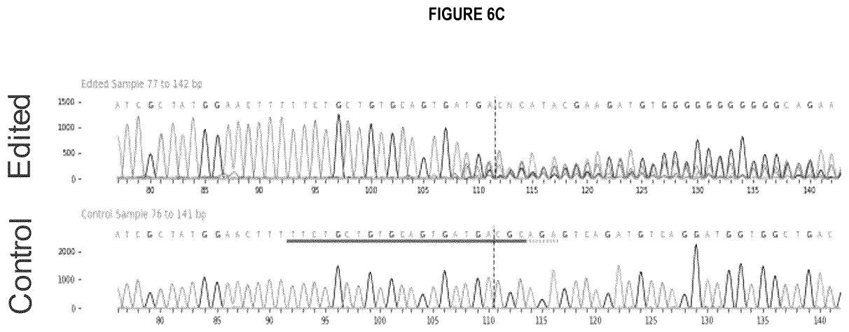

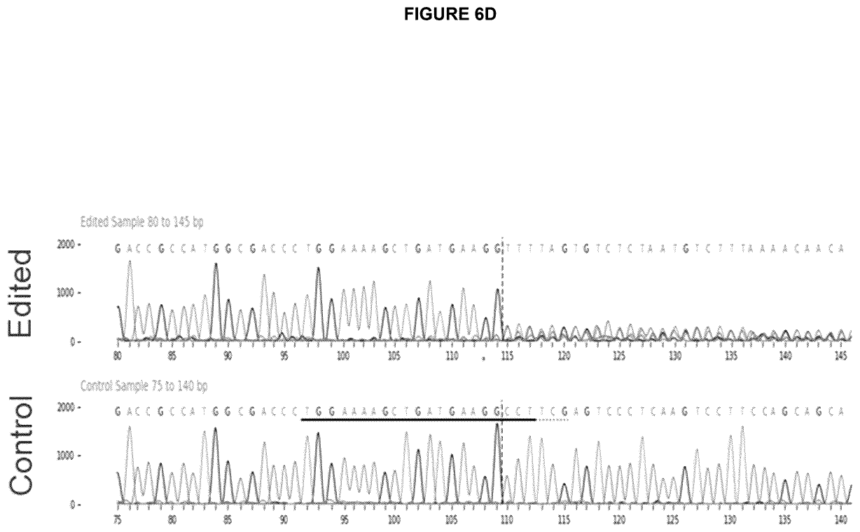

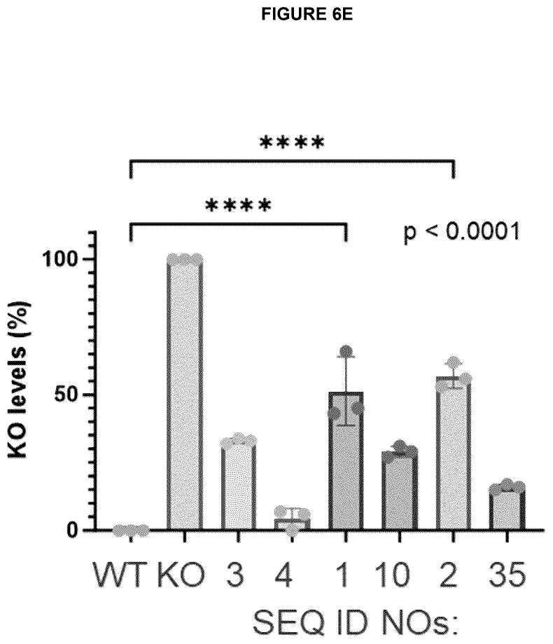

SEQ ID NO: 1 and SEQ ID NO: 2 were additionally validated by both confirmatory western blot and genomic sequencing. The confirmatory western blots (n=4) were performed as described previously and found that both SEQ ID NO: 1 and SEQ ID NO: 2 were able to facilitate a significant reduction in huntingtin protein as compared to both WT cell, while other unvalidated gRNAs did not (FIGS. 6A and 6B). To assess the level of gene editing at the endogenous HTT locus from the selected gRNAs, exons 1 and 3 of human HTT were amplified from extracted genomic DNA, purified, sequenced by Sanger sequencing, and analyzed using ICE (Synthego). Comparison of edited and WT cells showed that exon 1 was edited as predicted following introduction of SaCas9 and SEQ ID NO: 1 (FIG. 6C). Similarly, exon 3 was also edited as predicted following introduction of SaCas9 and SEQ ID NO: 2 (FIG. 6D). Indeed, only SEQ ID NO: 1 and SEQ ID NO: 2 exhibited significant knockout compared to WT cells, among several selected gRNAs (FIG. 6E). Taken together, these results validated the methodology of the library design and the specific activity of the selected gRNAs.

### Example 4—Vector Components for Gene Editing System Targeting the HTT Gene

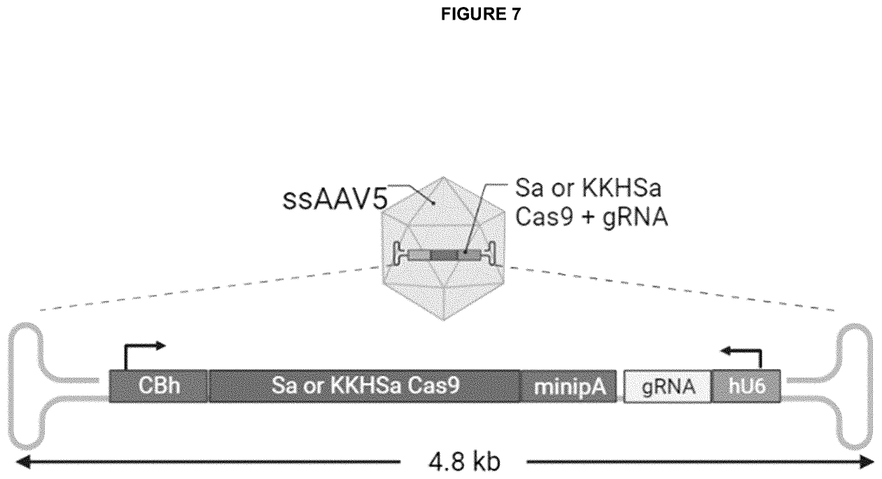

The single-polynucleotide CRISPR/Cas9 systems developed for the treatment of HD (schematized in FIG. 7) have all necessary editing components on a single vector, streamline vector production (single therapeutic agent) and negating the need to calculate ratios of multiple vectors. Exemplary sequences included in some or all of the vectors herein described (either dual-vector or single-polynucleotide approaches) are shown in Table 5.

### Example 5—Treatment of HD Symptoms In Vivo (Mouse Model)

This example details the assessment of gene editing systems to ameliorate symptoms in a murine model of Huntington's Disease and to demonstrate safety of this treatment as a therapeutic approach.

Hu97/18 mice are dosed once with AAV5 vectors containing SaCas9 CRISPR gene editing systems and a gRNA at 6.5×10¹⁰ vg/mouse (or 0.9% sterile saline control) via bilateral intrastriatal injection into the striatal regions of the brain according to the experimental design in Table 6. In the case of a dual-vector approach, where Cas9 and gRNA are on separate AAV5 vectors, the dose of Cas9:gRNA is delivered at a 1:3 ratio at 3.25×10¹⁰ vg/mouse: 9.75×10¹⁰ vg/mouse, respectively.

Following dosing, animals are observed for general health and well-being until approximately 2 or 15 months post injection (mpi) (or when mice reach 18 months of age). For simplicity, each month post injection/dosing is considered a calendar month. To allow flexibility, sample/data collection (e.g., body weight, blood, tissue) can occur within +7 days of the target date.

At the terminal timepoint of 2 mpi (for groups 1, 3, 5, 7,9-13, 15, 16, and 18 in Table 6), animals are euthanized, undergo blood collection, and a necropsy to dissect out fresh striatal and adjacent cortical brain regions. These brain tissues are then snap frozen and stored at −80° C.

At the terminal timepoint of 15 mpi (for groups 2, 4, 6, 8, 14, 17, and 19 in Table 1), animals are euthanized, undergo blood collection, except for mice that are transcardially perfused and fixed. Necropsies are conducted to extract various brain and non-brain tissues.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1
SaCas9-compatible gRNAs and associated PAMs targeting the HTT gene

| | Target | |
|---|---|---|
| Protospacer Sequence | exon | PAM Sequence |
| TTCTGCTGTGCAGTGATGACG (SEQ ID NO: 1) | 3 | CAGAGT (SEQ ID NO: 121) |
| TGGAAAAGCTGATGAAGGCCT (SEQ ID NO: 2) | 1 | TCGAGT (SEQ ID NO: 122) |
| TGCTGCGGAAAACACTGAAGG (SEQ ID NO: 3) | 18 | ATGAGT (SEQ ID NO: 123) |
| CACGGTGCCGCTCACACGGGC (SEQ ID NO: 4) | 48 | CAGAGT (SEQ ID NO: 124) |
| GTACTGTGCCAGGGCCCGGGC (SEQ ID NO: 5) | 49 | CAGAGT (SEQ ID NO: 125) |
| GGACACAATGGACAAGAGGTG (SEQ ID NO: 6) | 15 | CAGAGT (SEQ ID NO: 126) |
| GAGGAAGCTGAGCATTATCAG (SEQ ID NO: 7) | 56 | AGGGAT (SEQ ID NO: 127) |
| TGTCAGCGCGTCCACCAGCCC (SEQ ID NO: 8) | 66 | GTGGGT (SEQ ID NO: 128) |
| ATGGATCCCCTCCAAGCACTG (SEQ ID NO: 9) | 44 | AAGAGT (SEQ ID NO: 129) |
| TTTGCTTGGAGATGAAGACCC (SEQ ID NO: 10) | 21 | CAGGGT (SEQ ID NO: 130) |
| CCTCAGGGTGAGCAGCACGCC (SEQ ID NO: 11) | 8 | AAGAAT (SEQ ID NO: 131) |
| GGCACAGTGGTAAATGATGGA (SEQ ID NO: 12) | 63 | GGGGGT (SEQ ID NO: 132) |
| TGGGAACCAAGCTGACGAGAG (SEQ ID NO: 13) | 25 | CAGGGT (SEQ ID NO: 133) |
| CTCGAACAAGCAAGAGACCCG (SEQ ID NO: 14) | 6 | AAGAAT (SEQ ID NO: 134) |
| CATTGACTCTCATGAAGCCCT (SEQ ID NO: 15) | 38 | TGGAGT (SEQ ID NO: 135) |
| TGGAAGAACTCAACAGAATCC (SEQ ID NO: 16) | 45 | AGGAAT (SEQ ID NO: 136) |
| GGATGAACAGGCCGCTGGCAG (SEQ ID NO: 17) | 43 | CAGAGT (SEQ ID NO: 137) |
| TTGATCCATGAGCAGATCCCG (SEQ ID NO: 18) | 50 | CTGAGT (SEQ ID NO: 138) |
| ACAAGTAAATCCTCATCACTG (SEQ ID NO: 19) | 28 | GGGAGT (SEQ ID NO: 139) |
| GGGTGATGCTGTCTGGAAGTG (SEQ ID NO: 20) | 63 | AGGAGT (SEQ ID NO: 140) |
| ATACGGAGAGAAGGAGAGCTC (SEQ ID NO: 21) | 39 | CTGAAT (SEQ ID NO: 141) |
| CAGCATTCCCTGAGATCCCCG (SEQ ID NO: 22) | 53 | TGGAGT (SEQ ID NO: 142) |
| GCTGGTCAAGCTGAGTGTGGA (SEQ ID NO: 23) | 63 | CAGAGT (SEQ ID NO: 143) |
| GCTCCATGCTTAAGCCTAGGG (SEQ ID NO: 24) | 48 | ATGAGT (SEQ ID NO: 144) |
| GGCCTGCAGATTGGACAGCCC (SEQ ID NO: 25) | 13 | CAGGAT (SEQ ID NO: 145) |
| CACTATGGAAAATAACCTTTC (SEQ ID NO: 26) | 23 | AAGAGT (SEQ ID NO: 146) |
| TCCCATCATTCAGGTCCATGG (SEQ ID NO: 27) | 12 | CAGGGT (SEQ ID NO: 147) |
| GTCCTGCAGCAGTGCCACAAG (SEQ ID NO: 28) | 37 | GAGAAT (SEQ ID NO: 148) |
| GCACGCCAAGAATCAGCAGAG (SEQ ID NO: 29) | 8 | TGGAGT (SEQ ID NO: 149) |
| GTGAGATGGTGGCAGAAATGG (SEQ ID NO: 30) | 52 | TGGAGT (SEQ ID NO: 150) |
| AGAGACGAAACCTGTCCAGCA (SEQ ID NO: 31) | 46 | GGGAAT (SEQ ID NO: 151) |
| ACAGTAGATGAGGGAGCAGGC (SEQ ID NO: 32) | 50 | GTGGGT (SEQ ID NO: 152) |
| GAAAGGCTTTCCTTGTGAAGC (SEQ ID NO: 33) | 65 | CAGAGT (SEQ ID NO: 153) |
| GCATCACACCCCTGAGGGAGG (SEQ ID NO: 34) | 58 | AGGAAT (SEQ ID NO: 154) |
| CCCAATGCCCCCGACTGCGGT (SEQ ID NO: 35) | 9 | CAGGGT (SEQ ID NO: 155) |
| GACACCAGAGCAGCACCAGGG (SEQ ID NO: 36) | 41 | CCGGGT (SEQ ID NO: 156) |
| CCCCAGATGTCTGGAGAAGAG (SEQ ID NO: 37) | 42 | GAGGAT (SEQ ID NO: 157) |
| TGCCTCCACTGAGTGCCTCAG (SEQ ID NO: 38) | 25 | ATGAGT (SEQ ID NO: 158) |
| CTTTTCCTGGAGGAACTCCAC (SEQ ID NO: 39) | 53 | GGGGAT (SEQ ID NO: 159) |
| GAAGGCATTCATATCTTCAGC (SEQ ID NO: 40) | 47 | AGGAAT (SEQ ID NO: 160) |

TABLE 2
KKH-SaCas9-compatible gRNAs and associated PAMs targeting the HTT gene

| | Target | |
|---|---|---|
| Protospacer Sequence | exon | PAM Sequence |
| CACGGTGCCGCTCACACGGGC (SEQ ID NO: 41) | 48 | CAGAGT (SEQ ID NO: 161) |
| CTCGAACAAGCAAGAGACCCG (SEQ ID NO: 42) | 6 | AAGAAT (SEQ ID NO: 162) |
| TGCTGCGGAAAACACTGAAGG (SEQ ID NO: 43) | 18 | ATGAGT (SEQ ID NO: 163) |
| TGCTGCGGAAAACACTGAAGG (SEQ ID NO: 44) | 15 | ATGAGT (SEQ ID NO: 164) |
| CAGCATTCCCTGAGATCCCCG (SEQ ID NO: 45) | 53 | TGGAGT (SEQ ID NO: 165) |
| CCTCAGGGTGAGCAGCACGCC (SEQ ID NO: 46) | 8 | AAGAAT (SEQ ID NO: 166) |
| GAGGAAGCTGAGCATTATCAG (SEQ ID NO: 47) | 56 | AGGGAT (SEQ ID NO: 167) |
| ACTCCAAATGCAAACTGGGAA (SEQ ID NO: 48) | 24 | GGCAGT (SEQ ID NO: 168) |
| TTTGCTTGGAGATGAAGACCC (SEQ ID NO: 49) | 21 | CAGGGT (SEQ ID NO: 169) |
| TTGATCCATGAGCAGATCCCG (SEQ ID NO: 50) | 50 | CTGAGT (SEQ ID NO: 170) |
| GAAAGGCTTTCCTTGTGAAGC (SEQ ID NO: 51) | 65 | CAGAGT (SEQ ID NO: 171) |
| TTCTGCTGTGCAGTGATGACG (SEQ ID NO: 52) | 3 | CAGAGT (SEQ ID NO: 172) |
| ACAAGTAAATCCTCATCACTG (SEQ ID NO: 53) | 28 | GGGAAT (SEQ ID NO: 173) |
| GGATGAACAGGCCGCTGGCAG (SEQ ID NO: 54) | 43 | CAGAGT (SEQ ID NO: 174) |
| AGAGACGAAACCTGTCCAGCA (SEQ ID NO: 55) | 46 | GGGAAT (SEQ ID NO: 175) |
| CAGATGTCAGGATGGTGGCTG (SEQ ID NO: 56) | 3 | ACGAAT (SEQ ID NO: 176) |
| TGTCAGCGCGTCCACCAGCCC (SEQ ID NO: 57) | 66 | GTGGGT (SEQ ID NO: 177) |
| GCTCCATGCTTAAGCCTAGGG (SEQ ID NO: 58) | 48 | ATGAGT (SEQ ID NO: 178) |
| TGGGAACCAAGCTGACGAGAG (SEQ ID NO: 59) | 25 | CAGGGT (SEQ ID NO: 179) |
| TGGAAGAACTCAACAGAATCC (SEQ ID NO: 60) | 45 | AGGAAT (SEQ ID NO: 180) |
| TGTGACGCTGACAGAACTGCG (SEQ ID NO: 61) | 60 | AAGGGT (SEQ ID NO: 181) |
| CATTGACTCTCATGAAGCCCT (SEQ ID NO: 62) | 38 | TGGAGT (SEQ ID NO: 182) |
| GGGTGATGCTGTCTGGAAGTG (SEQ ID NO: 63) | 63 | AGGAGT (SEQ ID NO: 183) |
| GGATGGTCACCCAAACCGGGA (SEQ ID NO: 64) | 53 | GGGGAT (SEQ ID NO: 184) |
| GCAGTCGTCCATGGTCCGGGA (SEQ ID NO: 65) | 66 | CTGGGT (SEQ ID NO: 185) |
| GCTGGTCAAGCTGAGTGTGGA (SEQ ID NO: 66) | 63 | CAGAGT (SEQ ID NO: 186) |
| ATGGATCCCCTCCAAGCACTG (SEQ ID NO: 67) | 44 | AAGAGT (SEQ ID NO: 187) |
| GGCACAGTGGTAAATGATGGA (SEQ ID NO: 68) | 63 | GGGGGT (SEQ ID NO: 188) |
| GAATCTGACCTGGTCCAACAC (SEQ ID NO: 69) | 47 | TGGGAT (SEQ ID NO: 189) |
| GTCCTGCAGCAGTGCCACAAG (SEQ ID NO: 70) | 37 | GAGAAT (SEQ ID NO: 190) |
| TGCACGGCGTCCTCTATGTGC (SEQ ID NO: 71) | 61 | TGGAGT (SEQ ID NO: 191) |
| ATTCCGGCCCTACGTCCAGAG (SEQ ID NO: 72) | 62 | GATAGT (SEQ ID NO: 192) |
| GTACTGTGCCAGGGCCCGGGC (SEQ ID NO: 73) | 49 | CAGAGT (SEQ ID NO: 193) |
| CCCCAGATGTCTGGAGAAGAG (SEQ ID NO: 74) | 42 | GAGGAT (SEQ ID NO: 194) |
| GTGAGATGGTGGCAGAAATGG (SEQ ID NO: 75) | 52 | TGGAGT (SEQ ID NO: 195) |
| CATGCGAGCCAGCACACGGAA (SEQ ID NO: 76) | 44 | AGGGGT (SEQ ID NO: 196) |
| GAAGGCATTCATATCTTCAGC (SEQ ID NO: 77) | 47 | AGGAAT (SEQ ID NO: 197) |
| ACAGTAGATGAGGGAGCAGGC (SEQ ID NO: 78) | 50 | GTGGGT (SEQ ID NO: 198) |
| TCTGGAGCGTGGTCTCCTCCA (SEQ ID NO: 79) | 50 | CAGAGT (SEQ ID NO: 199) |
| AGTGAAATTTCTGGTGGCCAG (SEQ ID NO: 80) | 48 | AAGAGT (SEQ ID NO: 200) |

TABLE 3
SpCas9-compatible gRNAs and associated PAMs targeting the HTT gene

| | Target | |
|---|---|---|
| Protospacer Sequence | exon | PAM Sequence |
| CAAGACCACAATGTTGTGAC (SEQ ID NO: 81) | 9 | CGG (SEQ ID NO: 201) |
| CTTCCCAATGTCCTGCAGTG (SEQ ID NO: 82) | 29 | TGG (SEQ ID NO: 202) |
| AGAATCAGTCCAGGAGACCT (SEQ ID NO: 83) | 6 | TGG (SEQ ID NO: 203) |
| GGAAATCAGAACCCTCAAAA (SEQ ID NO: 84) | 39 | TGG (SEQ ID NO: 204) |
| CCAGAGGATAGTTCTCAATG (SEQ ID NO: 85) | 62 | AGG (SEQ ID NO: 205) |
| CAGGATCCTGGCCACCACTC (SEQ ID NO: 86) | 65 | TGG (SEQ ID NO: 206) |
| GTAGTAGCCGGAGACAGAGA (SEQ ID NO: 87) | 56 | AGG (SEQ ID NO: 207) |
| TTAAGTGCTCGGAGTCATGG (SEQ ID NO: 88) | 43 | AGG (SEQ ID NO: 208) |
| TTCCATGATCACCACCCACC (SEQ ID NO: 89) | 41 | CGG (SEQ ID NO: 209) |
| ATTGTGTCCTTCTTGAGTGC (SEQ ID NO: 90) | 7 | TGG (SEQ ID NO: 210) |
| GCACAGTGGTAAATGATGGA (SEQ ID NO: 91) | 63 | GGG (SEQ ID NO: 211) |
| GATGAGGGTCCCACAGAGAA (SEQ ID NO: 92) | 17 | TGG (SEQ ID NO: 212) |
| TCATCGATGTGCTGACTCTG (SEQ ID NO: 93) | 19 | AGG (SEQ ID NO: 213) |
| GTTTGCTGAAGAAAGATTCC (SEQ ID NO: 94) | 16 | GGG (SEQ ID NO: 214) |
| TAATGTATTTAACACTCCAA (SEQ ID NO: 95) | 38 | GGG (SEQ ID NO: 215) |
| ACTTTCTATTGCCAGGAACT (SEQ ID NO: 96) | 40 | AGG (SEQ ID NO: 216) |
| CAATCTGCAGGCCCAAATAC (SEQ ID NO: 97) | 13 | TGG (SEQ ID NO: 217) |
| CATCGATCATGGAGACCCAC (SEQ ID NO: 98) | 17 | AGG (SEQ ID NO: 218) |
| ATGTGTGCAGTTACAGAAGC (SEQ ID NO: 99) | 33 | AGG (SEQ ID NO: 219) |
| AGACGACTTCTTCCCACCCC (SEQ ID NO: 100) | 65 | AGG (SEQ ID NO: 220) |
| GAGCATTATCAGAGGGATTG (SEQ ID NO: 101) | 56 | TGG (SEQ ID NO: 221) |
| CAGCTCAGCAAACCTCCACA (SEQ ID NO: 102) | 5 | GGG (SEQ ID NO: 222) |
| GGTACCGACAACCAGTATTT (SEQ ID NO: 103) | 13 | GGG (SEQ ID NO: 223) |
| GGGCTCCGGTCACAACATTG (SEQ ID NO: 104) | 9 | TGG (SEQ ID NO: 224) |
| GGAGATTAAATACGGAGAGA (SEQ ID NO: 105) | 39 | AGG (SEQ ID NO: 225) |
| ATCACTGCGGAACAGCCTAG (SEQ ID NO: 106) | 41 | TGG (SEQ ID NO: 226) |
| TCTCTTGCCACGGCCACTAC (SEQ ID NO: 107) | 22 | TGG (SEQ ID NO: 227) |
| GAGGAACAGTTCCTATTGGC (SEQ ID NO: 108) | 19 | TGG (SEQ ID NO: 228) |
| GTCCTGTACTGGAGGCTCGT (SEQ ID NO: 109) | 43 | GGG (SEQ ID NO: 229) |
| ATAATGTATTTAACACTCCA (SEQ ID NO: 110) | 38 | AGG (SEQ ID NO: 230) |
| ATGCGAGCCAGCACACGGAA (SEQ ID NO: 111) | 44 | AGG (SEQ ID NO: 231) |
| GACATCATGAACAAAGTCAT (SEQ ID NO: 112) | 65 | CGG (SEQ ID NO: 232) |
| GTTACAGCTCGAGCTCTATA (SEQ ID NO: 113) | 4 | AGG (SEQ ID NO: 233) |
| GCTGCACGGTGCCGCTCACA (SEQ ID NO: 114) | 48 | CGG (SEQ ID NO: 234) |
| AAATGTTAATCACCTTCAGC (SEQ ID NO: 115) | 26 | AGG (SEQ ID NO: 235) |
| TCAGGACATCATGCAGTTTG (SEQ ID NO: 116) | 28 | AGG (SEQ ID NO: 236) |
| GATCACCACCCACCCGGCCC (SEQ ID NO: 117) | 41 | TGG (SEQ ID NO: 237) |
| TAGAGAAATAGTACGAAGAG (SEQ ID NO: 118) | 42 | GGG (SEQ ID NO: 238) |
| AGTTTGCTGAAGAAAGATTC (SEQ ID NO: 119) | 16 | CGG (SEQ ID NO: 239) |
| GGAAGAAGTCGTCTAGAAAC (SEQ ID NO: 120) | 65 | TGG (SEQ ID NO: 240) |

TABLE 4
Exemplary CRISPR nucleases and minimum requirements for gene editing
function.

| Nuclease (bacterial | Size | PAM | Nucleic acid target | Protospacer |
|---|---|---|---|---|
| species of origin) | (aa) | (5′→3′) | (end type generated) | length (nt) |
| Cas9 (S. pyogenes) | 1368 | NGG | dsDNA (blunt) | 20 |
| Cas9 (S. aureus) | 1053 | NNGRRT | dsDNA (blunt) | 21 |
| KKH-SaCas9 (S. aureus) | 1053 | NPGRRT | dsDNA (blunt) | 21 |
| Cas9 (S. thermophilus) | 1122 | NNAGAAW | dsDNA (blunt) | 20 |
| Cas9 (N. meningitidis) | 1083 | NNNNGATT | dsDNA (blunt) | 24 |
| Cpf1 (F. novicida) | 1300 | (T)TTV | SSDNA/dsDNA | 23-25 |
| | | | (staggered) | |
| Cpf1 (Lachnospiraceae sp.) | 1228 | TTTV | SSDNA/dsDNA | 23-25 |
| | | | (staggered) | |
| Cpf1 (Acidaminococcus sp.) | 1307 | TTTN | SSDNA/dsDNA | 24 |
| | | | (staggered) | |
| Cas12f (A. sulfuroxidans) | 422 | NTTR | dsDNA (staggered) | variable |

See generally, Wang, J., et al. (2020). Journal of Cellular and Molecular Medicine, 24(6), 3256-3270; Liu, G., et al. (2022). Molecular Cell, where N = any nucleotide, R = any purine, Y = any pyrimidine, W = A or T, and V = A, C or G.

TABLE 5
Exemplary vector components and related nucleotide sequences

| Component | Sequence |
|---|---|
| AAV ITR | TTGGCCACTCCCTCTCTGCGCGCTCGCTCGCTCACTGAGGCCGCCCGGGCAAAGCCCG |
| (SEQ ID NO: 241) | GGCGTCGGGCGACCTTTGGTCGCCCGGCCTCAGTGAGCGAGCGAGCGCGCAGAGAGG |
| | GAGTGGCCAACTCCATCACTAGGGGTTCCT |
| SaCas9 guide RNA | GTTTTAGTACTCTGGAAACAGAATCTACTAAAACAAGGCAAAATGCCGTGTTTATCTC |
| scaffold | GTCAACTTGTTGGCGAGATTTTTT |
| (SEQ ID NO: 242) | |
| KKH-SaCas9 | GTTTTAGTACTCTGGAAACAGAATCTACTAAAACAAGGCAAAATGCCGTGTTTATCTCGT |
| guide RNA | CAACTTGTTGGCGAGATTTTTT |
| scaffold | |
| (SEQ ID NO: 243) | |
| SpCas9 guide | GTTTTAGAGCTAGAAATAGCAAGTTAAAATAAGGCTAGTCCGTTATCAACTTGAAAAA |
| RNA scaffold | GTGGCACCGAGTCGGTGCTTTTTT |
| (SEQ ID NO: 244) | |
| U6 promoter | GAGGGCCTATTTCCCATGATTCCTTCATATTTGCATATACGATACAAGGCTGTTAGA |
| (SEQ ID NO: 245) | GAGATAATTGGAATTAATTTGACTGTAAACACAAAGATATTAGTACAAAATACGTGAC |
| | GTAGAAAGTAATAATTTCTTGGGTAGTTTGCAGTTTTAAAATTATGTTTTAAAATGGAC |
| | TATCATATGCTTACCGTAACTTGAAAGTATTTCGATTTCTTGGCTTTATATATCTTGTG |
| | GAAAGGACGAAACACC |
| CBh promoter | CGTTACATAACTTACGGTAAATGGCCCGCCTGGCTGACCGCCCAACGACCCCCGCCCA |
| (SEQ ID NO: 246) | TTGACGTCAATAGTAACGCCAATAGGGACTTTCCATTGACGTCAATGGGTGGAGTATTT |
| | ACGGTAAACTGCCCACTTGGCAGTACATCAAGTGTATCATATGCCAAGTACGCCCCCTA |
| | TTGACGTCAATGACGGTAAATGGCCCGCCTGGCATTGTGCCCAGTACATGACCTTATG |
| | GGACTTTCCTACTTGGCAGTACATCTACGTATTAGTCATCGCTATTACCATGGTCGAGG |
| | TGAGCCCCACGTTCTGCTTCACTCTCCCCATCTCCCCCCCCTCCCCACCCCCAATTTTG |
| | TATTTATTTATTTTTTAATTATTTTGTGCAGCGATGGGGGGGGGGGGGGGGGGGGGC |
| | GGGGCGAGGGGGGGGGGGGGGCGAGGCGGAGAGGTGCGGCGGCAGCCAATCAGAG |
| | CGGCGCGCTCCGAAAGTTTCCTTTTATGGCGAGGGGGGGGCGGCGGCGGCCCTATAA |
| | AAAGCGAAGCGCGCGGCGGGCGGGAGTCGCTGCGCGCTGCCTTCGCCCCGTGCCCC |
| | GCTCCGCCGCCGCCTCGCGCCGCCCGCCCCGGCTCTGACTGACCGCGTTACTCCCAC |
| | AGGTGAGCGGGCGGGACGGCCCTTCTCCTCCGGGCTGTAATTAGCTGAGCAAGAGGT |
| | AAGGGTTTAAGGGATGGTTGGTTGGTGGGGTATTAATGTTTAATTACCTGGAGCACCTG |
| | CCTGAAATCACTTTTTTTCAG |
| EFS promoter | TCGAGTGGCTCCGGTGCCCGTCAGTGGGCAGAGCGCACATCGCCCACAGTCCCCGA |
| (SEQ ID NO: 247) | GAAGTTGGGGGGAGGGGTCGGCAATTGAACCGGTGCCTAGAGAAGGTGGCGCGGG |
| | GTAAACTGGGAAAGTGATGTCGTGTACTGGCTCCGCCTTTTTCCCGAGGGTGGGGGA |
| | GAACCGTATATAAGTGCAGTAGTCGCCGTGAACGTTCTTTTTCGCAACGGGTTTGCCG |
| | CCAGAACACAGGTGTCGTGACCGCGGCCATGGTCTAGAGGATCCGGTACTCGAGGAA |
| | CTGAAAAACCAGAAAGTTAACTGGTAAGTTTAGTCTTTTTGTCTTTTATTTCAGGTCCC |
| | GGATCCGGTGGTGGTGCAAATCAAAGAACTGCTCCTCAGTGGATGTTGCCTTTACTTC |
| | TAG |
| hSyn promoter | AGTGCAAGTGGGTTTTAGGACCAGGATGAGGGGGGTGGGGGTGCCTACCTGACGAC |
| (SEQ ID NO: 248) | CGACCCCGACCCACTGGACAAGCACCCAACCCCCATTCCCCAAATTGCGCATCCCCTA |
| | TCAGAGAGGGGGAGGGGAAACAGGATGCGGCGAGGCGCGTGCGCACTGCCAGCTTC |
| | AGCACCGCGGACAGTGCCTTCGCCCCCGCCTGGCGGCGCGCGCCACCGCCGCCTCA |
| | GCACTGAAGGCGCGCTGACGTCACTCGCCGGTCCCCCGCAAACTCCCCTTCCCGGCC |
| | ACCTTGGTCGCGTCCGCGCCGCCGCCGGCCCAGCCGGACCGCACCACGCGAGGCGC |
| | GAGATAGGGGGGCACGGGCGCGACCATCTGCGCTGCGGCGCCGGCGACTCAGCGCT |
| | GCCTCAGTCTGCGGTGGGCAGCGGAGGAGTCGTGTCGTGCCTGAGAGCGCAG |
| hMeCP2 promoter | AGCTGAATGGGGTCCGCCTCTTTTCCCTGCCTAAACAGACAGGAACTCCTGCCA |
| (SEQ ID NO: 249) | ATTGAGGGCGTCACCGCTAAGGCTCCGCCCCAGCCTGGGCTCCACAACCAATG |
| | AAGGGTAATCTCGACAAAGAGCAAGGGGTGGGGCGCGGGCGCGCAGGTGCAG |
| | CAGCACACAGGCTGGTCGGGAGGGGGGGGCGCGACGTCTGCCGTGCGGGGT |
| | CCCGGCATCGGTTGCGCGC |
| JeT promoter | GAATTCGGGCGGAGTTAGGGCGGAGCCAATCAGCGTGCGCCGTTCCGAAAGTT |
| (SEQ ID NO: 250) | GCCTTTTATGGCTGGGCGGAGAATGGGGGGTGAACGCCGATGATTATATAAGG |
| | ACGCGCCGGGTGTGGCACAGCTAGTTCCGTCGCAGCCGGGATTTGGGTCGCG |
| | GTTCTTGTTTGTGGATCCCTGTGATA |
| SaCas9 | ATGGCCCCAAAGAAGAAGCGGAAGGTCGGTATCCACGGAGTCCCAGCAGCCAAGCGG |
| (SEQ ID NO: 251) | AACTACATCCTGGGCCTGGACATCGGCATCACCAGCGTGGGCTACGGCATCATCGACT |
| | ACGAGACACGGGACGTGATCGATGCCGGCGTGCGGCTGTTCAAAGAGGCCAACGTGG |
| | AAAACAACGAGGGCAGGCGGAGCAAGAGAGGCGCCAGAAGGCTGAAGCGGCGGAGG |
| | CGGCATAGAATCCAGAGAGTGAAGAAGCTGCTGTTCGACTACAACCTGCTGACCGACC |
| | ACAGCGAGCTGAGCGGCATCAACCCCTACGAGGCCAGAGTGAAGGGCCTGAGCCAGA |
| | AGCTGAGCGAGGAAGAGTTCTCTGCCGCCCTGCTGCACCTGGCCAAGAGAAGAGGCG |
| | TGCACAACGTGAACGAGGTGGAAGAGGACACCGGCAACGAGCTGTCCACCAAAGAGC |
| | AGATCAGCCGGAACAGCAAGGCCCTGGAAGAGAAATACGTGGCCGAACTGCAGCTGG |
| | AACGGCTGAAGAAAGACGGCGAAGTGCGGGGCAGCATCAACAGATTCAAGACCAGCG |
| | ACTACGTGAAAGAAGCCAAACAGCTGCTGAAGGTGCAGAAGGCCTACCACCAGCTGGA |
| | CCAGAGCTTCATCGACACCTACATCGACCTGCTGGAAACCCGGCGGACCTACTATGAG |
| | GGACCTGGCGAGGGCAGCCCCTTCGGCTGGAAGGACATCAAAGAATGGTACGAGATG |
| | CTGATGGGCCACTGCACCTACTTCCCCGAGGAACTGCGGAGCGTGAAGTACGCCTAC |
| | AACGCCGACCTGTACAACGCCCTGAACGACCTGAACAATCTCGTGATCACCAGGGACG |
| | AGAACGAGAAGCTGGAATATTACGAGAAGTTCCAGATCATCGAGAACGTGTTCAAGCA |
| | GAAGAAGAAGCCCACCCTGAAGCAGATCGCCAAAGAAATCCTCGTGAACGAAGAGGAT |
| | ATTAAGGGCTACAGAGTGACCAGCACCGGCAAGCCCGAGTTCACCAACCTGAAGGTGT |
| | ACCACGACATCAAGGACATTACCGCCCGGAAAGAGATTATTGAGAACGCCGAGCTGCT |
| | GGATCAGATTGCCAAGATCCTGACCATCTACCAGAGCAGCGAGGACATCCAGGAAGAA |
| | CTGACCAATCTGAACTCCGAGCTGACCCAGGAAGAGATCGAGCAGATCTCTAATCTGA |
| | AGGGCTATACCGGCACCCACAACCTGAGCCTGAAGGCCATCAACCTGATCCTGGACGA |
| | GCTGTGGCACACCAACGACAACCAGATCGCTATCTTCAACCGGCTGAAGCTGGTGCCC |
| | AAGAAGGTGGACCTGTCCCAGCAGAAAGAGATCCCCACCACCCTGGTGGACGACTTCA |
| | TCCTGAGCCCCGTCGTGAAGAGAAGCTTCATCCAGAGCATCAAAGTGATCAACGCCAT |
| | CATCAAGAAGTACGGCCTGCCCAACGACATCATTATCGAGCTGGCCCGCGAGAAGAAC |
| | TCCAAGGACGCCCAGAAAATGATCAACGAGATGCAGAAGCGGAACCGGCAGACCAAC |
| | GAGCGGATCGAGGAAATCATCCGGACCACCGGCAAAGAGAACGCCAAGTACCTGATC |
| | GAGAAGATCAAGCTGCACGACATGCAGGAAGGCAAGTGCCTGTACAGCCTGGAAGCC |
| | ATCCCTCTGGAAGATCTGCTGAACAACCCCTTCAACTATGAGGTGGACCACATCATCCC |
| | CAGAAGCGTGTCCTTCGACAACAGCTTCAACAACAAGGTGCTCGTGAAGCAGGAAGAA |
| | AACAGCAAGAAGGGCAACCGGACCCCATTCCAGTACCTGAGCAGCAGCGACAGCAAG |
| | ATCAGCTACGAAACCTTCAAGAAGCACATCCTGAATCTGGCCAAGGGCAAGGGCAGAA |
| | TCAGCAAGACCAAGAAAGAGTATCTGCTGGAAGAACGGGACATCAACAGGTTCTCCGT |
| | GCAGAAAGACTTCATCAACCGGAACCTGGTGGATACCAGATACGCCACCAGAGGCCTG |
| | ATGAACCTGCTGCGGAGCTACTTCAGAGTGAACAACCTGGACGTGAAAGTGAAGTCCA |
| | TCAATGGCGGCTTCACCAGCTTTCTGCGGCGGAAGTGGAAGTTTAAGAAAGAGCGGAA |
| | CAAGGGGTACAAGCACCACGCCGAGGACGCCCTGATCATTGCCAACGCCGATTTCATC |
| | TTCAAAGAGTGGAAGAAACTGGACAAGGCCAAAAAAGTGATGGAAAACCAGATGTTCG |
| | AGGAAAAGCAGGCCGAGAGCATGCCCGAGATCGAAACCGAGCAGGAGTACAAAGAGA |
| | TCTTCATCACCCCCCACCAGATCAAGCACATTAAGGACTTCAAGGACTACAAGTACAGC |
| | CACCGGGTGGACAAGAAGCCTAATAGAGAGCTGATTAACGACACCCTGTACTCCACCC |
| | GGAAGGACGACAAGGGCAACACCCTGATCGTGAACAATCTGAACGGCCTGTACGACAA |
| | GGACAATGACAAGCTGAAAAAGCTGATCAACAAGAGCCCCGAAAAGCTGCTGATGTAC |
| | CACCACGACCCCCAGACCTACCAGAAACTGAAGCTGATTATGGAACAGTACGGCGACG |
| | AGAAGAATCCCCTGTACAAGTACTACGAGGAAACCGGGAACTACCTGACCAAGTACTC |
| | CAAAAAGGACAACGGCCCCGTGATCAAGAAGATTAAGTATTACGGCAACAAACTGAAC |
| | GCCCATCTGGACATCACCGACGACTACCCCAACAGCAGAAACAAGGTCGTGAAGCTGT |
| | CCCTGAAGCCCTACAGATTCGACGTGTACCTGGACAATGGCGTGTACAAGTTCGTGAC |
| | CGTGAAGAATCTGGATGTGATCAAAAAAGAAAACTACTACGAAGTGAATAGCAAGTGCT |
| | ATGAGGAAGCTAAGAAGCTGAAGAAGATCAGCAACCAGGCCGAGTTTATCGCCTCCTT |
| | CTACAACAACGATCTGATCAAGATCAACGGCGAGCTGTATAGAGTGATCGGCGTGAAC |
| | AACGACCTGCTGAACCGGATCGAAGTGAACATGATCGACATCACCTACCGCGAGTACC |
| | TGGAAAACATGAACGACAAGAGGCCCCCCAGGATCATTAAGACAATCGCCTCCAAGAC |
| | CCAGAGCATTAAGAAGTACAGCACAGACATTCTGGGCAACCTGTATGAAGTGAAATCTA |
| | AGAAGCACCCTCAGATCATCAAAAAGGGCAAAAGGCCGGCGGCCACGAAAAAGGCCG |
| | GCCAGGCAAAAAAGAAAAAG |
| SaCas9-KKH | ATGGCCCCAAAGAAGAAGCGGAAGGTCGGTATCCACGGAGTCCCAGCAGCCAAGCGG |
| (SEQ ID NO: 252) | AACTACATCCTGGGCCTGGACATCGGCATCACCAGCGTGGGCTACGGCATCATCGACT |
| | ACGAGACACGGGACGTGATCGATGCCGGCGTGCGGCTGTTCAAAGAGGCCAACGTGG |
| | AAAACAACGAGGGCAGGCGGAGCAAGAGAGGCGCCAGAAGGCTGAAGCGGCGGAGG |
| | CGGCATAGAATCCAGAGAGTGAAGAAGCTGCTGTTCGACTACAACCTGCTGACCGACC |
| | ACAGCGAGCTGAGCGGCATCAACCCCTACGAGGCCAGAGTGAAGGGCCTGAGCCAGA |
| | AGCTGAGCGAGGAAGAGTTCTCTGCCGCCCTGCTGCACCTGGCCAAGAGAAGAGGCG |
| | TGCACAACGTGAACGAGGTGGAAGAGGACACCGGCAACGAGCTGTCCACCAAAGAGC |
| | AGATCAGCCGGAACAGCAAGGCCCTGGAAGAGAAATACGTGGCCGAACTGCAGCTGG |
| | AACGGCTGAAGAAAGACGGCGAAGTGCGGGGCAGCATCAACAGATTCAAGACCAGCG |
| | ACTACGTGAAAGAAGCCAAACAGCTGCTGAAGGTGCAGAAGGCCTACCACCAGCTGGA |
| | CCAGAGCTTCATCGACACCTACATCGACCTGCTGGAAACCCGGCGGACCTACTATGAG |
| | GGACCTGGCGAGGGCAGCCCCTTCGGCTGGAAGGACATCAAAGAATGGTACGAGATG |
| | CTGATGGGCCACTGCACCTACTTCCCCGAGGAACTGCGGAGCGTGAAGTACGCCTAC |
| | AACGCCGACCTGTACAACGCCCTGAACGACCTGAACAATCTCGTGATCACCAGGGACG |
| | AGAACGAGAAGCTGGAATATTACGAGAAGTTCCAGATCATCGAGAACGTGTTCAAGCA |
| | GAAGAAGAAGCCCACCCTGAAGCAGATCGCCAAAGAAATCCTCGTGAACGAAGAGGAT |
| | ATTAAGGGCTACAGAGTGACCAGCACCGGCAAGCCCGAGTTCACCAACCTGAAGGTGT |
| | ACCACGACATCAAGGACATTACCGCCCGGAAAGAGATTATTGAGAACGCCGAGCTGCT |
| | GGATCAGATTGCCAAGATCCTGACCATCTACCAGAGCAGCGAGGACATCCAGGAAGAA |
| | CTGACCAATCTGAACTCCGAGCTGACCCAGGAAGAGATCGAGCAGATCTCTAATCTGA |
| | AGGGCTATACCGGCACCCACAACCTGAGCCTGAAGGCCATCAACCTGATCCTGGACGA |
| | GCTGTGGCACACCAACGACAACCAGATCGCTATCTTCAACCGGCTGAAGCTGGTGCCC |
| | AAGAAGGTGGACCTGTCCCAGCAGAAAGAGATCCCCACCACCCTGGTGGACGACTTCA |
| | TCCTGAGCCCCGTCGTGAAGAGAAGCTTCATCCAGAGCATCAAAGTGATCAACGCCAT |
| | CATCAAGAAGTACGGCCTGCCCAACGACATCATTATCGAGCTGGCCCGCGAGAAGAAC |
| | TCCAAGGACGCCCAGAAAATGATCAACGAGATGCAGAAGCGGAACCGGCAGACCAAC |
| | GAGCGGATCGAGGAAATCATCCGGACCACCGGCAAAGAGAACGCCAAGTACCTGATC |
| | GAGAAGATCAAGCTGCACGACATGCAGGAAGGCAAGTGCCTGTACAGCCTGGAAGCC |
| | ATCCCTCTGGAAGATCTGCTGAACAACCCCTTCAACTATGAGGTGGACCACATCATCCC |
| | CAGAAGCGTGTCCTTCGACAACAGCTTCAACAACAAGGTGCTCGTGAAGCAGGAAGAA |
| | AACAGCAAGAAGGGCAACCGGACCCCATTCCAGTACCTGAGCAGCAGCGACAGCAAG |
| | ATCAGCTACGAAACCTTCAAGAAGCACATCCTGAATCTGGCCAAGGGCAAGGGCAGAA |
| | TCAGCAAGACCAAGAAAGAGTATCTGCTGGAAGAACGGGACATCAACAGGTTCTCCGT |
| | GCAGAAAGACTTCATCAACCGGAACCTGGTGGATACCAGATACGCCACCAGAGGCCTG |
| | ATGAACCTGCTGCGGAGCTACTTCAGAGTGAACAACCTGGACGTGAAAGTGAAGTCCA |
| | TCAATGGCGGCTTCACCAGCTTTCTGCGGCGGAAGTGGAAGTTTAAGAAAGAGCGGAA |
| | CAAGGGGTACAAGCACCACGCCGAGGACGCCCTGATCATTGCCAACGCCGATTTCATC |
| | TTCAAAGAGTGGAAGAAACTGGACAAGGCCAAAAAAGTGATGGAAAACCAGATGTTCG |
| | AGGAAAAGCAGGCCGAGAGCATGCCCGAGATCGAAACCGAGCAGGAGTACAAAGAGA |
| | TCTTCATCACCCCCCACCAGATCAAGCACATTAAGGACTTCAAGGACTACAAGTACAGC |
| | CACCGGGTGGACAAGAAGCCTAATAGAAAGCTGATTAACGACACCCTGTACTCCACCC |
| | GGAAGGACGACAAGGGCAACACCCTGATCGTGAACAATCTGAACGGCCTGTACGACAA |
| | GGACAATGACAAGCTGAAAAAGCTGATCAACAAGAGCCCCGAAAAGCTGCTGATGTAC |
| | CACCACGACCCCCAGACCTACCAGAAACTGAAGCTGATTATGGAACAGTACGGCGACG |
| | AGAAGAATCCCCTGTACAAGTACTACGAGGAAACCGGGAACTACCTGACCAAGTACTC |
| | CAAAAAGGACAACGGCCCCGTGATCAAGAAGATTAAGTATTACGGCAACAAACTGAAC |
| | GCCCATCTGGACATCACCGACGACTACCCCAACAGCAGAAACAAGGTCGTGAAGCTGT |
| | CCCTGAAGCCCTACAGATTCGACGTGTACCTGGACAATGGCGTGTACAAGTTCGTGAC |
| | CGTGAAGAATCTGGATGTGATCAAAAAAGAAAACTACTACGAAGTGAATAGCAAGTGCT |
| | ATGAGGAAGCTAAGAAGCTGAAGAAGATCAGCAACCAGGCCGAGTTTATCGCCTCCTT |
| | CTACAAGAACGATCTGATCAAGATCAACGGCGAGCTGTATAGAGTGATCGGCGTGAAC |
| | AACGACCTGCTGAACCGGATCGAAGTGAACATGATCGACATCACCTACCGCGAGTACC |
| | TGGAAAACATGAACGACAAGAGGCCCCCCCACATCATTAAGACAATCGCCTCCAAGAC |
| | CCAGAGCATTAAGAAGTACAGCACAGACATTCTGGGCAACCTGTATGAAGTGAAATCTA |
| | AGAAGCACCCTCAGATCATCAAAAAGGGCAAAAGGCCGGCGGCCACGAAAAAGGCCG |
| | GCCAGGCAAAAAAGAAAAAG |
| SpCas9 | ATGCCAAAGAAGAAGCGGAAGGTCGGTATCCACGGAGTCCCAGCAGCCGACAAGAAGT |
| (SEQ ID NO: 253) | ACAGCATCGGCCTGGACATCGGCACCAACTCTGTGGGCTGGGCCGTGATCACCGACGA |
| | GTACAAGGTGCCCAGCAAGAAATTCAAGGTGCTGGGCAACACCGACCGGCACAGCATC |
| | AAGAAGAACCTGATCGGAGCCCTGCTGTTCGACAGCGGCGAAACAGCCGAGGCCACCC |
| | GGCTGAAGAGAACCGCCAGAAGAAGATACACCAGACGGAAGAACCGGATCTGCTATCT |
| | GCAAGAGATCTTCAGCAACGAGATGGCCAAGGTGGACGACAGCTTCTTCCACAGACTGG |
| | AAGAGTCCTTCCTGGTGGAAGAGGATAAGAAGCACGAGCGGCACCCCATCTTCGGCAA |
| | CATCGTGGACGAGGTGGCCTACCACGAGAAGTACCCCACCATCTACCACCTGAGAAAGA |
| | AACTGGTGGACAGCACCGACAAGGCCGACCTGCGGCTGATCTATCTGGCCCTGGCCCA |
| | CATGATCAAGTTCCGGGGCCACTTCCTGATCGAGGGCGACCTGAACCCCGACAACAGC |
| | GACGTGGACAAGCTGTTCATCCAGCTGGTGCAGACCTACAACCAGCTGTTCGAGGAAAA |
| | CCCCATCAACGCCAGCGGCGTGGACGCCAAGGCCATCCTGTCTGCCAGACTGAGCAAG |
| | AGCAGACGGCTGGAAAATCTGATCGCCCAGCTGCCCGGCGAGAAGAAGAATGGCCTGT |
| | TCGGAAACCTGATTGCCCTGAGCCTGGGCCTGACCCCCAACTTCAAGAGCAACTTCGAC |
| | CTGGCCGAGGATGCCAAACTGCAGCTGAGCAAGGACACCTACGACGACGACCTGGACA |
| | ACCTGCTGGCCCAGATCGGCGACCAGTACGCCGACCTGTTTCTGGCCGCCAAGAACCT |
| | GTCCGACGCCATCCTGCTGAGCGACATCCTGAGAGTGAACACCGAGATCACCAAGGCC |
| | CCCCTGAGCGCCTCTATGATCAAGAGATACGACGAGCACCACCAGGACCTGACCCTGCT |
| | GAAAGCTCTCGTGCGGCAGCAGCTGCCTGAGAAGTACAAAGAGATTTTCTTCGACCAGA |
| | GCAAGAACGGCTACGCCGGCTACATTGACGGCGGAGCCAGCCAGGAAGAGTTCTACAA |
| | GTTCATCAAGCCCATCCTGGAAAAGATGGACGGCACCGAGGAACTGCTCGTGAAGCTGA |
| | ACAGAGAGGACCTGCTGCGGAAGCAGCGGACCTTCGACAACGGCAGCATCCCCCACCA |
| | GATCCACCTGGGAGAGCTGCACGCCATTCTGCGGCGGCAGGAAGATTTTTACCCATTCC |
| | TGAAGGACAACCGGGAAAAGATCGAGAAGATCCTGACCTTCCGCATCCCCTACTACGTG |
| | GGCCCTCTGGCCAGGGGAAACAGCAGATTCGCCTGGATGACCAGAAAGAGCGAGGAAA |
| | CCATCACCCCCTGGAACTTCGAGGAAGTGGTGGACAAGGGCGCTTCCGCCCAGAGCTT |
| | CATCGAGCGGATGACCAACTTCGATAAGAACCTGCCCAACGAGAAGGTGCTGCCCAAG |
| | CACAGCCTGCTGTACGAGTACTTCACCGTGTATAACGAGCTGACCAAAGTGAAATACGT |
| | GACCGAGGGAATGAGAAAGCCCGCCTTCCTGAGCGGCGAGCAGAAAAAGGCCATCGTG |
| | GACCTGCTGTTCAAGACCAACCGGAAAGTGACCGTGAAGCAGCTGAAAGAGGACTACTT |
| | CAAGAAAATCGAGTGCTTCGACTCCGTGGAAATCTCCGGCGTGGAAGATCGGTTCAACG |
| | CCTCCCTGGGCACATACCACGATCTGCTGAAAATTATCAAGGACAAGGACTTCCTGGAC |
| | AATGAGGAAAACGAGGACATTCTGGAAGATATCGTGCTGACCCTGACACTGTTTGAGGA |
| | CAGAGAGATGATCGAGGAACGGCTGAAAACCTATGCCCACCTGTTCGACGACAAAGTGA |
| | TGAAGCAGCTGAAGCGGCGGAGATACACCGGCTGGGGCAGGCTGAGCCGGAAGCTGA |
| | TCAACGGCATCCGGGACAAGCAGTCCGGCAAGACAATCCTGGATTTCCTGAAGTCCGAC |
| | GGCTTCGCCAACAGAAACTTCATGCAGCTGATCCACGACGACAGCCTGACCTTTAAAGA |
| | GGACATCCAGAAAGCCCAGGTGTCCGGCCAGGGCGATAGCCTGCACGAGCACATTGCC |
| | AATCTGGCCGGCAGCCCCGCCATTAAGAAGGGCATCCTGCAGACAGTGAAGGTGGTGG |
| | ACGAGCTCGTGAAAGTGATGGGCCGGCACAAGCCCGAGAACATCGTGATCGAAATGGC |
| | CAGAGAGAACCAGACCACCCAGAAGGGACAGAAGAACAGCCGCGAGAGAATGAAGCGG |
| | ATCGAAGAGGGCATCAAAGAGCTGGGCAGCCAGATCCTGAAAGAACACCCCGTGGAAA |
| | ACACCCAGCTGCAGAACGAGAAGCTGTACCTGTACTACCTGCAGAATGGGGGGATATG |
| | TACGTGGACCAGGAACTGGACATCAACCGGCTGTCCGACTACGATGTGGACCATATCGT |
| | GCCTCAGAGCTTTCTGAAGGACGACTCCATCGACAACAAGGTGCTGACCAGAAGCGACA |
| | AGAACCGGGGCAAGAGCGACAACGTGCCCTCCGAAGAGGTCGTGAAGAAGATGAAGAA |
| | CTACTGGCGGCAGCTGCTGAACGCCAAGCTGATTACCCAGAGAAAGTTCGACAATCTGA |
| | CCAAGGCCGAGAGAGGCGGCCTGAGCGAACTGGATAAGGCCGGCTTCATCAAGAGACA |
| | GCTGGTGGAAACCCGGCAGATCACAAAGCACGTGGCACAGATCCTGGACTCCCGGATG |
| | AACACTAAGTACGACGAGAATGACAAGCTGATCCGGGAAGTGAAAGTGATCACCCTGAA |
| | GTCCAAGCTGGTGTCCGATTTCCGGAAGGATTTCCAGTTTTACAAAGTGCGCGAGATCA |
| | ACAACTACCACCACGCCCACGACGCCTACCTGAACGCCGTCGTGGGAACCGCCCTGAT |
| | CAAAAAGTACCCTAAGCTGGAAAGCGAGTTCGTGTACGGCGACTACAAGGTGTACGACG |
| | TGCGGAAGATGATCGCCAAGAGCGAGCAGGAAATCGGCAAGGCTACCGCCAAGTACTT |
| | CTTCTACAGCAACATCATGAACTTTTTCAAGACCGAGATTACCCTGGCCAACGGCGAGAT |
| | CCGGAAGCGGCCTCTGATCGAGACAAACGGCGAAACCGGGGAGATCGTGTGGGATAAG |
| | GGCCGGGATTTTGCCACCGTGCGGAAAGTGCTGAGCATGCCCCAAGTGAATATCGTGA |
| | AAAAGACCGAGGTGCAGACAGGCGGCTTCAGCAAAGAGTCTATCCTGCCCAAGAGGAA |
| | CAGCGATAAGCTGATCGCCAGAAAGAAGGACTGGGACCCTAAGAAGTACGGCGGCTTC |
| | GACAGCCCCACCGTGGCCTATTCTGTGCTGGTGGTGGCCAAAGTGGAAAAGGGCAAGT |
| | CCAAGAAACTGAAGAGTGTGAAAGAGCTGCTGGGGATCACCATCATGGAAAGAAGCAGC |
| | TTCGAGAAGAATCCCATCGACTTTCTGGAAGCCAAGGGCTACAAAGAAGTGAAAAAGGA |
| | CCTGATCATCAAGCTGCCTAAGTACTCCCTGTTCGAGCTGGAAAACGGCCGGAAGAGAA |
| | TGCTGGCCTCTGCCGGCGAACTGCAGAAGGGAAACGAACTGGCCCTGCCCTCCAAATA |
| | TGTGAACTTCCTGTACCTGGCCAGCCACTATGAGAAGCTGAAGGGCTCCCCCGAGGATA |
| | ATGAGCAGAAACAGCTGTTTGTGGAACAGCACAAGCACTACCTGGACGAGATCATCGAG |
| | CAGATCAGCGAGTTCTCCAAGAGAGTGATCCTGGCCGACGCTAATCTGGACAAAGTGCT |
| | GTCCGCCTACAACAAGCACCGGGATAAGCCCATCAGAGAGCAGGCCGAGAATATCATC |
| | CACCTGTTTACCCTGACCAATCTGGGAGCCCCTGCCGCCTTCAAGTACTTTGACACCAC |
| | CATCGACCGGAAGAGGTACACCAGCACCAAAGAGGTGCTGGACGCCACCCTGATCCAC |
| | CAGAGCATCACCGGCCTGTACGAGACACGGATCGACCTGTCTCAGCTGGGAGGCGACA |
| | AAAGGCCGGCGGCCACGAAAAAGGCCGGCCAGGCAAAAAAGAAAAAG |
| Mini polyadenylation | TAGCAATAAAGGATCGTTTATTTTCATTGGAAGCGTGTGTTGGTTTTTTGATCAGGCGCG |
| signal | |
| (SEQ ID NO: 254) | |
| bGH polyadenylation | CTAGAGCTCGCTGATCAGCCTCGACTGTGCCTTCTAGTTGCCAGCCATCTGTTGTTTGC |
| signal | CCCTCCCCCGTGCCTTCCTTGACCCTGGAAGGTGCCACTCCCACTGTCCTTTCCTAATAA |
| (SEQ ID NO: 255) | AATGAGGAAATTGCATCGCATTGTCTGAGTAGGTGTCATTCTATTCTGGGGGGTGGGGT |
| | GGGGCAGGACAGCAAGGGGGAGGATTGGGAAGAGAATAGCAGGCATGCTGGGGA |

TABLE 6
Experimental design for in vivo gene editing

| Mouse | Experimental | No. animals | Terminal time |
|---|---|---|---|
| strain | Condition | (Male/Female) | point (mpi) |
| Hu97/18 | 0.9% saline (control) | 4 (2/2) | 2 |
| | | 6 (3/3) | 15 |
| Hu97/18 | AAV5-SaCas9/SEQ ID | 4 (2/2) | 2 |
| | NO: 1 | 6 (3/3) | 15 |
| Hu97/18 | AAV5-SaCas9/SEQ ID | 4 (2/2) | 2 |
| | NO: 2 | 6 (3/3) | 15 |
| Hu97/18 | AAV5-iCre | 4 (2/2) | 2 |

1. A CRISPR based gene editing system comprising one or more polynucleotides, wherein the one or more polynucleotides encode a composition that comprises:
(a) a Cas protein or a fusion protein comprising the Cas protein or its component, and
(b) a gRNA, wherein the gRNA comprises a sequence selected from SEQ ID NOs: 1-120.

2. The gene editing system of claim 1, wherein the gRNA targets an exon of a HTT gene selected from any one of exons 1-63, exon 65, and exon 66.

3. The gene editing system of claim 1, wherein the Cas protein is a type II Cas enzyme or a type V Cas enzyme.

4. The gene editing system of claim 3, wherein the Cas protein is a Cas9 protein.

5. The gene editing system of claim 4, wherein the Cas9 protein is a SaCas9 protein; and wherein the gRNA comprises a sequence selected from any one of SEQ ID NOs: 1-40.

6. The gene editing system of claim 5, wherein the gRNA comprises SEQ ID NO: 1 or SEQ ID NO: 2.

7. The gene editing system of claim 6, wherein the SaCa9 protein recognizes a protospacer-adjacent motif (PAM) comprising SEQ ID NO: 121 or 122.

8.-11. (canceled)

12. The gene editing system of claim 4, wherein the Cas9 protein is a KKH-SaCas9 protein; and wherein the gRNA comprises a sequence selected from any one of SEQ ID NOs: 41-80.

13. (canceled)

14. The gene editing system of claim 12, wherein the KKH-SaCa9 protein recognizes a PAM comprising a sequence of SEQ ID NO: 161 or 162.

15.-17. (canceled)

18. The gene editing system of claim 4, wherein the Cas9 protein is a SpCas9 protein; and wherein the gRNA comprises a sequence selected from any one of SEQ ID NOs: 81-120.

19. (canceled)

20. The gene editing system of claim 18, wherein the SpCas9 protein recognizes a PAM comprising a sequence of SEQ ID NO: 201-212.

21.-54. (canceled)

55. The gene editing system of claim 1, wherein the system introduces a double stranded break at a target nucleic acid sequence.

56. The gene editing system of claim 4, wherein the expression of the Cas9 protein is driven by a constitutive promoter or a neuron-specific promoter, wherein the consitutive promoter is a CBh promoter, a EFS promoter, an SCP1 promoter, an SCP3 promoter, or a JeT promoter, and the neuron-specific promoter is a E/hSyn promoter or a E/hMeCP2 promoter.

57.-58. (canceled)

59. The gene editing system of claim 1, wherein the Cas protein and the gRNA are encoded by a single vector.

60. The gene editing system of claim 1, wherein the Cas protein is encoded by a first vector and the gRNA is encoded by a second vector.

61. A viral vector expressing the gene editing system of claim 1.

62. (canceled)

63. The viral vector of claim 61, wherein the viral vector is an adeno-associated virus (AAV) vector, selected from AAV1, AAV2, AAV3, AAV4, AAV5, AAV6, AAV7, AAV8, AAV9, AAV-10, AAV-11, AAV-12, AAV-13, AAVrh.74, or a recombinant variant thereof.

64.-66. (canceled)

67. A cell comprising
the gene editing system of claim 1.

68.-75. (canceled)

76. A method of treating Huntington's Disease in a patient in need thereof, the method comprising administering to the patient the gene editing system of claim 1, wherein the gene editing system is administered to the patient intravenously, intracranially, or a combination thereof.

77.-78. (canceled)

79. The method of claim 76, wherein the detectable amount of huntingtin protein is reduced by at least about 50%, at least about 55% at least about 60%, at least about 70%, or at least about 75% as compared to an unmodified control.

80.-83. (canceled)
